SEQUENCE LISTING (58 sequences)

SEQ ID NO: 1 - DNA, 1893 bp, Cupressus gigantea
atggcagcacttagtttttctaccatctccaccactgccaactatgcaattcctaagctgccatcttcaaatcttcaagggcggtccagagatacaaaataccatgtcttgataagatctgcaggggcctctaatttcaacttcaatggaagaaagaagttcaatccaagaacacatcagaaagtcaaatctttgagtgttgaagattcctccaatgtacttattaaggctgagaaagagaacagagagaaaaaatcatttggttcgatgtgcatagatgatgatattacaaagataaaaatgttgttctcggaaattaaaagaagatattcatctctttctgcttatgatacagtgcacggtgcttatcctatcaatttgattcttggcctctcaatggtaaaccacttggagagcttgggtatagcaagacatttcaaacttgagataaaagaggttctagatgatgtctatagattttggatagagaatgaaatggttgaaggaatgccatcagcatcagacataatgaccagttctatcagtttcaggatcttgcggtggaatggatatgacgtgtctccagatgtctttttcagttacctgagggatagtgacttcctactatttttagaaaatagtgaccaagctactatagcattgctgaatttatacaaggcatctcagatgatgttcccaggcgaaagaattcttgaagaagcaaaattgttctcccaaaattatcttgaaaatatcaagatgggcaatcaaagtggagctctcaaagacattgaagcagaggttaagcatgcactggatgtcccttggatggctaatttggagagaatagaacatctgcgatacatgaaagcatttgcttttgacaatatttggattggagagacctcttctaggatcccatttacaggaaaggatttatttttatcttttgccaaacaagacttcaatatctgccaagcagctcaacaagaggatttgaaggcgttggaaaaatggagtgtagactctaaacttggtgacctacactttgctagacaaaagcttgtagcctgctatttctctgccgcctcaatactttttagccctgaaatgtctgctactcggattgtgtggactaaaaatgctgttctcattacactaatggatgacttttatgatgtggagggttccatygaagatattcaatcctttgttgaagcggttaagagatgggacccaatagttttatgtaattgttcagctaatgttaaaattttattctctgctatttataacactgtgaatgatattgctcaagatatatggattttccagggctgggacattagcactcagttaagagaaatttggtacgaactggcaatctcaatgatgaaggaggctgaatggaccaaaactggatttataccatccttggaagaatacatggaaaatgggagaacatcaattgccttggaaccaattatttttacatcactattttttattggccctaagctttccgagcaaatgatccaccatcacgagtataagaggcttttgcaactcatgaatacttgtgggcgccttttgaatgacattcagagcgacaagagggagatgaagcaaggaaagctcacctctgtatcattgttcgtgaaagaatacccaattgaggatgctactaagtggattagactcactgtaagtgaaaacacacaaaatatactcaaaaaaatattgcaaccaagtgagatccctcgtgaatgcaagcagatattttggaatatggtcaaaattatccaactattctgcctccacagtgatgaattcacatctcctaccacaatgcttgaacatatgaaggcagttcttttttatcctgttttataa

SEQ ID NO: 2 - DNA, 1896 bp, Cupressus gigantea
atggcagcacttagtttttctaccatctccaccactgccaactatgcaattcctaagctgccatcttcaaatcttcaagggcggtccagagatacaaaataccatgtcttgataagatctgcaggggcctctaatttcaacttcaatggaagaaagaagttcaatccaagaacacatcagaaagtcaaatctttgagtgttgaagattcctccaatgtacttattaaggctgagaaagagaacagagagaaaaaatcatttggttcgatgtgcatagacgataatattacaaggataaatatgttgttcttggaaattaagagaagatattcatctctttctgcttatgatacagtgcacggtgcttatcctatcaatttgattcttggcctctcaatggtaaatcatttggagagcttgggtatagcaagacatttcaaacttgagataaaagaggttctagatgatgtctatagattttggatagagaatgaaatggttaaaggaatgccatcagcatcagacataatgaccagttctatcagtttcaagatcttgcggtggaatggatatgacgtgtctccagatgtctttttcagttacctgagggatagtgacttcctaccatttctagaaaatagtggccaagctactatggcattgctgaatttatacaaggcatctcagatgatgttcccaggggaaagaattcttgaagaagcaaaatcgttctcccaaaattatcttgaaaatatcaaggtgggcgatcaaagtggagctctcaaagatattgaagcagaggttaagcatgcactggatgttccttggatggctggtttggagagaatagaacatctgcgatacatgaaagcatttgcttttgatgatatttggattggagagacctcttctaggatcccatttacaggaaaggatttatttttatctttagccaaacaagacttcaatatctgccaagaagctcaacaagaggatttgaaagcgttggaaaaatggagtgtagactctaaacttggtgacctacactttgctagacaaaagcttgtagcctgctatttctctgccgcctcaacactttttagccctgaaatgtctactactcggattgtgtggactaaaaatgctgttctcactactctaatggatgacttttatgatgtggagggttccattgaagatattcaatcctttgttgaagcagttaagagatgggacccaatagttttatgtaattgttcagctaatgtgaaaaaattattctctgctatttataacactgtgaatgatattgctcaagatatatggattttccagggctgggacattagcactcagttaagagaaatttggtacgaactggcaatctcaatgatgaaggaggctgaatggaccaaaactggatttataccatcgctggaagaatacatggaaaatgggagaacgtcaattgccttggaaccaattatttttacatcactattttttcttggccctaagctttccgagcaaatgatccacgagtataagagacttatgcaactcctgaatacttgtgggcgccttttgaatgacagtcagagctacaagagggagataaagcaaggaaagctcaactctttatcattgttcgtgaaagaatatcccgcaacatctattgaggatgctactgagtggattagactcactgtaagtgaaagcagacaaaatatactcagaaatttattgcaaccaagtgagatccctcgtgaatgcaagcagatattttggaatatggtcaaaattatccaactattctacctccacagtgatgaattcacatctcctactgcaatgcttgaacatatgcaagcagttctttttaatcctgttttataa

SEQ ID NO: 3 - protein, 630 aa, Cupressus gigantea
  UNSURE 398
MAALSFSTISTTANYAIPKLPSSNLQGRSRDTKYHVLIRSAGASNFNFNGRKKFNPRTHQKVKSLSVEDSSNVLIKAEKENREKKSFGSMCIDDDITKIKMLFSEIKRRYSSLSAYDTVHGAYPINLILGLSMVNHLESLGIARHFKLEIKEVLDDVYRFWIENEMVEGMPSASDIMTSSISFRILRWNGYDVSPDVFFSYLRDSDFLLFLENSDQATIALLNLYKASQMMFPGERILEEAKLFSQNYLENIKMGNQSGALKDIEAEVKHALDVPWMANLERIEHLRYMKAFAFDNIWIGETSSRIPFTGKDLFLSFAKQDFNICQAAQQEDLKALEKWSVDSKLGDLHFARQKLVACYFSAASILFSPEMSATRIVWTKNAVLITLMDDFYDVEGSIEDIQSFVEAVKRWDPIVLCNCSANVKILFSAIYNTVNDIAQDIWIFQGWDISTQLREIWYELAISMMKEAEWTKTGFIPSLEEYMENGRTSIALEPIIFTSLFFIGPKLSEQMIHHHEYKRLLQLMNTCGRLLNDIQSDKREMKQGKLTSVSLFVKEYPIEDATKWIRLTVSENTQNILKKILQPSEIPRECKQIFWNMVKIIQLFCLHSDEFTSPTTMLEHMKAVLFYPVL

SEQ ID NO: 4 - protein, 631 aa, Cupressus gigantea
MAALSFSTISTTANYAIPKLPSSNLQGRSRDTKYHVLIRSAGASNFNFNGRKKFNPRTHQKVKSLSVEDSSNVLIKAEKENREKKSFGSMCIDDNITRINMLFLEIKRRYSSLSAYDTVHGAYPINLILGLSMVNHLESLGIARHFKLEIKEVLDDVYRFWIENEMVKGMPSASDIMTSSISFKILRWNGYDVSPDVFFSYLRDSDFLPFLENSGQATMALLNLYKASQMMFPGERILEEAKSFSQNYLENIKVGDQSGALKDIEAEVKHALDVPWMAGLERIEHLRYMKAFAFDDIWIGETSSRIPFTGKDLFLSLAKQDFNICQEAQQEDLKALEKWSVDSKLGDLHFARQKLVACYFSAASTLFSPEMSTTRIVWTKNAVLTTLMDDFYDVEGSIEDIQSFVEAVKRWDPIVLCNCSANVKKLFSAIYNTVNDIAQDIWIFQGWDISTQLREIWYELAISMMKEAEWTKTGFIPSLEEYMENGRTSIALEPIIFTSLFFLGPKLSEQMIHEYKRLMQLLNTCGRLLNDSQSYKREIKQGKLNSLSLFVKEYPATSIEDATEWIRLTVSESRQNILRNLLQPSEIPRECKQIFWNMVKIIQLFYLHSDEFTSPTAMLEHMQAVLFNPVL

SEQ ID NO: 5 - protein, 549 aa, Artificial Sequence
  truncated protein
  UNSURE 317
MEKKSFGSMCIDDDITKIKMLFSEIKRRYSSLSAYDTVHGAYPINLILGLSMVNHLESLGIARHFKLEIKEVLDDVYRFWIENEMVEGMPSASDIMTSSISFRILRWNGYDVSPDVFFSYLRDSDFLLFLENSDQATIALLNLYKASQMMFPGERILEEAKLFSQNYLENIKMGNQSGALKDIEAEVKHALDVPWMANLERIEHLRYMKAFAFDNIWIGETSSRIPFTGKDLFLSFAKQDFNICQAAQQEDLKALEKWSVDSKLGDLHFARQKLVACYFSAASILFSPEMSATRIVWTKNAVLITLMDDFYDVEGSIEDIQSFVEAVKRWDPIVLCNCSANVKILFSAIYNTVNDIAQDIWIFQGWDISTQLREIWYELAISMMKEAEWTKTGFIPSLEEYMENGRTSIALEPIIFTSLFFIGPKLSEQMIHHHEYKRLLQLMNTCGRLLNDIQSDKREMKQGKLTSVSLFVKEYPIEDATKWIRLTVSENTQNILKKILQPSEIPRECKQIFWNMVKIIQLFCLHSDEFTSPTTMLEHMKAVLFYPVL

SEQ ID NO: 6 - protein, 550 aa, Artificial Sequence
  truncated protein
MENKSFGSMCIDDNITRINMLFLEIKRRYSSLSAYDTVHGAYPINLILGLSMVNHLESLGIARHFKLEIKEVLDDVYRFWIENEMVKGMPSASDIMTSSISFKILRWNGYDVSPDVFFSYLRDSDFLPFLENSGQATMALLNLYKASQMMFPGERILEEAKSFSQNYLENIKVGDQSGALKDIEAEVKHALDVPWMAGLERIEHLRYMKAFAFDDIWIGETSSRIPFTGKDLFLSLAKQDFNICQEAQQEDLKALEKWSVDSKLGDLHFARQKLVACYFSAASTLFSPEMSTTRIVWTKNAVLTTLMDDFYDVEGSIEDIQSFVEAVKRWDPIVLCNCSANVKKLFSAIYNTVNDIAQDIWIFQGWDISTQLREIWYELAISMMKEAEWTKTGFIPSLEEYMENGRTSIALEPIIFTSLFFLGPKLSEQMIHEYKRLMQLLNTCGRLLNDSQSYKREIKQGKLTSLSLFVKEYPAHLIEDATEWIRLTVSESRQNILRNLLQPSEIPRECKQIFWNMVKIIQLFYLHSDEFTSPTAMLEHMQAVLFNPVL

SEQ ID NO: 7 - protein, 550 aa, Artificial Sequence
  truncated protein
MEKKSFGSMCIDDNITRINMLFLEIKRRYSSLSAYDTVHGAYPINLILGLSMVNHLESLGIARHFKLEIKEVLDDVYRFWIENEMVKGMPSASDIMTSSISFKILRWNGYDVSPDVFFSYLRDSDFLPFLENSGQATMALLNLYKASQMMFPGERILEEAKSFSQNYLENIKVGDQSGALKDIEAEVKHALDVPWMAGLERIEHLRYMKAFAFDDIWIGETSSRIPFTGKDLFLSLAKQDFNICQEAQQEDLKALEKWSVDSKLGDLHFARQKLVACYFSAASTLFSPEMSTTRIVWTKNAVLTTLMDDFYDVEGSIEDIQSFVEAVKRWDPIVLCNCSANVKKLFSAIYNTVNDIAQDIWIFQGWDISTQLREIWYELAISMMKEAEWTKTGFIPSLEEYMENGRTSIALEPIIFTSLFFLGPKLSEQMIHEYKRLMQLLNTCGRLLNDSQSYKREIKQGKLNSLSLFVKEYPATSIEDATEWIRLTVSESRQNILRNLLQPSEIPRECKQIFWNMVKIIQLFYLHSDEFTSPTAMLEHMQAVLFNPVL

SEQ ID NO: 8 - protein, 921 aa, Artificial Sequence
  fusion protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGGGAAMEKKSFGSMCIDDDITKIKMLFSEIKRRYSSLSAYDTVHGAYPINLILGLSMVNHLESLGIARHFKLEIKEVLDDVYRFWIENEMVEGMPSASDIMTSSISFRILRWNGYDVSPDVFFSYLRDSDFLLFLENSDQATIALLNLYKASQMMFPGERILEEAKLFSQNYLENIKMGNQSGALKDIEAEVKHALDVPWMANLERIEHLRYMKAFAFDNIWIGETSSRIPFTGKDLFLSFAKQDFNICQAAQQEDLKALEKWSVDSKLGDLHFARQKLVACYFSAASILFSPEMSATRIVWTKNAVLITLMDDFYDVEGSIEDIQSFVEAVKRWDPIVLCNCSANVKILFSAIYNTVNDIAQDIWIFQGWDISTQLREIWYELAISMMKEAEWTKTGFIPSLEEYMENGRTSIALEPIIFTSLFFIGPKLSEQMIHHHEYKRLLQLMNTCGRLLNDIQSDKREMKQGKLTSVSLFVKEYPIEDATKWIRLTVSENTQNILKKILQPSEIPRECKQIFWNMVKIIQLFCLHSDEFTSPTTMLEHMKAVLFYPVL

SEQ ID NO: 9 - protein, 922 aa, Artificial Sequence
  fusion protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGGGAAMENKSFGSMCIDDNITRINMLFLEIKRRYSSLSAYDTVHGAYPINLILGLSMVNHLESLGIARHFKLEIKEVLDDVYRFWIENEMVKGMPSASDIMTSSISFKILRWNGYDVSPDVFFSYLRDSDFLPFLENSGQATMALLNLYKASQMMFPGERILEEAKSFSQNYLENIKVGDQSGALKDIEAEVKHALDVPWMAGLERIEHLRYMKAFAFDDIWIGETSSRIPFTGKDLFLSLAKQDFNICQEAQQEDLKALEKWSVDSKLGDLHFARQKLVACYFSAASTLFSPEMSTTRIVWTKNAVLTTLMDDFYDVEGSIEDIQSFVEAVKRWDPIVLCNCSANVKKLFSAIYNTVNDIAQDIWIFQGWDISTQLREIWYELAISMMKEAEWTKTGFIPSLEEYMENGRTSIALEPIIFTSLFFLGPKLSEQMIHEYKRLMQLLNTCGRLLNDSQSYKREIKQGKLTSLSLFVKEYPAHLIEDATEWIRLTVSESRQNILRNLLQPSEIPRECKQIFWNMVKIIQLFYLHSDEFTSPTAMLEHMQAVLFNPVL

SEQ ID NO: 10 - protein, 922 aa, Artificial Sequence
  fusion protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGGGAAMENKSFGSMCIDDNITRINMLFLEIKRRYSSLSAYDTVHGAYPINLILGLSMVNHLESLGIARHFKLEIKEVLDDVYRFWIENEMVKGMPSASDIMTSSISFKILRWNGYDVSPDVFFSYLRDSDFLPFLENSGQATMALLNLYKASQMMFPGERILEEAKSFSQNYLENIKVGDQSGALKDIEAEVKHALDVPWMAGLERIEHLRYMKAFAFDDIWIGETSSRIPFTGKDLFLSLAKQDFNICQEAQQEDLKALEKWSVDSKLGDLHFARQKLVACYFSAASTLFSPEMSTTRIVWTKNAVLTTLMDDFYDVEGSIEDIQSFVEAVKRWDPIVLCNCSANVKKLFSAIYNTVNDIAQDIWIFQGWDISTQLREIWYELAISMMKEAEWTKTGFIPSLEEYMENGRTSIALEPIIFTSLFFLGPKLSEQMIHEYKRLMQLLNTCGRLLNDSQSYKREIKQGKLNSLSLFVKEYPATSIEDATEWIRLTVSESRQNILRNLLQPSEIPRECKQIFWNMVKIIQLFYLHSDEFTSPTAMLEHMQAVLFNPVL

SEQ ID NO: 11 - protein, 525 aa, Artificial Sequence
  truncated protein
MAKMKENFKREDDKFPTTTTLRSEDIPSNLCIIDTLQRLGVDQFFQYEINTILDNTFRLWQEKHKVIYGNVTTHAMAFRLLRVKGYEVSSEELAPYGNQEAVSQQTNDLPMIIELYRAANERIYEEERSLEKILAWTTIFLNKQVQDNSIPDKKLHKLVEFYLRNYKGITIRLGARRNLELYDMTYYQALKSTNRFSNLCNEDFLVFAKQDFDIHEAQNQKGLQQLQRWYADCRLDTLNFGRDVVIIANYLASLIIGDHAFDYVRLAFAKTSVLVTIMDDFFDCHGSSQECDKIIELVKEWKENPDAEYGSEELEILFMALYNTVNELAERARVEQGRSVKEFLVKLWVEILSAFKIELDTWSNGTQQSFDEYISSSWLSNGSRLTGLLTMQFVGVKLSDEMLMSEECTDLARHVCMVGRLLNDVCSSEREREENIAGKSYSILLATEKDGRKVSEDEAIAEINEMVEYHWRKVLQIVYKKESILPRRCKDVFLEMAKGTFYAYGINDELTSPQQSKEDMKSFVF

SEQ ID NO: 12 - protein, 864 aa, Artificial Sequence
  fusion protein
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGDDDDKIHMNNGKNLNCQLTHKKISKVAEIRVATVNAPPVHDQDDSTENQCHDAVNNIEDPIEYIRTLLRTTGDGRISVSPYDTAWVALIKDLQGRDAPEFPSSLEWIIQNQLADGSWGDAKFFCVYDRLVNTIACVVALRSWDVHAEKVERGVRYINENVEKLRDGNEEHMTCGFEVVFPALLQRAKSLGIQDLPYDAPVIQEIYHSREQKSKRIPLEMMHKVPTSLLFSLEGLENLEWDKLLKLQSADGSFLTSPSSTAFAFMQTRDPKCYQFIKNTIQTFNGGAPHTYPVDVFGRLWAIDRLQRLGISRFFESEIADCIAHIHRFWTEKGVFSGRESEFCDIDDTSMGVRLMRMHGYDVDPNVLKNFKKDDKFSCYGGQMIESPSPIYNLYRASQLRFPGEQILEDANKFAYDFLQEKLAHNQILDKWVISKHLPDEIKLGLEMPWYATLPRVEARYYIQYYAGSGDVWIGKTLYRMPEISNDTYHELAKTDFKRCQAQHQFEWIYMQEWYESCNMEEFGISRKELLVAYFLATASIFELERANERIAWAKSQIISTIIASFFNNQNTSPEDKLAFLTDFKNGNSTNMALVTLTQFLEGFDRYTSHQLKNAWSVWLRKLQQGEGNGGADAELLVNTLNICAGHIAFREEILAHNDYKTLSNLTSKICRQLSQIQNEKELETEGQKTSIKNKELEEDMQRLVKLVLEKSRVGINRDMKKTFLAVVKTYYYKAYHSAQAIDNHMFKVLFEPVA

SEQ ID NO: 13 - protein, 849 aa, Artificial Sequence
  fusion protein
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGDDDDKIHMCRVASLDALNGIQKVGPATIGTPEEENKKIEDSIEYVKELLKTMGDGRISVSPYDTAIVALIKDLEGGDGPEFPSCLEWIAQNQLADGSWGDHFFCIYDRVVNTAACVVALKSWNVHADKIEKGAVYLKENVHKLKDGKIEHMPAGFEFVVPATLERAKALGIKGLPYDDPFIREIYSAKQTRLTKIPKGMIYESPTSLLYSLDGLEGLEWDKILKLQSADGSFITSVSSTAFVFMHTNDLKCHAFIKNALTNCNGGVPHTYPVDIFARLWAVDRLQRLGISRFFEPEIKYLMDHINNVWREKGVFSSRHSQFADIDDTSMGIRLLKMHGYNVNPNALEHFKQKDGKFTCYADQHIESPSPMYNLYRAAQLRFPGEEILQQALQFAYNFLHENLASNHFQEKWVISDHLIDEVRIGLKMPWYATLPRVEASYYLQHYGGSSDVWIGKTLYRMPEISNDTYKILAQLDFNKCQAQHQLEWMSMKEWYQSNNVKEFGISKKELLLAYFLAAATMFEPERTQERIMWAKTQVVSRMITSFLNKENTMSFDLKIALLTQPQHQINGSEMKNGLAQTLPAAFRQLLKEFDKYTRHQLRNTWNKWLMKLKQGDDNGGADAELLANTLNICAGHNEDILSHYEYTALSSLTNKICQRLSQIQDKKMLEIEEGSIKDKEMELEIQTLVKLVLQETSGGIDRNIKQTFLSVFKTFYYRAYHDAKTIDAHIFQVLFEPVV

SEQ ID NO: 14 - protein, 920 aa, Artificial Sequence
  fusion protein
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGDDDDKIHMQVIITSSHRFFCHHLHQLKSPTSLSAQKAEFKKHGPRNWLFQTEGSLLYKPVRLNCATSDASYLGNVNEYLESDHSKNSEEKDIQVSRTIQMKGLTEEIKHMLNSMEDGRLNVLAYDTAWVSFIPNTTNNGNDQRPMFPSCLQWIIDNQLSDGSWGEEIVFCIYDRLLNTLVCVIALTLWNTCLHKRNKGVMFIKENLSKLETGEVENMTSGFELVFPTLLEKAQQLDIDIPYDAPVLKDIYARREVKLTRIPKDVIHTIPTTVLFSLEGLRDDLDWQRLLKLQMPDGSFLISPASTAFAFMETNDEKCLAYLQNVVEKSNGGARQYPFDLVTRLWAIDRLQRLGISYYFAEEFKELLNHVFRYWDEENGIFSGRNSNVSDVDDTCMAIRLLRLHGYDVSPDALNNFKDGDQFVCFRGEVDGSPTHMFNLYRCSQVLFPGEKILEEAKNFTYNFLQQCLANNRCLDKWVIAKDIPGEIWYALEFPWYASLPRVEARYYIEQYGGADDIWIGKTLYRMPDVNNNVYLQAAKLDYNRCQSQHRFEWLIMQEWFEKCNFQQFGISKKYLLVSYFLAAASIFEVEKSRERLAWAKSRIICKMITSYYNDEATTWTTRNSLLMEFKVSHDPTRKNGNETKEILVLKNLRQFLRQLSEETFEDLGKDIHHQLQNAWETWLVFLREEKNACQEETELLVRTINLSGGYMTHDEILFDADYENLSNLTNKVCGKLNELQNDKVTGGSKNTNIELDMQALVKLVFGNTSSNINQDIKQTFFAVVKTFYYSAHVSEEIMNFHISKVLFQQV

SEQ ID NO: 15 - protein, 366 aa, Cupressus gigantea
MSSFDAHDLDLDKFPEVVRDRLTQFLDAQELTIADIGAPVTDAVAHLRSFVLNGGKRIRPLYAWAGFLAAQGHKNSSEKLESVLDAAASLEFIQACALIHDDIIDSSDTRRGAPTVHRAVEADHRANNFEGDPEHFGVSVSILAGDMALVWAEDMLQDSGLSAEALARTRDAWRGMRTEVIGGQLLDIYLESHANESVELADSVNRFKTAAYTIARPLHLGASIAGGSPQLIDALLHYGHDIGIAFQLRDDLLGVFGDPAITGKPAGDDIREGKRTVLLALALQRADKQSPEAATAIRAGVGKVTSPEDIAVITEHIRATGAEEEVEQRISQLTESGLAHLDDVDIPDEVRAQLRALAIRSTERRM

SEQ ID NO: 16 - DNA, 2769 bp, Artificial Sequence
  fusion
atgaagatcgaggaaggcaagctcgtgatctggatcaacggcgacaagggctacaacggcctggccgaggtgggcaagaagttcgagaaggacaccggcatcaaggtgacggtggagcacccggacaagctcgaggagaagttcccgcaggtggcggccacgggcgacggcccggacatcatcttctgggcccatgaccgcttcggcggctacgcccagtcgggcctgctggccgagatcaccccggacaaggcgttccaggacaagctctatcccttcacgtgggacgccgtgcgctacaacggcaagctgatcgcgtatcccatcgcggtggaggccctgtcgctcatctataacaaggacctgctcccgaacccgcccaagacctgggaggagatccccgccctcgacaaggagctgaaggccaagggcaagtcggcgctcatgttcaacctgcaggagccgtacttcacctggcccctgatcgcggccgacggcggctacgcgttcaagtatgagaacggcaagtatgacatcaaggacgtgggcgtggacaacgcgggcgccaaggccggcctgaccttcctcgtggacctgatcaagaacaagcacatgaacgccgacacggactactcgatcgcggaggccgcgttcaacaagggcgagactgccatgacgatcaacggcccgtgggcgtggtcgaacatcgacacctcgaaggtgaactatggcgtgaccgtgctccccacgttcaagggccagccctcgaagcccttcgtgggcgtgctgtcggcgggcatcaacgccgcgtcgccgaacaaggagctcgcgaaggagttcctggagaactacctgctcaccgacgagggcctggaggccgtgaacaaggacaagcccctgggcgccgtggccctgaagtcgtatgaggaagagctggtgaaggacccgcgcatcgcggccaccatggagaacgcgcagaagggcgagatcatgccgaacatcccccagatgtcggccttctggtatgcggtgcgcaccgccgtgatcaacgcggcctcgggccgccagaccgtggacgaggccctcaaggacgcccagaccggcggaggtgctgccatggagaacaagtcgttcggctcgatgtgcatcgacgacaacatcacccgcatcaacatgctcttcctcgagatcaagcgccgctactcgtcgctctcggcctacgacaccgtgcacggcgcctacccgatcaacctcatcctcggcctctcgatggtgaaccacctcgagtcgctcggcatcgcccgccacttcaagctcgagatcaaggaggtgctcgacgacgtgtaccgcttctggatcgagaacgagatggtgaagggcatgccgtcggcctcggacatcatgacctcgtcgatctcgttcaagatcctccgctggaacggctacgacgtgtcgccggacgtgttcttctcgtacctccgcgactcggacttcctcccgttcctcgagaactcgggccaggccactatggccctcctcaacctctacaaggcctcgcagatgatgttcccgggcgagcgcatcctcgaggaggccaagtcgttctcgcagaactacctcgagaacatcaaggtgggcgaccagtcgggcgccctcaaggacatcgaggccgaggtgaagcacgccctcgacgtgccgtggatggccggcctcgagcgcatcgagcacctccgctacatgaaggccttcgccttcgacgacatctggatcggcgagacgtcgtcgcgcatcccgttcaccggcaaggacctcttcctctcgctcgccaagcaggacttcaacatctgccaggaggcccagcaggaggacctcaaggccctcgagaagtggtcggtggactcgaagctcggcgacctccacttcgcccgccagaagctcgtggcctgctacttctcggccgcctcgaccctcttctcgccggagatgtcgaccacccgcatcgtgtggaccaagaacgccgtgctcaccaccctcatggacgacttctacgacgtggagggctcgatcgaggacatccagtcgttcgtggaggccgtgaagcgctgggacccgatcgtgctctgcaactgctcggccaacgtgaagaagctcttctcggccatctacaacaccgtgaacgacatcgcccaggacatctggatcttccagggctgggacatctcgacccagctccgcgagatctggtacgagctcgccatctcgatgatgaaggaggccgagtggaccaagaccggcttcatcccgtcgctcgaggagtacatggagaacggccgcacctcgatcgccctcgagccgatcatcttcacctcgctcttcttcctcggcccgaagctctcggagcagatgatccacgagtacaagcgcctcatgcagctcctcaacacctgcggcaggctcctcaacgactcgcagtcgtacaagcgcgagatcaagcagggcaagctcaactcgctctcgctcttcgtgaaggagtacccggccacctcgatcgaggacgccaccgagtggatcaggctcaccgtgtcggagtcgcgccagaacatcctccgcaacctcctccagccgtcggagatcccgcgcgagtgcaagcagatcttctggaacatggtgaagatcatccagctcttctacctccactcggacgagttcacctcgccgaccgccatgctcgagcacatgcaggccgtgctcttcaacccggtgctctaa

SEQ ID NO: 17 - DNA, 2769 bp, Artificial Sequence
  fusion
atgaagatcgaggaaggcaagctcgtgatctggatcaacggcgacaagggctacaacggcctggccgaggtgggcaagaagttcgagaaggacaccggcatcaaggtgacggtggagcacccggacaagctcgaggagaagttcccgcaggtggcggccacgggcgacggcccggacatcatcttctgggcccatgaccgcttcggcggctacgcccagtcgggcctgctggccgagatcaccccggacaaggcgttccaggacaagctctatcccttcacgtgggacgccgtgcgctacaacggcaagctgatcgcgtatcccatcgcggtggaggccctgtcgctcatctataacaaggacctgctcccgaacccgcccaagacctgggaggagatccccgccctcgacaaggagctgaaggccaagggcaagtcggcgctcatgttcaacctgcaggagccgtacttcacctggcccctgatcgcggccgacggcggctacgcgttcaagtatgagaacggcaagtatgacatcaaggacgtgggcgtggacaacgcgggcgccaaggccggcctgaccttcctcgtggacctgatcaagaacaagcacatgaacgccgacacggactactcgatcgcggaggccgcgttcaacaagggcgagactgccatgacgatcaacggcccgtgggcgtggtcgaacatcgacacctcgaaggtgaactatggcgtgaccgtgctccccacgttcaagggccagccctcgaagcccttcgtgggcgtgctgtcggcgggcatcaacgccgcgtcgccgaacaaggagctcgcgaaggagttcctggagaactacctgctcaccgacgagggcctggaggccgtgaacaaggacaagcccctgggcgccgtggccctgaagtcgtatgaggaagagctggtgaaggacccgcgcatcgcggccaccatggagaacgcgcagaagggcgagatcatgccgaacatcccccagatgtcggccttctggtatgcggtgcgcaccgccgtgatcaacgcggcctcgggccgccagaccgtggacgaggccctcaaggacgcccagaccggcggaggtgctgccatggagaacaagtcgttcggctcgatgtgcatcgacgacaacatcacccgcatcaacatgctcttcctcgagatcaagcgccgctactcgtcgctctcggcctacgacaccgtgcacggcgcctacccgatcaacctcatcctcggcctctcgatggtgaaccacctcgagtcgctcggcatcgcccgccacttcaagctcgagatcaaggaggtgctcgacgacgtgtaccgcttctggatcgagaacgagatggtgaagggcatgccgtcggcctcggacatcatgacctcgtcgatctcgttcaagatcctccgctggaacggctacgacgtgtcgccggacgtgttcttctcgtacctccgcgactcggacttcctcccgttcctcgagaactcgggccaggccactatggccctcctcaacctctacaaggcctcgcagatgatgttcccgggcgagcgcatcctcgaggaggccaagtcgttctcgcagaactacctcgagaacatcaaggtgggcgaccagtcgggcgccctcaaggacatcgaggccgaggtgaagcacgccctcgacgtgccgtggatggccggcctcgagcgcatcgagcacctccgctacatgaaggccttcgccttcgacgacatctggatcggcgagacgtcgtcgcgcatcccgttcaccggcaaggacctcttcctctcgctcgccaagcaggacttcaacatctgccaggaggcccagcaggaggacctcaaggccctcgagaagtggtcggtggactcgaagctcggcgacctccacttcgcccgccagaagctcgtggcctgctacttctcggccgcctcgaccctcttctcgccggagatgtcgaccacccgcatcgtgtggaccaagaacgccgtgctcaccaccctcatggacgacttctacgacgtggagggctcgatcgaggacatccagtcgttcgtggaggccgtgaagcgctgggacccgatcgtgctctgcaactgctcggccaacgtgaagaagctcttctcggccatctacaacaccgtgaacgacatcgcccaggacatctggatcttccagggctgggacatctcgacccagctccgcgagatctggtacgagctcgccatctcgatgatgaaggaggccgagtggaccaagaccggcttcatcccgtcgctcgaggagtacatggagaacggccgcacctcgatcgccctcgagccgatcatcttcacctcgctcttcttcctcggcccgaagctctcggagcagatgatccacgagtacaagcgcctcatgcagctcctcaacacctgcggcaggctcctcaacgactcgcagtcgtacaagcgcgagatcaagcagggcaagctcacctcgctctcgctcttcgtgaaggagtacccggcccacctcatcgaggacgccaccgagtggatcaggctcaccgtgtcggagtcgcgccagaacatcctccgcaacctcctccagccgtcggagatcccgcgcgagtgcaagcagatcttctggaacatggtgaagatcatccagctcttctacctccactcggacgagttcacctcgccgaccgccatgctcgagcacatgcaggccgtgctcttcaacccggtgctctaa

SEQ ID NO: 18 - DNA, 2766 bp, Artificial Sequence
  fusion
atgaagatcgaggaaggcaagctcgtgatctggatcaacggcgacaagggctacaacggcctggccgaggtgggcaagaagttcgagaaggacaccggcatcaaggtgacggtggagcacccggacaagctcgaggagaagttcccgcaggtggcggccacgggcgacggcccggacatcatcttctgggcccatgaccgcttcggcggctacgcccagtcgggcctgctggccgagatcaccccggacaaggcgttccaggacaagctctatcccttcacgtgggacgccgtgcgctacaacggcaagctgatcgcgtatcccatcgcggtggaggccctgtcgctcatctataacaaggacctgctcccgaacccgcccaagacctgggaggagatccccgccctcgacaaggagctgaaggccaagggcaagtcggcgctcatgttcaacctgcaggagccgtacttcacctggcccctgatcgcggccgacggcggctacgcgttcaagtatgagaacggcaagtatgacatcaaggacgtgggcgtggacaacgcgggcgccaaggccggcctgaccttcctcgtggacctgatcaagaacaagcacatgaacgccgacacggactactcgatcgcggaggccgcgttcaacaagggcgagactgccatgacgatcaacggcccgtgggcgtggtcgaacatcgacacctcgaaggtgaactatggcgtgaccgtgctccccacgttcaagggccagccctcgaagcccttcgtgggcgtgctgtcggcgggcatcaacgccgcgtcgccgaacaaggagctcgcgaaggagttcctggagaactacctgctcaccgacgagggcctggaggccgtgaacaaggacaagcccctgggcgccgtggccctgaagtcgtatgaggaagagctggtgaaggacccgcgcatcgcggccaccatggagaacgcgcagaagggcgagatcatgccgaacatcccccagatgtcggccttctggtatgcggtgcgcaccgccgtgatcaacgcggcctcgggccgccagaccgtggacgaggccctcaaggacgcccagaccggcggaggtgctgccatggagaagaagtcgttcggctcgatgtgcatcgacgacgacatcaccaagatcaagatgctcttctcggagatcaagcgccgctactcgtcgctctcggcctacgacaccgtgcacggcgcctacccgatcaacctcatcctcggcctctcgatggtgaaccacctcgagtcgctcggcatcgcccgccacttcaagctcgagatcaaggaggtgctcgacgacgtgtaccgcttctggatcgagaacgagatggtggagggcatgccgtcggcctcggacatcatgacctcgtcgatctcgttccgcatcctccgctggaacggctacgacgtgtcgccggacgtgttcttctcgtacctccgcgactcggacttcctcctcttcctcgagaactcggaccaggccaccatcgccctcctcaacctctacaaggcctcgcagatgatgttcccgggcgagcgcatcctcgaggaggccaagctcttctcgcagaactacctcgagaacatcaagatgggcaaccagtcgggcgccctcaaggacatcgaggccgaggtgaagcacgccctcgacgtgccgtggatggccaacctcgagcgcatcgagcacctccgctacatgaaggccttcgccttcgacaacatctggatcggcgagacgtcgtcgcgcatcccgttcaccggcaaggacctcttcctctcgttcgccaagcaggacttcaacatctgccaggccgcccagcaggaggacctcaaggccctcgagaagtggtcggtggactcgaagctcggcgacctccacttcgcccgccagaagctcgtggcctgctacttctcggccgcctcgatcctcttctcgccggagatgtcggccacccgcatcgtgtggaccaagaacgccgtgctcatcaccctcatggacgacttctacgacgtggagggctcgatcgaggacatccagtcgttcgtggaggccgtgaagcgctgggacccgatcgtgctctgcaactgctcggccaacgtgaagatcctcttctcggccatctacaacaccgtgaacgacatcgcccaggacatctggatcttccagggctgggacatctcgacccagctccgcgagatctggtacgagctcgccatctcgatgatgaaggaggccgagtggaccaagaccggcttcatcccgtcgctcgaggagtacatggagaacggccgcacctcgatcgccctcgagccgatcatcttcacctcgctcttcttcatcggcccgaagctctcggagcagatgatccaccaccacgagtacaagcgcctcctccagctcatgaacacctgcggccgcctcctcaacgacatccagtcggacaagcgcgagatgaagcagggcaagctcacctcggtgtcgctcttcgtgaaggagtacccgatcgaggacgccaccaagtggatccgcctcaccgtgtcggagaacacccagaacatcctcaagaagatcctccagccgtcggagatcccgcgcgagtgcaagcagatcttctggaacatggtgaagatcatccagctcttctgcctccactcggacgagttcacctcgccgaccaccatgctcgagcacatgaaggccgtgctcttctacccggtgctctga

SEQ ID NO: 19 - DNA, 1578 bp, Salvia sclarea
atggccaagatgaaggagaacttcaagcgcgaggacgacaagttcccgaccaccaccaccctccgctcggaggacatcccgtcgaacctctgcatcatcgacaccctccagcgcctcggcgtggaccagttcttccagtacgagatcaacaccatcctcgacaacaccttccgcctctggcaggagaagcacaaggtgatctacggcaacgtgaccacccacgccatggccttccgcctcctccgcgtgaagggctacgaggtgtcgtcggaggagctcgccccgtacggcaaccaggaggccgtgtcgcagcagaccaacgacctcccgatgatcatcgagctctaccgcgccgccaacgagcgcatctacgaggaggagcgctcgctcgagaagatcctcgcctggaccaccatcttcctcaacaagcaggtgcaggacaactcgatcccggacaagaagctccacaagctcgtggagttctacctccgcaactacaagggcatcaccatccgcctcggcgcccgccgcaacctcgagctctacgacatgacctactaccaggccctcaagtcgaccaaccgcttctcgaacctctgcaacgaggacttcctcgtgttcgccaagcaggacttcgacatccacgaggcccagaaccagaagggcctccagcagctccagcgctggtacgccgactgccgcctcgacaccctcaacttcggccgcgacgtggtgatcatcgccaactacctcgcctcgctcatcatcggcgaccacgccttcgactacgtgcgcctcgccttcgccaagacctcggtgctcgtgaccatcatggacgacttcttcgactgccacggctcgtcgcaggagtgcgacaagatcatcgagctcgtgaaggagtggaaggagaacccggacgccgagtacggctcggaggagctcgagatcctcttcatggccctctacaacaccgtgaacgagctcgccgagcgcgcccgcgtggagcagggccgctcggtgaaggagttcctcgtgaagctctgggtggagatcctctcggccttcaagatcgagctcgacacctggtcgaacggcacccagcagtcgttcgacgagtacatctcgtcgtcgtggctctcgaacggctcgcgcctcaccggcctcctcaccatgcagttcgtgggcgtgaagctctcggacgagatgctcatgtcggaggagtgcaccgacctcgcccgccacgtgtgcatggtgggccgcctcctcaacgacgtgtgctcgtcggagcgcgagcgcgaggagaacatcgccggcaagtcgtactcgatcctcctcgccaccgagaaggacggccgcaaggtgtcggaggacgaggccatcgccgagatcaacgagatggtggagtaccactggcgcaaggtgctccagatcgtgtacaagaaggagtcgatcctcccgcgccgctgcaaggacgtgttcctcgagatggccaagggcaccttctacgcctacggcatcaacgacgagctcacctcgccgcagcagtcgaaggaggacatgaagtcgttcgtgttctga

SEQ ID NO: 20 - DNA, 2550 bp, Artificial Sequence
  fusion
atgtcggacaagatcatccacctgaccgacgacagcttcgacaccgacgtgctgaaggccgacggcgccatcctcgtcgatttctgggccgaatggtgcggcccctgcaagatgatcgcgccgatcctcgacgagatcgccgacgaatatcagggcaagctgaccgtcgccaagctgaacatcgaccagaacccgggcacggcgccgaaatacggcatccgcggcatcccgacgctgctgctcttcaagaacggcgaggtggcggccaccaaggtcggcgcgctgtcgaagggccagctgaaggagttcctcgatgcgaacctcgccggtggtgatgacgacgacaagattcatatgtgccgcgtggcctcgctcgacgccctcaacggcatccagaaggtgggcccggccaccatcggcaccccggaggaggagaacaagaagatcgaggactcgatcgagtacgtgaaggagctcctcaagaccatgggcgacggccgcatctcggtgtcgccgtacgacaccgccatcgtggccctcatcaaggacctcgagggcggcgacggcccggagttcccgtcgtgcctcgagtggatcgcccagaaccagctcgccgacggctcgtggggcgaccacttcttctgcatctacgaccgcgtggtgaacaccgccgcctgcgtggtggccctcaagtcgtggaacgtgcacgccgacaagatcgagaagggcgccgtgtacctcaaggagaacgtgcacaagctcaaggacggcaagatcgagcacatgccggccggcttcgagttcgtggtgccggccaccctcgagcgcgccaaggccctcggcatcaagggcctcccgtacgacgacccgttcatccgcgagatctactcggccaagcagacccgcctcaccaagatcccgaagggcatgatctacgagtcgccgacctcgctcctctactcgctcgacggcctcgagggcctcgagtgggacaagatcctcaagctccagtcggccgacggctcgttcatcacctcggtgtcgtcgaccgccttcgtgttcatgcacaccaacgacctcaagtgccacgccttcatcaagaacgccctcaccaactgcaacggcggcgtgccgcacacctacccggtggacatcttcgcccgcctctgggccgtggaccgcctccagcgcctcggcatctcgcgcttcttcgagccggagatcaagtacctcatggaccacatcaacaacgtgtggcgcgagaagggcgtgttctcgtcgcgccactcgcagttcgccgacatcgacgacacctcgatgggcatccgcctcctcaagatgcacggctacaacgtgaacccgaacgccctcgagcacttcaagcagaaggacggcaagttcacctgctacgccgaccagcacatcgagtcgccgtcgccgatgtacaacctctaccgcgccgcccagctccgcttcccgggcgaggagatcctccagcaggccctccagttcgcctacaacttcctccacgagaacctcgcctcgaaccacttccaggagaagtgggtgatctcggaccacctcatcgacgaggtgcgcatcggcctcaagatgccgtggtacgccaccctcccgcgcgtggaggcctcgtactacctccagcactacggcggctcgtcggacgtgtggatcggcaagaccctctaccgcatgccggagatctcgaacgacacctacaagatcctcgcccagctcgacttcaacaagtgccaggcccagcaccagctcgagtggatgtcgatgaaggagtggtaccagtcgaacaacgtgaaggagttcggcatctcgaagaaggagctcctcctcgcctacttcctcgccgccgccaccatgttcgagccggagcgcacccaggagcgcatcatgtgggccaagacccaggtggtgtcgcgcatgatcacctcgttcctcaacaaggagaacaccatgtcgttcgacctcaagatcgccctcctcacccagccgcagcaccagatcaacggctcggagatgaagaacggcctcgcccagaccctcccggccgccttccgccagctcctcaaggagttcgacaagtacacccgccaccagctccgcaacacctggaacaagtggctcatgaagctcaagcagggcgacgacaacggcggcgccgacgccgagctcctcgccaacaccctcaacatctgcgccggccacaacgaggacatcctctcgcactacgagtacaccgccctctcgtcgctcaccaacaagatctgccagcgcctctcgcagatccaggacaagaagatgctcgagatcgaggagggctcgatcaaggacaaggagatggagctcgagatccagaccctcgtgaagctcgtgctccaggagacgtcgggcggcatcgaccgcaacatcaagcagaccttcctctcggtgttcaagaccttctactaccgcgcctaccacgacgccaagaccatcgacgcccacatcttccaggtgctcttcgagccggtggtgtga

SEQ ID NO: 21 - DNA, 2595 bp, Artificial Sequence
  fusion
atgtcggacaagatcatccacctgaccgacgacagcttcgacaccgacgtgctgaaggccgacggcgccatcctcgtcgatttctgggccgaatggtgcggcccctgcaagatgatcgcgccgatcctcgacgagatcgccgacgaatatcagggcaagctgaccgtcgccaagctgaacatcgaccagaacccgggcacggcgccgaaatacggcatccgcggcatcccgacgctgctgctcttcaagaacggcgaggtggcggccaccaaggtcggcgcgctgtcgaagggccagctgaaggagttcctcgatgcgaacctcgccggtggtgatgacgacgacaagattcatatgaacaacggcaagaacctcaactgccagctcacccacaagaagatctcgaaggtggccgagatccgcgtggccaccgtgaacgccccgccggtgcacgaccaggacgactcgaccgagaaccagtgccacgacgccgtgaacaacatcgaggacccgatcgagtacatccgcaccctcctccgcaccaccggcgacggccgcatctcggtgtcgccgtacgacaccgcctgggtggccctcatcaaggacctccagggccgcgacgccccggagttcccgtcgtcgctcgagtggatcatccagaaccagctcgccgacggctcgtggggcgacgccaagttcttctgcgtgtacgaccgcctcgtgaacaccatcgcctgcgtggtggccctccgctcgtgggacgtgcacgccgagaaggtggagcgcggcgtgcgctacatcaacgagaacgtggagaagctccgcgacggcaacgaggagcacatgacctgcggcttcgaggtggtgttcccggccctcctccagcgcgccaagtcgctcggcatccaggacctcccgtacgacgccccggtgatccaggagatctaccactcgcgcgagcagaagtcgaagcgcatcccgctcgagatgatgcacaaggtgccgacctcgctcctcttctcgctcgagggcctcgagaacctcgagtgggacaagctcctcaagctccagtcggccgacggctcgttcctcacctcgccgtcgtcgaccgccttcgccttcatgcagacccgcgacccgaagtgctaccagttcatcaagaacaccatccagaccttcaacggcggcgccccgcacacctacccggtggacgtgttcggccgcctctgggccatcgaccgcctccagcgcctcggcatctcgcgcttcttcgagtcggagatcgccgactgcatcgcccacatccaccgcttctggaccgagaagggcgtgttctcgggccgcgagtcggagttctgcgacatcgacgacacctcgatgggcgtgcgcctcatgcgcatgcacggctacgacgtggacccgaacgtgctcaagaacttcaagaaggacgacaagttctcgtgctacggcggccagatgatcgagtcgccgtcgccgatctacaacctctaccgcgcctcgcagctccgcttcccgggcgagcagatcctcgaggacgccaacaagttcgcctacgacttcctccaggagaagctcgcccacaaccagatcctcgacaagtgggtgatctcgaagcacctcccggacgagatcaagctcggcctcgagatgccgtggtacgccaccctcccgcgcgtggaggcccgctactacatccagtactacgccggctcgggcgacgtgtggatcggcaagaccctctaccgcatgccggagatctcgaacgacacctaccacgagctcgccaagaccgacttcaagcgctgccaggcccagcaccagttcgagtggatctacatgcaggagtggtacgagtcgtgcaacatggaggagttcggcatctcgcgcaaggagctcctcgtggcctacttcctcgccaccgcctcgatcttcgagctcgagcgcgccaacgagcgcatcgcctgggccaagtcgcagatcatctcgaccatcatcgcctcgttcttcaacaaccagaacacctcgccggaggacaagctcgccttcctcaccgacttcaagaacggcaactcgaccaacatggccctcgtgaccctcacccagttcctcgagggcttcgaccgctacacctcgcaccagctcaagaacgcctggtcggtgtggctccgcaagctccagcagggcgagggcaacggcggcgccgacgccgagctcctcgtgaacaccctcaacatctgcgccggccacatcgccttccgcgaggagatcctcgcccacaacgactacaagaccctctcgaacctcacctcgaagatctgccgccagctctcgcagatccagaacgagaaggagctcgagacggagggccagaagacctcgatcaagaacaaggagctcgaggaggacatgcagcgcctcgtgaagctcgtgctcgagaagtcgcgcgtgggcatcaaccgcgacatgaagaagaccttcctcgccgtggtgaagacctactactacaaggcctaccactcggcccaggccatcgacaaccacatgttcaaggtgctcttcgagccggtggcctga

SEQ ID NO: 22 - DNA, 1101 bp, Cupressus gigantea
atgtcgtcgttcgacgcccacgacctcgacctcgacaagttcccggaggtggtgcgcgaccgcctcacccagttcctcgacgcccaggagctcaccatcgccgacatcggcgccccggtgaccgacgccgtggcccacctccgctcgttcgtgctcaacggcggcaagcgcatccgcccgctctacgcctgggccggcttcctcgccgcccagggccacaagaactcgtcggagaagctcgagtcggtgctcgacgccgccgcctcgctcgagttcatccaggcctgcgccctcatccacgacgacatcatcgactcgtcggacacccgccgcggcgccccgaccgtgcaccgcgccgtggaggccgaccaccgcgccaacaacttcgagggcgacccggagcacttcggcgtgtcggtgtcgatcctcgccggcgacatggccctcgtgtgggccgaggacatgctccaggactcgggcctctcggccgaggccctcgcccgcacccgcgacgcctggcgcggcatgcgcaccgaggtgatcggcggccagctcctcgacatctacctcgagtcgcacgccaacgagtcggtggagctcgccgactcggtgaaccgcttcaagaccgccgcctacaccatcgcccgcccgctccacctcggcgcctcgatcgccggcggctcgccgcagctcatcgacgccctcctccactacggccacgacatcggcatcgccttccagctccgcgacgacctcctcggcgtgttcggcgacccggccatcaccggcaagccggccggcgacgacatccgcgagggcaagcgcaccgtgctcctcgccctcgccctccagcgcgccgacaagcagtcgccggaggccgccaccgccatccgcgccggcgtgggcaaggtgacctcgccggaggacatcgccgtgatcaccgagcacatccgcgccaccggcgccgaggaggaggtggagcagcgcatctcgcagctcaccgagtcgggcctcgcccacctcgacgacgtggacatcccggacgaggtgcgcgcccagctccgcgccctcgccatccgctcgaccgagcgccgcatgtga

SEQ ID NO: 23 - DNA, 2763 bp, Artificial Sequence
  fusion
atgtcggacaagatcatccacctgaccgacgacagcttcgacaccgacgtgctgaaggccgacggcgccatcctcgtcgatttctgggccgaatggtgcggcccctgcaagatgatcgcgccgatcctcgacgagatcgccgacgaatatcagggcaagctgaccgtcgccaagctgaacatcgaccagaacccgggcacggcgccgaaatacggcatccgcggcatcccgacgctgctgctcttcaagaacggcgaggtggcggccaccaaggtcggcgcgctgtcgaagggccagctgaaggagttcctcgatgcgaacctcgccggtggtgatgacgacgacaagattcatatgcaggtgatcatcacctcgtcgcaccgcttcttctgccaccacctccaccagctcaagtcgccgacctcgctctcggcccagaaggccgagttcaagaagcacggcccgcgcaactggctcttccagaccgagggctcgctcctctacaagccggtgcgcctcaactgcgccacctcggacgcctcgtacctcggcaacgtgaacgagtacctcgagtcggaccactcgaagaactcggaggagaaggacatccaggtgtcgcgcaccatccagatgaagggcctcaccgaggagatcaagcacatgctcaactcgatggaggacggccgcctcaacgtgctcgcctacgacaccgcctgggtgtcgttcatcccgaacaccaccaacaacggcaacgaccagcgcccgatgttcccgtcgtgcctccagtggatcatcgacaaccagctctcggacggctcgtggggcgaggagatcgtgttctgcatctacgaccgcctcctcaacaccctcgtgtgcgtgatcgccctcaccctctggaacacctgcctccacaagcgcaacaagggcgtgatgttcatcaaggagaacctctcgaagctcgagacgggcgaggtggagaacatgacctcgggcttcgagctcgtgttcccgaccctcctcgagaaggcccagcagctcgacatcgacatcccgtacgacgccccggtgctcaaggacatctacgcccgccgcgaggtgaagctcacccgcatcccgaaggacgtgatccacaccatcccgaccaccgtgctcttctcgctcgagggcctccgcgacgacctcgactggcagcgcctcctcaagctccagatgccggacggctcgttcctcatctcgccggcctcgaccgccttcgccttcatggagacgaacgacgagaagtgcctcgcctacctccagaacgtggtggagaagtcgaacggcggcgcccgccagtacccgttcgacctcgtgacccgcctctgggccatcgaccgcctccagcgcctcggcatctcgtactacttcgccgaggagttcaaggagctcctcaaccacgtgttccgctactgggacgaggagaacggcatcttctcgggccgcaactcgaacgtgtcggacgtggacgacacctgcatggccatccgcctcctccgcctccacggctacgacgtgtcgccggacgccctcaacaacttcaaggacggcgaccagttcgtgtgcttccgcggcgaggtggacggctcgccgacccacatgttcaacctctaccgctgctcgcaggtgctcttcccgggcgagaagatcctcgaggaggccaagaacttcacctacaacttcctccagcagtgcctcgccaacaaccgctgcctcgacaagtgggtgatcgccaaggacatcccgggcgagatctggtacgccctcgagttcccgtggtacgcctcgctcccgcgcgtggaggcccgctactacatcgagcagtacggcggcgccgacgacatctggatcggcaagaccctctaccgcatgccggacgtgaacaacaacgtgtacctccaggccgccaagctcgactacaaccgctgccagtcgcagcaccgcttcgagtggctcatcatgcaggagtggttcgagaagtgcaacttccagcagttcggcatctcgaagaagtacctcctcgtgtcgtacttcctcgccgccgcctcgatcttcgaggtggagaagtcgcgcgagcgcctcgcctgggccaagtcgcgcatcatctgcaagatgatcacctcgtactacaacgacgaggccaccacctggaccacccgcaactcgctcctcatggagttcaaggtgtcgcacgacccgacccgcaagaacggcaacgagacgaaggagatcctcgtgctcaagaacctccgccagttcctccgccagctctcggaggagacgttcgaggacctcggcaaggacatccaccaccagctccagaacgcctgggagacgtggctcgtgttcctccgcgaggagaagaacgcctgccaggaggagacggagctcctcgtgcgcaccatcaacctctcgggcggctacatgacccacgacgagatcctcttcgacgccgactacgagaacctctcgaacctcaccaacaaggtgtgcggcaagctcaacgagctccagaacgacaaggtgaccggcggctcgaagaacaccaacatcgagctcgacatgcaggccctcgtgaagctcgtgttcggcaacacctcgtcgaacatcaaccaggacatcaagcagaccttcttcgccgtggtgaagaccttctactactcggcccacgtgtcggaggagatcatgaacttccacatctcgaaggtgctcttccagcaggtgtga

SEQ ID NO: 24 - protein, 12 aa, Artificial Sequence
  domain
GRTSIALEPIIF

SEQ ID NO: 25 - protein, 12 aa, Artificial Sequence
  domain
SWVSIGCRICIL

SEQ ID NO: 26 - protein, 12 aa, Artificial Sequence
  domain
SWLSNGSRLTGL

SEQ ID NO: 27 - protein, 12 aa, Artificial Sequence
  domain
SWLSIGSRLTGL

SEQ ID NO: 28 - protein, 940 aa, Artificial Sequence
  fusion protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGGGAAMLSVEDSSNVLIKAEKENREKKSFGSMCIDDNITRINMLFLEIKRRYSSLSAYDTVHGAYPINLILGLSMVNHLESLGIARHFKLEIKEVLDDVYRFWIENEMVKGMPSASDIMTSSISFKILRWNGYDVSPDVFFSYLRDSDFLPFLENSGQATMALLNLYKASQMMFPGERILEEAKSFSQNYLENIKVGDQSGALKDIEAEVKHALDVPWMAGLERIEHLRYMKAFAFDDIWIGETSSRIPFTGKDLFLSLAKQDFNICQEAQQEDLKALEKWSVDSKLGDLHFARQKLVACYFSAASTLFSPEMSTTRIVWTKNAVLTTLMDDFYDVEGSIEDIQSFVEAVKRWDPIVLCNCSANVKKLFSAIYNTVNDIAQDIWIFQGWDISTQLREIWYELAISMMKEAEWTKTGFIPSLEEYMENGRTSIALEPIIFTSLFFLGPKLSEQMIHEYKRLMQLLNTCGRLLNDSQSYKREIKQGKLNSLSLFVKEYPATSIEDATEWIRLTVSESRQNILRNLLQPSEIPRECKQIFWNMVKIIQLFYLHSDEFTSPTAMLEHMQAVLFNPVL

SEQ ID NO: 29 - protein, 964 aa, Artificial Sequence
  fusion protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGGGAAMAGASNFNFNGRKKFNPRTHQKVKSLSVEDSSNVLIKAEKENREKKSFGSMCIDDNITRINMLFLEIKRRYSSLSAYDTVHGAYPINLILGLSMVNHLESLGIARHFKLEIKEVLDDVYRFWIENEMVKGMPSASDIMTSSISFKILRWNGYDVSPDVFFSYLRDSDFLPFLENSGQATMALLNLYKASQMMFPGERILEEAKSFSQNYLENIKVGDQSGALKDIEAEVKHALDVPWMAGLERIEHLRYMKAFAFDDIWIGETSSRIPFTGKDLFLSLAKQDFNICQEAQQEDLKALEKWSVDSKLGDLHFARQKLVACYFSAASTLFSPEMSTTRIVWTKNAVLTTLMDDFYDVEGSIEDIQSFVEAVKRWDPIVLCNCSANVKKLFSAIYNTVNDIAQDIWIFQGWDISTQLREIWYELAISMMKEAEWTKTGFIPSLEEYMENGRTSIALEPIIFTSLFFLGPKLSEQMIHEYKRLMQLLNTCGRLLNDSQSYKREIKQGKLNSLSLFVKEYPATSIEDATEWIRLTVSESRQNILRNLLQPSEIPRECKQIFWNMVKIIQLFYLHSDEFTSPTAMLEHMQAVLFNPVL

SEQ ID NO: 30 - protein, 964 aa, Artificial Sequence
  fusion protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGGGAAMAGASNFNFNGRKKFNPRTHQKVKSLSRRDSSNVLIKWEKENREKKSFGSMCIDDNITRINMLFLEIKRRYSSLSAYDTVHGAYPINLILGLSMVNHLESLGIARHFKLEIKEVLDDVYRFWIENEMVKGMPSASDIMTSSISFKILRWNGYDVSPDVFFSYLRDSDFLPFLENSGQATMALLNLYKASQMMFPGERILEEAKSFSQNYLENIKVGDQSGALKDIEAEVKHALDVPWMAGLERIEHLRYMKAFAFDDIWIGETSSRIPFTGKDLFLSLAKQDFNICQEAQQEDLKALEKWSVDSKLGDLHFARQKLVACYFSAASTLFSPEMSTTRIVWTKNAVLTTLMDDFYDVEGSIEDIQSFVEAVKRWDPIVLCNCSANVKKLFSAIYNTVNDIAQDIWIFQGWDISTQLREIWYELAISMMKEAEWTKTGFIPSLEEYMENGRTSIALEPIIFTSLFFLGPKLSEQMIHEYKRLMQLLNTCGRLLNDSQSYKREIKQGKLNSLSLFVKEYPATSIEDATEWIRLTVSESRQNILRNLLQPSEIPRECKQIFWNMVKIIQLFYLHSDEFTSPTAMLEHMQAVLFNPVL

SEQ ID NO: 31 - DNA, 2823 bp, Artificial Sequence
  fusion
atgaagatcgaggaaggcaagctcgtgatctggatcaacggcgacaagggctacaacggcctggccgaggtgggcaagaagttcgagaaggacaccggcatcaaggtgacggtggagcacccggacaagctcgaggagaagttcccgcaggtggcggccacgggcgacggcccggacatcatcttctgggcccatgaccgcttcggcggctacgcccagtcgggcctgctggccgagatcaccccggacaaggcgttccaggacaagctctatcccttcacgtgggacgccgtgcgctacaacggcaagctgatcgcgtatcccatcgcggtggaggccctgtcgctcatctataacaaggacctgctcccgaacccgcccaagacctgggaggagatccccgccctcgacaaggagctgaaggccaagggcaagtcggcgctcatgttcaacctgcaggagccgtacttcacctggcccctgatcgcggccgacggcggctacgcgttcaagtatgagaacggcaagtatgacatcaaggacgtgggcgtggacaacgcgggcgccaaggccggcctgaccttcctcgtggacctgatcaagaacaagcacatgaacgccgacacggactactcgatcgcggaggccgcgttcaacaagggcgagactgccatgacgatcaacggcccgtgggcgtggtcgaacatcgacacctcgaaggtgaactatggcgtgaccgtgctccccacgttcaagggccagccctcgaagcccttcgtgggcgtgctgtcggcgggcatcaacgccgcgtcgccgaacaaggagctcgcgaaggagttcctggagaactacctgctcaccgacgagggcctggaggccgtgaacaaggacaagcccctgggcgccgtggccctgaagtcgtatgaggaagagctggtgaaggacccgcgcatcgcggccaccatggagaacgcgcagaagggcgagatcatgccgaacatcccccagatgtcggccttctggtatgcggtgcgcaccgccgtgatcaacgcggcctcgggccgccagaccgtggacgaggccctcaaggacgcccagaccggcggaggtgctgccatgctctcggtggaggactcgtcgaacgtgctcatcaaggccgagaaggagaaccgcgagaagaagtcgttcggctcgatgtgcatcgacgacaacatcacccgcatcaacatgctcttcctcgagatcaagcgccgctactcgtcgctctcggcctacgacaccgtgcacggcgcctacccgatcaacctcatcctcggcctctcgatggtgaaccacctcgagtcgctcggcatcgcccgccacttcaagctcgagatcaaggaggtgctcgacgacgtgtaccgcttctggatcgagaacgagatggtgaagggcatgccgtcggcctcggacatcatgacctcgtcgatctcgttcaagatcctccgctggaacggctacgacgtgtcgccggacgtgttcttctcgtacctccgcgactcggacttcctcccgttcctcgagaactcgggccaggccactatggccctcctcaacctctacaaggcctcgcagatgatgttcccgggcgagcgcatcctcgaggaggccaagtcgttctcgcagaactacctcgagaacatcaaggtgggcgaccagtcgggcgccctcaaggacatcgaggccgaggtgaagcacgccctcgacgtgccgtggatggccggcctcgagcgcatcgagcacctccgctacatgaaggccttcgccttcgacgacatctggatcggcgagacgtcgtcgcgcatcccgttcaccggcaaggacctcttcctctcgctcgccaagcaggacttcaacatctgccaggaggcccagcaggaggacctcaaggccctcgagaagtggtcggtggactcgaagctcggcgacctccacttcgcccgccagaagctcgtggcctgctacttctcggccgcctcgaccctcttctcgccggagatgtcgaccacccgcatcgtgtggaccaagaacgccgtgctcaccaccctcatggacgacttctacgacgtggagggctcgatcgaggacatccagtcgttcgtggaggccgtgaagcgctgggacccgatcgtgctctgcaactgctcggccaacgtgaagaagctcttctcggccatctacaacaccgtgaacgacatcgcccaggacatctggatcttccagggctgggacatctcgacccagctccgcgagatctggtacgagctcgccatctcgatgatgaaggaggccgagtggaccaagaccggcttcatcccgtcgctcgaggagtacatggagaacggccgcacctcgatcgccctcgagccgatcatcttcacctcgctcttcttcctcggcccgaagctctcggagcagatgatccacgagtacaagcgcctcatgcagctcctcaacacctgcggcaggctcctcaacgactcgcagtcgtacaagcgcgagatcaagcagggcaagctcaactcgctctcgctcttcgtgaaggagtacccggccacctcgatcgaggacgccaccgagtggatcaggctcaccgtgtcggagtcgcgccagaacatcctccgcaacctcctccagccgtcggagatcccgcgcgagtgcaagcagatcttctggaacatggtgaagatcatccagctcttctacctccactcggacgagttcacctcgccgaccgccatgctcgagcacatgcaggccgtgctcttcaacccggtgctctaa

SEQ ID NO: 32 - DNA, 2895 bp, Artificial Sequence
  fusion
atgaagatcgaggaaggcaagctcgtgatctggatcaacggcgacaagggctacaacggcctggccgaggtgggcaagaagttcgagaaggacaccggcatcaaggtgacggtggagcacccggacaagctcgaggagaagttcccgcaggtggcggccacgggcgacggcccggacatcatcttctgggcccatgaccgcttcggcggctacgcccagtcgggcctgctggccgagatcaccccggacaaggcgttccaggacaagctctatcccttcacgtgggacgccgtgcgctacaacggcaagctgatcgcgtatcccatcgcggtggaggccctgtcgctcatctataacaaggacctgctcccgaacccgcccaagacctgggaggagatccccgccctcgacaaggagctgaaggccaagggcaagtcggcgctcatgttcaacctgcaggagccgtacttcacctggcccctgatcgcggccgacggcggctacgcgttcaagtatgagaacggcaagtatgacatcaaggacgtgggcgtggacaacgcgggcgccaaggccggcctgaccttcctcgtggacctgatcaagaacaagcacatgaacgccgacacggactactcgatcgcggaggccgcgttcaacaagggcgagactgccatgacgatcaacggcccgtgggcgtggtcgaacatcgacacctcgaaggtgaactatggcgtgaccgtgctccccacgttcaagggccagccctcgaagcccttcgtgggcgtgctgtcggcgggcatcaacgccgcgtcgccgaacaaggagctcgcgaaggagttcctggagaactacctgctcaccgacgagggcctggaggccgtgaacaaggacaagcccctgggcgccgtggccctgaagtcgtatgaggaagagctggtgaaggacccgcgcatcgcggccaccatggagaacgcgcagaagggcgagatcatgccgaacatcccccagatgtcggccttctggtatgcggtgcgcaccgccgtgatcaacgcggcctcgggccgccagaccgtggacgaggccctcaaggacgcccagaccggcggaggtgctgccatggccggcgcctcgaacttcaacttcaacggccgcaagaagttcaacccgcgcacccaccagaaggtgaagtcgctctcggtggaggactcgtcgaacgtgctcatcaaggccgagaaggagaaccgcgagaagaagtcgttcggctcgatgtgcatcgacgacaacatcacccgcatcaacatgctcttcctcgagatcaagcgccgctactcgtcgctctcggcctacgacaccgtgcacggcgcctacccgatcaacctcatcctcggcctctcgatggtgaaccacctcgagtcgctcggcatcgcccgccacttcaagctcgagatcaaggaggtgctcgacgacgtgtaccgcttctggatcgagaacgagatggtgaagggcatgccgtcggcctcggacatcatgacctcgtcgatctcgttcaagatcctccgctggaacggctacgacgtgtcgccggacgtgttcttctcgtacctccgcgactcggacttcctcccgttcctcgagaactcgggccaggccactatggccctcctcaacctctacaaggcctcgcagatgatgttcccgggcgagcgcatcctcgaggaggccaagtcgttctcgcagaactacctcgagaacatcaaggtgggcgaccagtcgggcgccctcaaggacatcgaggccgaggtgaagcacgccctcgacgtgccgtggatggccggcctcgagcgcatcgagcacctccgctacatgaaggccttcgccttcgacgacatctggatcggcgagacgtcgtcgcgcatcccgttcaccggcaaggacctcttcctctcgctcgccaagcaggacttcaacatctgccaggaggcccagcaggaggacctcaaggccctcgagaagtggtcggtggactcgaagctcggcgacctccacttcgcccgccagaagctcgtggcctgctacttctcggccgcctcgaccctcttctcgccggagatgtcgaccacccgcatcgtgtggaccaagaacgccgtgctcaccaccctcatggacgacttctacgacgtggagggctcgatcgaggacatccagtcgttcgtggaggccgtgaagcgctgggacccgatcgtgctctgcaactgctcggccaacgtgaagaagctcttctcggccatctacaacaccgtgaacgacatcgcccaggacatctggatcttccagggctgggacatctcgacccagctccgcgagatctggtacgagctcgccatctcgatgatgaaggaggccgagtggaccaagaccggcttcatcccgtcgctcgaggagtacatggagaacggccgcacctcgatcgccctcgagccgatcatcttcacctcgctcttcttcctcggcccgaagctctcggagcagatgatccacgagtacaagcgcctcatgcagctcctcaacacctgcggcaggctcctcaacgactcgcagtcgtacaagcgcgagatcaagcagggcaagctcaactcgctctcgctcttcgtgaaggagtacccggccacctcgatcgaggacgccaccgagtggatcaggctcaccgtgtcggagtcgcgccagaacatcctccgcaacctcctccagccgtcggagatcccgcgcgagtgcaagcagatcttctggaacatggtgaagatcatccagctcttctacctccactcggacgagttcacctcgccgaccgccatgctcgagcacatgcaggccgtgctcttcaacccggtgctctaa

SEQ ID NO: 33 - DNA, 2895 bp, Artificial Sequence
  fusion
atgaagatcgaggaaggcaagctcgtgatctggatcaacggcgacaagggctacaacggcctggccgaggtgggcaagaagttcgagaaggacaccggcatcaaggtgacggtggagcacccggacaagctcgaggagaagttcccgcaggtggcggccacgggcgacggcccggacatcatcttctgggcccatgaccgcttcggcggctacgcccagtcgggcctgctggccgagatcaccccggacaaggcgttccaggacaagctctatcccttcacgtgggacgccgtgcgctacaacggcaagctgatcgcgtatcccatcgcggtggaggccctgtcgctcatctataacaaggacctgctcccgaacccgcccaagacctgggaggagatccccgccctcgacaaggagctgaaggccaagggcaagtcggcgctcatgttcaacctgcaggagccgtacttcacctggcccctgatcgcggccgacggcggctacgcgttcaagtatgagaacggcaagtatgacatcaaggacgtgggcgtggacaacgcgggcgccaaggccggcctgaccttcctcgtggacctgatcaagaacaagcacatgaacgccgacacggactactcgatcgcggaggccgcgttcaacaagggcgagactgccatgacgatcaacggcccgtgggcgtggtcgaacatcgacacctcgaaggtgaactatggcgtgaccgtgctccccacgttcaagggccagccctcgaagcccttcgtgggcgtgctgtcggcgggcatcaacgccgcgtcgccgaacaaggagctcgcgaaggagttcctggagaactacctgctcaccgacgagggcctggaggccgtgaacaaggacaagcccctgggcgccgtggccctgaagtcgtatgaggaagagctggtgaaggacccgcgcatcgcggccaccatggagaacgcgcagaagggcgagatcatgccgaacatcccccagatgtcggccttctggtatgcggtgcgcaccgccgtgatcaacgcggcctcgggccgccagaccgtggacgaggccctcaaggacgcccagaccggcggaggtgctgccatggccggcgcctcgaacttcaacttcaacggccgcaagaagttcaacccgcgcacccaccagaaggtgaagtcgctctcgcgccgcgactcgtcgaacgtgctcatcaagtgggagaaggagaaccgcgagaagaagtcgttcggctcgatgtgcatcgacgacaacatcacccgcatcaacatgctcttcctcgagatcaagcgccgctactcgtcgctctcggcctacgacaccgtgcacggcgcctacccgatcaacctcatcctcggcctctcgatggtgaaccacctcgagtcgctcggcatcgcccgccacttcaagctcgagatcaaggaggtgctcgacgacgtgtaccgcttctggatcgagaacgagatggtgaagggcatgccgtcggcctcggacatcatgacctcgtcgatctcgttcaagatcctccgctggaacggctacgacgtgtcgccggacgtgttcttctcgtacctccgcgactcggacttcctcccgttcctcgagaactcgggccaggccactatggccctcctcaacctctacaaggcctcgcagatgatgttcccgggcgagcgcatcctcgaggaggccaagtcgttctcgcagaactacctcgagaacatcaaggtgggcgaccagtcgggcgccctcaaggacatcgaggccgaggtgaagcacgccctcgacgtgccgtggatggccggcctcgagcgcatcgagcacctccgctacatgaaggccttcgccttcgacgacatctggatcggcgagacgtcgtcgcgcatcccgttcaccggcaaggacctcttcctctcgctcgccaagcaggacttcaacatctgccaggaggcccagcaggaggacctcaaggccctcgagaagtggtcggtggactcgaagctcggcgacctccacttcgcccgccagaagctcgtggcctgctacttctcggccgcctcgaccctcttctcgccggagatgtcgaccacccgcatcgtgtggaccaagaacgccgtgctcaccaccctcatggacgacttctacgacgtggagggctcgatcgaggacatccagtcgttcgtggaggccgtgaagcgctgggacccgatcgtgctctgcaactgctcggccaacgtgaagaagctcttctcggccatctacaacaccgtgaacgacatcgcccaggacatctggatcttccagggctgggacatctcgacccagctccgcgagatctggtacgagctcgccatctcgatgatgaaggaggccgagtggaccaagaccggcttcatcccgtcgctcgaggagtacatggagaacggccgcacctcgatcgccctcgagccgatcatcttcacctcgctcttcttcctcggcccgaagctctcggagcagatgatccacgagtacaagcgcctcatgcagctcctcaacacctgcggcaggctcctcaacgactcgcagtcgtacaagcgcgagatcaagcagggcaagctcaactcgctctcgctcttcgtgaaggagtacccggccacctcgatcgaggacgccaccgagtggatcaggctcaccgtgtcggagtcgcgccagaacatcctccgcaacctcctccagccgtcggagatcccgcgcgagtgcaagcagatcttctggaacatggtgaagatcatccagctcttctacctccactcggacgagttcacctcgccgaccgccatgctcgagcacatgcaggccgtgctcttcaacccggtgctctaa

SEQ ID NO: 34 - protein, 631 aa, Cupressus gigantea
MAALSFSTISTTANYAIPKLPSSNLQGRSRDTKYHVLIRSAGASNFNFNGRKKFNPRTHQKVKSLSVEDSSNVLIKAEKENREKKSFGSMCIDDNITRINMLFLEIKRRYSSLSAYDTVHGAYPINLILGLSMVNHLESLGIARHFKLEIKEVLDDVYRFWIENEMVKGMPSASDIMTSSISFKILRWNGYDVSPDVFFSYLRDSDFLPFLENSGQATMALLNLYKASQMMFPGERILEEAKSFSQNYLENIKVGDQSGALKDIEAEVKHALDVPWMAGLERIEHLRYMKAFAFDDIWIGETSSRIPFTGKDLFLSLAKQDFNICQEAQQEDLKALEKWSVDSKLGDLHFARQKLVACYFSAASTLFSPEMSTTRIVWTKNAVLTTLMDDFYDVEGSIEDIQSFVEAVKRWDPIVLCNCSANVKKLFSAIYNTVNDIAQDIWIFQGWDISTQLREIWYELAISMMKEAEWTKTGFIPSLEEYMENGRTSIALEPIIFTSLFFLGPKLSEQMIHEYKRLMQLLNTCGRLLNDSQSYKREIKQGKLTSLSLFVKEYPAHLIEDATEWIRLTVSESRQNILRNLLQPSEIPRECKQIFWNMVKIIQLFYLHSDEFTSPTAMLEHMQAVLFNPVL

SEQ ID NO: 35 - DNA, 1896 bp, Cupressus gigantea
atggcagcacttagtttttctaccatctccaccactgccaactatgcaattcctaagctgccatcttcaaatcttcaagggcggtccagagatacaaaataccatgtcttgataagatctgcaggggcctctaatttcaacttcaatggaagaaagaagttcaatccaagaacacatcagaaagtcaaatctttgagtgttgaagattcctccaatgtacttattaaggctgagaaagagaacagagagaaaaaatcatttggttcgatgtgcatagacgataatattacaaggataaatatgttgttcttggaaattaagagaagatattcatctctttctgcttatgatacagtgcacggtgcttatcctatcaatttgattcttggcctctcaatggtaaatcatttggagagcttgggtatagcaagacatttcaaacttgagataaaagaggttctagatgatgtctatagattttggatagagaatgaaatggttaaaggaatgccatcagcatcagacataatgaccagttctatcagtttcaagatcttgcggtggaatggatatgacgtgtctccagatgtctttttcagttacctgagggatagtgacttcctaccatttctagaaaatagtggccaagctactatggcattgctgaatttatacaaggcatctcagatgatgttcccaggggaaagaattcttgaagaagcaaaatcgttctcccaaaattatcttgaaaatatcaaggtgggcgatcaaagtggagctctcaaagatattgaagcagaggttaagcatgcactggatgttccttggatggctggtttggagagaatagaacatctgcgatacatgaaagcatttgcttttgatgatatttggattggagagacctcttctaggatcccatttacaggaaaggatttatttttatctttagccaaacaagacttcaatatctgccaagaagctcaacaagaggatttgaaagcgttggaaaaatggagtgtagactctaaacttggtgacctacactttgctagacaaaagcttgtagcctgctatttctctgccgcctcaacactttttagccctgaaatgtctactactcggattgtgtggactaaaaatgctgttctcactactctaatggatgacttttatgatgtggagggttccattgaagatattcaatcctttgttgaagcagttaagagatgggacccaatagttttatgtaattgttcagctaatgtgaaaaaattattctctgctatttataacactgtgaatgatattgctcaagatatatggattttccagggctgggacattagcactcagttaagagaaatttggtacgaactggcaatctcaatgatgaaggaggctgaatggaccaaaactggatttataccatcgctggaagaatacatggaaaatgggagaacgtcaattgccttggaaccaattatttttacatcactattttttcttggccctaagctttccgagcaaatgatccacgagtataagagacttatgcaactcctgaatacttgtgggcgccttttgaatgacagtcagagctacaagagggagataaagcaaggaaagctcacctctttatcattgttcgtgaaagaatatcccgcacatcttattgaggatgctactgagtggattagactcactgtaagtgaaagcagacaaaatatactcagaaatttattgcaaccaagtgagatccctcgtgaatgcaagcagatattttggaatatggtcaaaattatccaactattctacctccacagtgatgaattcacatctcctactgcaatgcttgaacatatgcaagcagttctttttaatcctgttttataa

SEQ ID NO: 36 - DNA, 1650 bp, Artificial Sequence
  truncated
atggagaaaaaatcatttggttcgatgtgcatagatgatgatattacaaagataaaaatgttgttctcggaaattaaaagaagatattcatctctttctgcttatgatacagtgcacggtgcttatcctatcaatttgattcttggcctctcaatggtaaaccacttggagagcttgggtatagcaagacatttcaaacttgagataaaagaggttctagatgatgtctatagattttggatagagaatgaaatggttgaaggaatgccatcagcatcagacataatgaccagttctatcagtttcaggatcttgcggtggaatggatatgacgtgtctccagatgtctttttcagttacctgagggatagtgacttcctactatttttagaaaatagtgaccaagctactatagcattgctgaatttatacaaggcatctcagatgatgttcccaggcgaaagaattcttgaagaagcaaaattgttctcccaaaattatcttgaaaatatcaagatgggcaatcaaagtggagctctcaaagacattgaagcagaggttaagcatgcactggatgtcccttggatggctaatttggagagaatagaacatctgcgatacatgaaagcatttgcttttgacaatatttggattggagagacctcttctaggatcccatttacaggaaaggatttatttttatcttttgccaaacaagacttcaatatctgccaagcagctcaacaagaggatttgaaggcgttggaaaaatggagtgtagactctaaacttggtgacctacactttgctagacaaaagcttgtagcctgctatttctctgccgcctcaatactttttagccctgaaatgtctgctactcggattgtgtggactaaaaatgctgttctcattacactaatggatgacttttatgatgtggagggttccatygaagatattcaatcctttgttgaagcggttaagagatgggacccaatagttttatgtaattgttcagctaatgttaaaattttattctctgctatttataacactgtgaatgatattgctcaagatatatggattttccagggctgggacattagcactcagttaagagaaatttggtacgaactggcaatctcaatgatgaaggaggctgaatggaccaaaactggatttataccatccttggaagaatacatggaaaatgggagaacatcaattgccttggaaccaattatttttacatcactattttttattggccctaagctttccgagcaaatgatccaccatcacgagtataagaggcttttgcaactcatgaatacttgtgggcgccttttgaatgacattcagagcgacaagagggagatgaagcaaggaaagctcacctctgtatcattgttcgtgaaagaatacccaattgaggatgctactaagtggattagactcactgtaagtgaaaacacacaaaatatactcaaaaaaatattgcaaccaagtgagatccctcgtgaatgcaagcagatattttggaatatggtcaaaattatccaactattctgcctccacagtgatgaattcacatctcctaccacaatgcttgaacatatgaaggcagttcttttttatcctgttttataa

SEQ ID NO: 37 - DNA, 1653 bp, Artificial Sequence
  truncated
atggagaaaaaatcatttggttcgatgtgcatagacgataatattacaaggataaatatgttgttcttggaaattaagagaagatattcatctctttctgcttatgatacagtgcacggtgcttatcctatcaatttgattcttggcctctcaatggtaaatcatttggagagcttgggtatagcaagacatttcaaacttgagataaaagaggttctagatgatgtctatagattttggatagagaatgaaatggttaaaggaatgccatcagcatcagacataatgaccagttctatcagtttcaagatcttgcggtggaatggatatgacgtgtctccagatgtctttttcagttacctgagggatagtgacttcctaccatttctagaaaatagtggccaagctactatggcattgctgaatttatacaaggcatctcagatgatgttcccaggggaaagaattcttgaagaagcaaaatcgttctcccaaaattatcttgaaaatatcaaggtgggcgatcaaagtggagctctcaaagatattgaagcagaggttaagcatgcactggatgttccttggatggctggtttggagagaatagaacatctgcgatacatgaaagcatttgcttttgatgatatttggattggagagacctcttctaggatcccatttacaggaaaggatttatttttatctttagccaaacaagacttcaatatctgccaagaagctcaacaagaggatttgaaagcgttggaaaaatggagtgtagactctaaacttggtgacctacactttgctagacaaaagcttgtagcctgctatttctctgccgcctcaacactttttagccctgaaatgtctactactcggattgtgtggactaaaaatgctgttctcactactctaatggatgacttttatgatgtggagggttccattgaagatattcaatcctttgttgaagcagttaagagatgggacccaatagttttatgtaattgttcagctaatgtgaaaaaattattctctgctatttataacactgtgaatgatattgctcaagatatatggattttccagggctgggacattagcactcagttaagagaaatttggtacgaactggcaatctcaatgatgaaggaggctgaatggaccaaaactggatttataccatcgctggaagaatacatggaaaatgggagaacgtcaattgccttggaaccaattatttttacatcactattttttcttggccctaagctttccgagcaaatgatccacgagtataagagacttatgcaactcctgaatacttgtgggcgccttttgaatgacagtcagagctacaagagggagataaagcaaggaaagctcaactctttatcattgttcgtgaaagaatatcccgcaacatctattgaggatgctactgagtggattagactcactgtaagtgaaagcagacaaaatatactcagaaatttattgcaaccaagtgagatccctcgtgaatgcaagcagatattttggaatatggtcaaaattatccaactattctacctccacagtgatgaattcacatctcctactgcaatgcttgaacatatgcaagcagttctttttaatcctgttttataa

SEQ ID NO: 38 - DNA, 1653 bp, Artificial Sequence
  truncated
atggagaaaaaatcatttggttcgatgtgcatagacgataatattacaaggataaatatgttgttcttggaaattaagagaagatattcatctctttctgcttatgatacagtgcacggtgcttatcctatcaatttgattcttggcctctcaatggtaaatcatttggagagcttgggtatagcaagacatttcaaacttgagataaaagaggttctagatgatgtctatagattttggatagagaatgaaatggttaaaggaatgccatcagcatcagacataatgaccagttctatcagtttcaagatcttgcggtggaatggatatgacgtgtctccagatgtctttttcagttacctgagggatagtgacttcctaccatttctagaaaatagtggccaagctactatggcattgctgaatttatacaaggcatctcagatgatgttcccaggggaaagaattcttgaagaagcaaaatcgttctcccaaaattatcttgaaaatatcaaggtgggcgatcaaagtggagctctcaaagatattgaagcagaggttaagcatgcactggatgttccttggatggctggtttggagagaatagaacatctgcgatacatgaaagcatttgcttttgatgatatttggattggagagacctcttctaggatcccatttacaggaaaggatttatttttatctttagccaaacaagacttcaatatctgccaagaagctcaacaagaggatttgaaagcgttggaaaaatggagtgtagactctaaacttggtgacctacactttgctagacaaaagcttgtagcctgctatttctctgccgcctcaacactttttagccctgaaatgtctactactcggattgtgtggactaaaaatgctgttctcactactctaatggatgacttttatgatgtggagggttccattgaagatattcaatcctttgttgaagcagttaagagatgggacccaatagttttatgtaattgttcagctaatgtgaaaaaattattctctgctatttataacactgtgaatgatattgctcaagatatatggattttccagggctgggacattagcactcagttaagagaaatttggtacgaactggcaatctcaatgatgaaggaggctgaatggaccaaaactggatttataccatcgctggaagaatacatggaaaatgggagaacgtcaattgccttggaaccaattatttttacatcactattttttcttggccctaagctttccgagcaaatgatccacgagtataagagacttatgcaactcctgaatacttgtgggcgccttttgaatgacagtcagagctacaagagggagataaagcaaggaaagctcacctctttatcattgttcgtgaaagaatatcccgcacatcttattgaggatgctactgagtggattagactcactgtaagtgaaagcagacaaaatatactcagaaatttattgcaaccaagtgagatccctcgtgaatgcaagcagatattttggaatatggtcaaaattatccaactattctacctccacagtgatgaattcacatctcctactgcaatgcttgaacatatgcaagcagttctttttaatcctgttttataa

SEQ ID NO: 39 - DNA, 1550 bp, Artificial Sequence
  truncated
atggagaagaagtcgttcggctcgatgtgcatcgacgacgacatcaccaagatcaagatgctcttctcggagatcaagcgccgctactcgtcgctctcggcctacgacaccgtgcacggcgcctacccgatcaacctcatcctcggcctctcgatggtgaaccacctcgagtcgctcggcatcgcccgccacttcaagctcgagatcaaggaggtgctcgacgacgtgtaccgcttctggatcgagaacgagatggtggagggcatgccgtcggcctcggacatcatgacctcgtcgatctcgttccgcatcctccgctggaacggctacgacgtgtcgccggacgtgttcttctcgtacctccgcgactcggacttcctcctcttcctcgagaactcggaccaggccaccatcgccctcctcaacctctacaaggcctcgcagatgatgttcccgggcgagcgcatcctcgaggaggccaagctcttctcgcagaactacctcgagaacatcaagatgggcaaccagtcgggcgccctcaaggacatcgaggccgaggtgaagcacgccctcgacgtgccgtggatggccaacctcgagcgcatcgagcacctccgctacatgaaggccttcgccttcgacaacatctggatcggcgagacgtcgtcgcgcatcccgttcaccggcaaggacctcttcctctcgttcgccaagcaggacttcaacatctgccaggccgcccagcaggaggacctcaaggccctcgagaagtggtcggtggactcgaagctcggcgacctccacttcgcccgccagaagctcgtggcctgctacttctcggccgcctcgatcctcttctcgccggagatgtcggccacccgcatcgtgtggaccaagaacgccgtgctcatcaccctcatggacgacttctacgacgtggagggctcgatcgaggacatccagtcgttcgtggaggccgtgaagcgctgggacccgatcgtgctctgcaactgctcggccaacgtgaagatcctcttctcggccatctacaacaccgtgaacgacatcgcccaggacatctggatcttccagggctgggacatctcgacccagctccgcgagatctggtacgagctcgccatctcgatgatgaaggaggccgagtggaccaagaccggcttcatcccgtcgctcgaggagtacatggagaacggccgcacctcgatcgccctcgagccgatcatcttcacctcgctcttcttcatcggcccgaagctctcggagcagatgatccaccaccacgagtacaagcgcctcctccagctcatgaacacctgcggccgcctcctcaacgacatccagtcggacaagcgcgagatgaagcagggcaagctcacctcggtgtcgctcttcgtgaaggagtacccgatcgaggacgccaccaagtggatccgcctcaccgtgtcggagaacacccagaacatcctcaagaagatcctccagccgtcggagatcccgcgcgagtgcaagcagatcttctggaacatggt

SEQ ID NO: 40 - protein, 516 aa, Artificial Sequence
  truncated
MEKKSFGSMCIDDDITKIKMLFSEIKRRYSSLSAYDTVHGAYPINLILGLSMVNHLESLGIARHFKLEIKEVLDDVYRFWIENEMVEGMPSASDIMTSSISFRILRWNGYDVSPDVFFSYLRDSDFLLFLENSDQATIALLNLYKASQMMFPGERILEEAKLFSQNYLENIKMGNQSGALKDIEAEVKHALDVPWMANLERIEHLRYMKAFAFDNIWIGETSSRIPFTGKDLFLSFAKQDFNICQAAQQEDLKALEKWSVDSKLGDLHFARQKLVACYFSAASILFSPEMSATRIVWTKNAVLITLMDDFYDVEGSIEDIQSFVEAVKRWDPIVLCNCSANVKILFSAIYNTVNDIAQDIWIFQGWDISTQLREIWYELAISMMKEAEWTKTGFIPSLEEYMENGRTSIALEPIIFTSLFFIGPKLSEQMIHHHEYKRLLQLMNTCGRLLNDIQSDKREMKQGKLTSVSLFVKEYPIEDATKWIRLTVSENTQNILKKILQPSEIPRECKQIFWNM

SEQ ID NO: 41 - protein, 631 aa, Artificial Sequence
  variant 1
MAALSFSTISTTANYAIPRIPSSNLQGRSRDTKYHVLIRSAGGSNFNFNGRKKFNPRTHQKIKSLSVEDSSNVLIKAEKEQREKKSFGSMCIDDNITRIQMLFLEIKRRYSSLSAYDTVHGAYPINLIIGLSMVNHLESLGIGRHFKLEIKEVLDDVYRFWIENEMVKGMPSASDIMTSSISFKILRWNGYDVSPDVFFSYLKDSDFLPFLENSGQATMALLNLYKASQMMFPGERILEEAKSFSQNYLENIKVGDQSGALKDIEADVKHALDVPWMAALERIEHLRYMKAFAFDDIWLGETSSRIPWTGKDLFLSLAKQDFNICQEAQQEDLRALEKWSVDSKLGELHFARQKLVACYFSAASSLFSPEMSTTRIVWTKNAVLTTLMDDFYDVEGSLEDIQSFVEAVRRWDPIVLCNCSAQVKKLFSAIYNTVNDIAQDVWIFQGFDISTQLREIWFELAISMMKEAEWTKTGFIPTLEEYMENGRTSIALEPIIFTSLFFLGPKLSENMIHEYKRLMQLLNTCGRLLNDSQSYKREVKQGKLNSLSLFVKEYPASSIEDATEWIRLTVTESRQNILRNLLQPSEIPRECKQIFWNKVKLIQLFYLHSDEFTSPTAMLEHMQAVVYNPLL

SEQ ID NO: 42 - protein, 631 aa, Artificial Sequence
  variant 2
MGGLSFSTISTTANWALPKLPSSNLQGMSRDTKYHVLIRSAAASNFNFNGRRKFNPRTHQKIKSLSVEDTSNVLIKAEKENRDKKSFGSMCIDDNITRLNMLFLEIKKKYSSLSAFDTVHGAYPINVILGLSMVNHLESLGIAMHFMLEIKEILDDVYRFWIENEMLKGKPSASDIMTSSISFKILRWNGYDVSPDVFFSYLMDSDFLPYVENSAQASMALLNLYKASQMMFPGERILEEAKSFSQNYLDNIKIGDQSAALKDIEAEVKHALDVPWMAGLERIEHLRYMKAFAFDDIYIGETSSMIPFTGRDLFLSLAKQDFNICQEAQQEDLRALEKWSVDSKLGDLHFARQKLVGCYFTAASTLFSPDMSTTRILFTKNAVLTTLMDDFYDVEGSIEDIQSFVEAVKRWDPIVLCNCSAQVKKLFSAIYNTINDIAQDVYIFQGWDISTQLREIYYELAVSMMKEAEWTKTAYIPSLEEYMENGRTSIALEPIIFTSLFWLGPKLSEQMIHEFKRLMQLLNTCGRLLNDSQSWKREIKNGKLQSLTLFVKDYPATSIEDATDYIRVTVSESRQNILKNLLQPSELPRECRQIFWNMVKIINLFYLHSDDFTSPTAMLEHMQGLLYNPVI

SEQ ID NO: 43 - protein, 631 aa, Artificial Sequence
  variant 3
MAALSFTTISTTANYAIPKLPSTNVNGRSRDTRYHVLIRSAAGSNFQFNGRKKFNPRTHQKVKSLSVEDSSNVLIKAEKENREKKSFGSMCVDDNLTRIQMLFLEIMRRWSSLSAYDTVHGAWPLNLIVGLSRVNHLESLGIARHFKLEIKEVLDDLYRYYIENEMVKGMPSASEIMTSSISFKILRWNGYDVSPDVFFSYLRDTDFLPFLDNTGQATMALLNLYKASQMRFPGERILDEAMSFSQNYLENIKVGDQSGALKDIEAEVRHALDVPWMAGLERIEHLRYMKGFGYDDIYIGETSSRIPWTGKDIFLSLAKQDFNLCQEAQQEDLKALDMWSVDSKLGDLHFARQMLVGCYFSAASTLFSPEMSSTRIVFTKNAVLTTLKDDFYDVEGSLEDIQSFVEAIKRWDPIVVCNCSANVKRLFSAIYNSVNDIGQDIWIFQGWDISTQLREIWYEVAISMMKEAEWTMTGFLPTVEEYMENGRTSLALEPIIFTSLFYLGPKISEQRIHEYKRLRQVLNTCGRLLNDSQSYKMEIKQGKLNSLSLFVKEYPATSIEDASEWIRLTVSESRQNILRNILQPSELPRECKQIFWNMVKIIQLFYLHSDEFTSPTAMLEHMQAVLWNPVL

SEQ ID NO: 44 - protein, 631 aa, Artificial Sequence
  variant 4
MAALSFSTISTTANYAIPKIPTSNLQGRSRDTMYHVLIRSAGATNYNFQGRRKFQPRTHQKVKSLSVEDSSNVVIKAENENREKKSFGSMCIDDNVTRLNMLFLEIKRRYSSLTGYDTVHGAYPINLILGLSMVNHLESLGIARHFMLEIREVLDDVFRFWIENEMVKGKPSASDIMSTSISFKILRWNGYDVSPDVFFSYLRESDFLPFLENSGQASMALLNLYKASQMMFPGEMILEEARSFSQNWLEQIKVGDQSGALKDIEAEVMHALDVPFMAGLERIEHLRYMKAFAFDDVWIGETSSRIPFTGKELFLSLAKQDFNICQEAQQEDLKALEKWSVDTRIGDLHFARQMLLGCYFSAASTLYSPEMSTTRIVWTKNAVLTTLKDDFYDVDGSIEDIQSFVEAIKRWDPLVLCNCSANVKKLFTAIYNTVNDVAQDIWIFQGWDISTQLREIWYEVAITRMKEAEWTMTGYVPSLEEYMENGRTSIALEPIIFTSLFFLGPKVSEQMIHEYKRLKQLLNTCGRLLNDTQTYKREIMQGKLQTLSLFVREYPATSIEDASEWIRLTLSESRQNILRQLLQPSEIPRECKQIYWNMIKVIQLFYLHSDEFTSPTAMLEHMQAVLFNPVL

SEQ ID NO: 45 - protein, 631 aa, Artificial Sequence
  variant 5
MAALSFSTISTTGNYAIPKLPSSNLQGRSRETKYHVLVRSGGASNFNFNARKMFNPRTHQKVMSLSVEESSNVLIKAEKEQRENKSFGSMCIEDNITMINMLFVEIRRRYSSVTAYDTVHGAYPINLLLGLSMVNHLESLGIGKHFKLEIKEVLDDVWRFWIDNDMLMGMPSASDIKTSSISFKILRWNGYDVSPDLFFTYIRDSDFLPFVEQSGQATMALLNLYKASQKMFPGERLLEEAKSFSQNYLENIMLGDQSGGLKDVDGEVKHALDVPWMAGIERVEHLRFMMAFAFDDIWIGETSSRIPFTGKDLFLSLAKNDFNICQEAQNEDLKGLEKWSVDSRLGDLHFARNKIIACYFSGATTLFSPEMSTSRIVWTKNAVLTTLRDDFYDVDGSIEDIQSFIEAVKRWDPIVLCNCSANVKKLFSAIYNTVNDVAQEIWIYQAFEVSTQLREIWFEVAISMMKEAEWTKSGFIPSLEEYMENGRTSIGLEPIIFSSIFFLGPRITDQMIHEYKRLMQLVNTCGRLLNDSQTFKREVMQGKVNSLSLFVREYPASSIEDATEWVRLTVSESRQNLLRNILNPSEVPRECKQLFWNMVKILQLFYLHTDDFTSPTAMLEHMNAVLWQPVI

SEQ ID NO: 46 - protein, 631 aa, Artificial Sequence
  variant 6
MAAVSWSTISTTAQYALPKIPSSNLNARSMDTRYHVLIRSAGASNWNFNARKKWQPRTHQKVKSLSVEDSTNLLIKAERENREKKSFGSMCIDDNITRIQMLWLEIMRRYSSISAYDSVHAAYPLNIILGLSMVNHLDSLGIARHFKLEIKEVLDDLYMFFIEQEMVRGMPSATDIMSSSISFKILRWQGYEVSPDVFFSYLRDTDFLPWIDNSGNATMALLNLYKASQMMFPGEKILEEAKSFSQNYLENIKVGDQSGGLMDIEAEVKHALDLPWMGGLERVEHLRYMMGFAFEDLWIGETSSKIPFTGKDLFLSLAKNDFNICQEAQQEDLKALEKWSLDSKLGELHFARQKLVACYWSAATTLYSPEMSSTRLVWTKNAVLTTIMDDFYDVDGSIEDIQSFVEAVKRWDPIVLCQCSANVKKLFSAIFNTVNDIGQDIWIFQAWDISTQLRELWFDLAISMMKEAEWTKTGFVPSVEEYMENGRTSLAIEPIIFTSLYFLGPKLSEQMVHEYKMLKQLVNTCGRLINDTQSYKMEVKQGRVNSVSLFVKEYPASSIEDATEWIRLTVSETRQNLLKNLVQPSEIPKECKQLFWNMVKIINLFYVHSDEFTSPTAKLEHMQAILFNPVL

SEQ ID NO: 47 - protein, 631 aa, Artificial Sequence
  variant 7
MGGLSWSTITTTANWAIPKLPSSNVQGRSRDTMYHVLIKSGGATNFNFNGRKRFQPRTHQKIRSLSVDDSSNVLIRAEKDNREKKSFGSMCIDDNITRINKLYLDIKMRYSSISAYDTVHGAYPINLILGLSMVNHLESLGIARHFKLEIREVLDDLYRFWIENEMVKGMPSASDIMTSSITFKILRWNGYDVSPDVFFSYLRDSDFVPFLENSAQASMALLQLYKASQRMFPGERVLEEAMSFSQNYLENLMVGEQSGAVMEIEAEVKHALDLPWMAGLERIDHLKWMRAFAFDDIWLGESSSRVPFTGKDLFLSLAKQDFNICQEAQQEDLMGVEKWSVDSKIADLHFARQKIVACYYSAASTLFSPEMSSTRIVWTKNALLSTLMDDFYDLEGSIEDIQTFIEAIKRWDPILLCNCSANIKKLFSAIYQTVNDIANDIWIWQGWDISTQLRELWYEVGISMMKEAEWTKTGYIPSLEEYMENGRTSIALEPIIFTSLFWLGPRLSEQMIHEYKKLMQLLNTCGRLLNDSQTYKREVKQGKLNSVTLFVKEYPATSVEEASEWIMLTLTESRQQILRNILQPSEIPRECKQIFWNRVKIIQLFYLHSDEWSSPTAMLEHMNAIIWQPVL

SEQ ID NO: 48 - protein, 631 aa, Artificial Sequence
  variant 8
MGAVTFSSISTTANYGIPMIPSSNLQGRSRDTRFHVLIMSAAASQFNWQGKMMFNPKTHQRVMSLSVEDSSNVLVMAEKENRENKSFGSMCIDENLTRINMLFLDIMRMYSSVSGFDTVHGAYPVNLIVGLSMVNHLESLGIAMHFKVEIRELLDDVWMFWIEQEMIRGMPTGSDIMTSSISFKILRWNGYDVSPDVFFTYVRDSEFLPFIENSGQATMALLNLYKASQMKYPGERILEEAKSFSQNYLDNIKVGDQSGAIRDLEGDVKHALDVPWMGGLDRLEHLKYMRAFAFDDIWIGETSSRIPFTGRDLFLSLAKQDFNVCQEAQQEDLRALEKWSVDSKLGDLHFARNMLVACYWSGASSIWSPEMSTTRVVWTKNAIITTLMDDFYDIEGSIEDIQSFVEAVKRWDPLVLCNCSANVKMLFSAIYNTVNDIAQEIWIYNGWDLTTQLMEIYFDLGISMRKEAEWTMTAFIPSLDEYKDNGRTSIALEPIIFTSIFFLGPRVSEQRLHEYKRLMQLLNTCARLLNDSQSWMRELRQGMLNSLSLFVKEWPGTSIEDATEYIRLTLSDTRQNILMQLLQPTEVPRDCRQIFWNMVKVVQVYYLHSDDFTTPTAMLEHMQAVLWNPLI

SEQ ID NO: 49 - protein, 631 aa, Artificial Sequence
  variant 9
MAAVSFSTITSTANYAIPMLPTSQVQGKSRDTRFHVLIKTAGASQFNYQARKKFQPRTHQKVKSLSLEDSSNVLVMADREQREKKSFGSMCIDENITRVNMLFLEIKRMWSSLSAFETVHGAWPINIILGLSKVNHLESLGIAKHFKIEIMELLDEVWRYWIDNDKLMGMPSASDIMSSSITFKILRWNGYDVSPDVFFSYVMDSEFIPFLEQSGQATMALLNLYKASQMMFPGERILEEAMSFSQQYLENLRVADQSAAIKDLEGEVRHALDVPYRGGLERLEHLRWMKAFAFDDIWIGETSSRIPFTGKDIFLSLAKQDFNICQEAQNEDLRAIEKWSVDSKLGDLHFARQRLVGCYYSAASTIFSPEMSTTRIVFTKNGVLSTLMDDYYDVEGSIDDIQSFVEAVMRWDPIVLCNCSGNIKKLFSAIYNTVNEIANDVWIYNGWDISTNLKEIFYEVAISKMKEAEWTKTGWIPSLDEYMENGRTSIALEPIIFTSLFFLAPKLTEQMIHEWKRLMNLINTCGRLLNDSQSWKREIKQGKLQTLSLFVMDFPATSLEDATEWLMLTVSESRQNLLMNLLQPSELPKECKNIFFNMIKILQLFYLHSDEWTSPTARVEHMNGVLFNPLV

SEQ ID NO: 50 - protein, 631 aa, Artificial Sequence
  variant 10
MGGLTFTTISTTGNYGIPKLPSSNIQGKSMDTMYHLLIRSAGATQWNYQARRKFNPRTHQMVRSLSIEDSSNVLIKAEMENRENKSFGSMCIDDNITKLNRLWVEIRRRYSSLSGWDTVHAAYPLNLILGLSMVNHLESLGIAKHFKVEIKEVLDDVWKYWIEQERLRARPTATDIMTTSISFKILRWNGYDVSPDVFFSYLRESDFVPFLEQSGNATMAVLQLYKASQMMFPGERVLEEAKSFSQNYLENIMVGEQSGGIMDVEGDVKHGLDLPWMAGLERIEHLMYMKAFAFDDIWVGETSTRLPFTGRDLFLSLAKQDFNICQEAQQEDLMALEKWSVDSKLGDLHFARQKLIACYWSAASTVYSPEMSTSRLVWTKQAVITTIMDDWYDIEGSIDDIQSFVDAVKRWDPIVLCNCSANLKMLFSAIYNTVNELAQDIWIYQGWDISTQLMDIWYEIGISMKMEAEWTKTAWVPSLEEYKENGRTSIALEPIIFTTIFFVGPMLSEQRIHDYKRLMQLLNTCGRLLNDSQSWKREVRQGKLNTLSLFVKEYPGTSIEDATEYIRVTISESRQNVLMNLINPSEVPRDCKQIFFNRIKIIQLFYLHSDEFTSPTAKLDHMQAIVYNPIL

SEQ ID NO: 51 - protein, 631 aa, Artificial Sequence
  variant 11
MGGLTFTTVSTTANYAVPKLPSSNIQGKSRETMFHILLRTAAATQFNWQARMKFQPKTHNKVMTLSVEDSTNIVLKAEKENRENKSFGSMCIDDNLTRVNMLYVDIKMRWSSVSAYETVHGAYPLNVLLGLTRVNHLESLGIAKHFRVEIREILDDVYRWYLDNDRVKAKPSASDIMTTSISFKILRYNGYDVSPDVFYSYLRDSEFLPFIDNSGNASMAVLQLYKASQMMFPGERVLEEAKSFSQNYLDNIKVADQTAALRDVDAEVMHGLDVPYMAGVDRIEHLRWMKAFGYEDIYVGETSSMIPFTAMDLFLSLAKQDFNICQEGQQEDLKALEMWSLDSKLGELHFARNMLLACYFSAGSTLWSPEMSTTRIVWTKNAVLTTLKDDYYDIEGSVEDINSFVEALRRWDPLIVCNCSGNIKKLFSAIYNSLNELAQEIWLFQGWEISTNIMEIWYELAISMMKEAEWTMTAFIPTLDEYMENGRSSIALEPIIFTSIWYLGPKLSEQKIHEYMRLMQVVNTCARLLNDSQTYMRELKNGKLNSLSLFVREYPGTSIEDATEWIRITLTETRQQILRNVIQPSEIPRDCKQIFWNRLKIVQLFYLHSDEFTSPTAMVEHMQAILFNPVI

SEQ ID NO: 52 - protein, 631 aa, Artificial Sequence
  variant 12
MAGISYTTLSTTANYGVPRLPSSNIQGRTKDSKWHVLIRTGAGSQYNYNGRKKYQPRSHNKVKSLSVEETSNILIKADKDNREKKSFGSMCIDENISKINMLYLEIKRKYSSITAFETVHGAWPLNLILGLSKVNHLESLGIARHFRLEIKEVLDDVYRFYIENEKVKGKPSASDIMTSSISFKILRWNGYDVSPDVFWSYVRDTDFLPFVENTGQATMAVLNLYKASQRMYPGEKVLEEARSFSQNYLENVKVGDNSGAIKDIDAEVRHALDVPWRAALERVEHLRYMKAFGFEELWLGETSSKIPFTAKDLFLTLAKNDFNICQEAQQDDLMAVERWSLDSKIADLHFARQRVIACYFSAGSTLFSPEKSTTRLVYTKNAVLTTLMDDWYDVEGSLEDIQSFVDAVKRWDPIVVCNCSANLKMVFSAIFNTVNDLAQEVFVFNAWDLSTNLRDLWYDVAISMMREAEWTKTGWLPTIEEYMENGKTSIALEPIIFTTVFFLGPMITEQMVHDWRRLKQLLNTCGRLINDSNSFKMELKNGKVNSVSLFVKEWPATSIEDASEYIKVTLSESRQNILKQLVQPSEIPKECKQIFWNKVKLLQVFYLHSDDFTTPTGMLEHMQGVVFNPVV

SEQ ID NO: 53 - protein, 631 aa, Artificial Sequence
  variant 13
MAAVSFTTLTTSGNWALPKVPSSQLQGRSMETRWHLLIMSAGATQYNFNGMMKFQPRTHQKIRSLSVDDSTNVVVMAERDNRENKSFGSMCIEDQITRINKLYLDIRMKFTSLSAYDTVHGGFPVNLIIGLSRVNHLESLGIGRHFKIEIKELLEDVFMFFLENEMVRAKPTASDIKTSSISFKILRYNGYDVSPDVFFSYVKDTDFVPWLENTGQATKAILQLYKASQMKWPGERILEEAKTFSQNYLENLKIGDQSGALMEIEADVMHALDVPWMAGVERVEHLRFMMAFAFDEVYVGETSSMVPFTGRDIFLSLAKQDFNICQEAQQEDLKGLEKWSVDSMVGDLHFGRQRLVACYFSAASTVWSPERSTTRIVWTKNAVITTVMDDFYDVEGSIEDINSFIDAVKRWDPLVLCNCSANLKKLFSAIYNSVQDIGQEIFVWQGFDISTNLREIYWELGITMKKEADWTMSGYLPTIEEYMENGRTSIALEPIIFSSLFWVGPKLTENMIHEYMKIRQIIQTCGRLINDSQSYKKEVKQGKLQSLSLFVREYPGSSLEDATEFIMVSLSETRQNVLRNLLQPSEVPRECKQIYFNKVKLLNVYYLHSDEYSSPTARIEHMNAVIWNPVV

SEQ ID NO: 54 - protein, 631 aa, Artificial Sequence
  variant 14
MGGISYSTVTTTAQYGIPMLPSTQVNAMSKDTKYHVLIKTAGASNWNFNGMRKYNPRTHQKVKTVSVEDTSQVLIKGDKENREKKSFGSMCIDDNITKINMLFVDIKKRWTSVSAYDTVHGAWPINLIIGLSKVNHLESLGIAMHFRVEIMDVLDDVFKYYIDNEMVRAMPSATDIMSSSISFKILRWQGYDVSPDIFFSYLKESEFLPWIDQSGQATRALLNLYKASNMMFPGERILEEAKTFSNNWLENIKVGDQSAALKDVDAEVMHGLDLPYMAGVDRIEHLRFMRAFGFDDIWVGETTSRIPWTGKDIFLSLAKQDFNICQEGQQEDLMGIDKWSVDSKLGELHFARQRLVACYWSAASSLWSPDKTTTRIVYTKQAILTSVKDDWYDIDGSIEDIQSFIEAVKRWDPILLCNCSANIKMLFSAIWQSINDIGQELWLYNGYDISTQVRDIFYELGISKMKEAEWTKSGFIPSLEEYMENGRTSIALEPIIWTSLFFLGPKLSEQMLHDYRKLMQVLNSCARLLNDSQSWMREVKQGMINSLSLFVKDWPGTSLEDATDFIKLTLSESRQNILKNLVNPSEIPRDCKQVWFNRIKVVQVFYIHTDEFTSPSAKLDHMQGLVWNPVI

SEQ ID NO: 55 - protein, 10 aa, Artificial Sequence
  Cup motif
ENNSFGSMCI

SEQ ID NO: 56 - protein, 10 aa, Artificial Sequence
  Cup motif
EKKSFGSMCI

SEQ ID NO: 57 - protein, 10 aa, Artificial Sequence
  Cup motif
EKNSFGSMCI

SEQ ID NO: 58 - protein, 10 aa, Artificial Sequence
  Cup motif
ENKSFGSMCI

(19) United States
(12) Patent Application Publication
(10) Pub. No.: US 2025/0002947 A1
(43) Pub. Date: Jan. 2, 2025

(54) RECOMBINANT MANUFACTURE OF C-20 TERPENOID ALCOHOLS

(71) Applicant: ISOBIONICS B.V., Geleen (NL)

(72) Inventors: Martinus Julius BEEKWILDER, Geleen (NL); Matthew Quinn STYLES, Geleen (NL); Hendrik Jan BOSCH, Wageningen (NL); Aurin Minnert VOS, Wageningen (NL); Adele Margaretha Maria Liduina VAN HOUWELINGEN, Wekerom (NL)

(21) Appl. No.: 18/576,769

(22) PCT Filed: Jun. 30, 2022
(86) PCT No.: PCT/EP2022/068104

§ 371 Date: Jan. 5, 2024

(30) Foreign Application Priority Data
Jul. 6, 2021 (EP) 21184067.3

(51) Int. Cl. C12P 5/00; C12N 9/88

(52) U.S. Cl. CPC C12P 5/007; C12N 9/88; C12Y 402/0314

(57) ABSTRACT

Disclosed is a method for the manufacture of at least one C-20 terpenoid alcohol comprising the steps of converting geranylgeranyl pyrophosphate into copalyl diphosphate (CPP) or labda-13-en-8-ol diphosphate (LPP) and converting CPP or LPP into at least one C-20 terpenoid alcohol, wherein said conversion is carried out by a polypeptide exhibiting diterpene alcohol synthase activity capable of converting CPP into manool, LPP into sclareol and/or LPP into abienol, and wherein said polypeptide comprises an amino acid sequence as specified in the claims. The invention further relates to the aforementioned polypeptide exhibiting diterpene alcohol synthase activity as well as a fusion protein comprising said polypeptide, a polynucleotide encoding it, a vector or gene construct comprising said polynucleotide, a host cell comprising said vector or gene construct. a non-human transgenic organism comprising the polynucleotide, vector, gene construct or host cell, as well as uses thereof for the manufacture of at least one C-20 terpenoid alcohol.

The present invention concerns the field of recombinant manufacture of C-20 terpenoid alcohols. In particular, it relates to a method for the manufacture of at least one C-20 terpenoid alcohol comprising the steps of converting geranylgeranyl pyrophosphate into copalyl diphosphate (CPP) or labda-13-en-8-ol diphosphate (LPP) and converting CPP or LPP into at least one C-20 terpenoid alcohol, wherein said conversion is carried out by a polypeptide exhibiting diterpene alcohol synthase activity wherein said diterpene alcohol synthase activity is capable of converting copalyl diphosphate (CPP) into manool, labda-13-en-8-ol diphosphate (LPP) into sclareol and/or LPP into abienol, wherein said polypeptide comprises and amino acid sequence selected from the group consisting of: a) an amino acid sequence as shown in any one of SEQ ID NOs: 3 to 7 or 34; b) an amino acid sequence which is at least 60%, at least 70%, at least 80%, at least 85%, at least 90%, at least 95%, at least 98% or at least 99% identical to the amino acid sequences as shown in any one of SEQ ID NOs: 3 to 7 or 34; c) an amino acid sequence encoded by a nucleic acid sequence as shown in SEQ ID NO: 1 or 2 or 35; d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1 or 2 or 35; and e) an amino acid sequence of a fragment of any one of (a) to (d), said fragment encoding a polypeptide exhibiting a diterpene alcohol synthase activity capable of converting copalyl diphosphate (CPP) into manool, labda-13-en-8-ol diphosphate (LPP) into sclareol and/or LPP into abienol. The invention further relates to the aforementioned polypeptide exhibiting diterpene alcohol synthase activity as well as a fusion protein comprising said polypeptide, a polynucleotide encoding it, a vector or gene construct comprising said polynucleotide, a host cell comprising said vector or gene construct, a non-human transgenic organism comprising the polynucleotide, vector, gene construct or host cell. Yet, the invention contemplates the use of said polypeptide, the fusion polypeptide, the polynucleotide, the vector or gene construct, the host cell or the non-human transgenic organism for the manufacture of at least one C-20 terpenoid alcohol, preferably, abienol, manool, and/or sclareol. Further, the invention encompasses a kit for the manufacture of at least one C-20 terpenoid alcohol, preferably, abienol, manool, and/or sclareol.

Sclareol ((+)-Sclareol), abienol (Z-abienol) and manool ((+)-manool) are members of the labdane diterpenes. Diterpenes are C-20 isoprenoids, and occur naturally in plants and microbes. These labdane diterpene molecules have commercial value since they can be converted into amber notes, which are applied in the fragrance industries. Examples of amber notes include amberketal, manool ketone, ambroxide and sclareolide. To convert the diterpene molecules to amber notes, several chemical or biocatalytic routes have been disclosed. Sclareol can be converted to ambroxide, e.g. Barrero et al. 1993, tetrahedron 49, 10405-10412; Farbood EP 0 204 009 B1), or to sclareolid (Farbood EP 0 419 026 A1). Manool can be converted to amberketal, e.g. U.S. Pat. No. 7,294,492 (Cryptococcus), or to manool ketone (EP 1 688 501 B1); abienol can be converted to ambroxide, (e.g. Barrero et al. 1993, tetrahedron 49, 10405-10412) or to sclareolide (U.S. Pat. No. 5,525,728).

Plant sources of these compounds include Salvia sclarea and Nicotiana glutinosa for sclareol; Halocarpus biformis (pink pine or yellow pine) for manool and Balsam fir (Abies balsamea) for abienol.

Genes encoding terpene cyclases for producing diterpenes have been extensively described (Zerbe, Trends Biotechnol 2015 July; 33(7):419-28.), and microbial production of these compounds has been demonstrated (e.g. Schalk J. Am. Chem. Soc. 2012, 134, 18900-18903). Diterpene biosynthesis starts from geranylgeranyl pyrophosphate (GGPP). GGPP is widely present in nature, as it is the precursor for carotenoids, plant hormones etc. GGPP synthases are widely known, and include e.g. the crtE from Synechococcus sp. PCC 7002, Saccharomyces cerevisiae, Mentha piperita, Arabidopsis thaliana (Feng Front. Plant Sci., 25 May 2020 and references therein), but also the idsA gene from Corynebacterium glutamicum (Heider FEBS Journal 281 (2014) 4906-4920).

Starting from GGPP, diterpene biosynthesis is usually mediated by two steps: Step 1 towards a cyclized diphosphate (e.g. labda-13-en-8-ol diphosphate or LPP, copalyl-PP or CPP), and step 2 for converting this substrate to the final product. Step 1 is usually carried out by a type II diterpene synthase, while step 2 is carried out by a type I diterpene synthase. There are type II synthases known which carry out both steps, such as the abienol synthase from Abies balsamea (Zerbe JOURNAL OF BIOLOGICAL CHEMISTRY VOL. 287, NO. 15, pp. 12121-12131, Apr. 6, 2012). Step I enzymes are usually alpha beta gamma domain proteins, characterized by the presence of a DXDD motif in the gamma domain. Step 2 enzymes can be alpha beta gamma domain proteins or alpha beta domain proteins, and are characterized by the presence of a DDXXD motive in the beta domain. Review on diterpene synthases is in Zerbe et al., Trends in Biotechnology, 2015, 33 (7), 419-428.

For biosynthesis of relevant diterpenes, the following genes have been described: For sclareol, LPP synthase (LPPS) and sclareol synthase (SS) from Salvia sclarea (Caniard et al. BMC Plant Biology 2012, 12:119; Schalk WO 2009/101126), LPPS is an alpha beta gamma protein (Type II), SS is an alpha beta protein (Type I), Ignea et al (Metabolic Engineering 27 (2015), 65-75) has demonstrated sclareol synthesis in yeast with only an LPPS, and similar enzymes from Nicotiana glutinosa (Julien, WO 2014/022434A1).

For abienol, LPPS and ABS from nicotiana tabacum (Salaud, The Plant Journal (2012) 72, 1-17; WO 2008/07031A1), Abies balsamea ABS, which can do both steps (Zerbe JOURNAL OF BIOLOGICAL CHEMISTRY VOL. 287, NO. 15, pp. 12121-12131, Apr. 6, 2012), and Abies ABS and Nicotiana ABS or salvia SS (WO2016/94178A1).

For manool, step 1 CPPS from Triticum aestivum, or Salvia Miltiorrhiza, or Talaromyces verruculosus or Coleus Forskohlii, Marrubium vulgare, Rosmarinus officinale; with step 2 salvia SS (US2019/0352673), Step 1 CPPS from Coleus forskohlii, with step 2 OmTPS4 from Origanum majorana (Johnson J. Biol. Chem. (2019) 294 (4) 1349-1362; WO 2020/028795).

Engineering microbes for producing sclareol, manool or abienol have been described. This includes the introduction of the following genetic elements:

A GGPP synthase was selected for example from the group of GGPP synthase described in Feng Front. Plant Sci., 25 May 2020. Also, CrtE type microbial enzymes have been employed for the purpose of generating GGPP, e.g. crtE from Pantoea agglomerans (AAA24819) (Schalk J. Am. Chem. Soc. 2012, 134, 18900-18903). Corynebacterium IdsA was shown to have a very high catalytic efficiency (Heider FEBS Journal 281 (2014) 4906-4920).

A step 1 gene, leading to LPP or (+)-CPP, was selected in the prior art from different sources. LPPS from Salvia sclarea (Caniard et al. BMC Plant Biology 2012, 12:119; Schalk WO 2009/101126), Nicotiana glutinosa (WO 2014/022434 Allylix), CfLPPS from Coleus forskohlii (Pateraki Plant Physiol., 164, 1222-1236; WO 2015/091943), NtLPPS from Nicotiana tabacum (Salaud, The Plant Journal (2012) 72, 1-17; WO 2008/07031A1), an GhLPPS from Grindelia hirsutula, an TwLPPS from Tripterygium wilfordii, a CcLPPS from Cistus creticus (Falara, Plant Physiology, 2010, Vol. 154, pp. 301-310). CPPS from Triticum aestivum, or Salvia Miltiorrhiza, or Talaromyces verruculosus or Coleus Forskohlii, Marrubium vulgare, Rosmarinus officinale (US2019/0352673) has been used as well.

Ma and co-workers describe the biochemical characterization of diterpene synthases of Taiwania cryptomerioides (Ma Li-Ting et al., The Plant Journal, vol. 100, no. 6, 1254-1272). Specifically, five monofunctional diTPS functions not previously observed in gymnosperms were characterized, including monofunctional class-II enzymes forming labda-13-en-8-ol diphosphate (LPP, TcCPS2) and (+)-copalyl diphosphate (CPP, TcCPS4), and three class-I diTPSs producing biformene (TcKSL1), levopimaradiene (TcKSL3) and phyllocladanol (TcKSL5), respectively. Yet, none of these diterpene synthases showed diterpene alcohol synthase activity, let alone the production of sclareol, manool or abienol.

Indeed, step 2 genes which lead to sclareol, manool or abienol are rare. Salvia sclarea sclareol synthase is known to produce manool when combined with CPPS (US2019/0352673). OmTPS4 from Origanum majorana is a manool synthase with CPPS, but with LPPS does not make sclareol, but makes manoyloxide (Johnson 2019). Jia (ACS Catal. 2018, 8, 3133-3137) discloses that salvia sclareol synthase can be converted to an isoabienol synthase by mutation of residue N431 to I, D or E: it can be changed from an 13R-sclareol synthase to a 13S-sclareol synthase by mutation N431Q. They claim sclareol synthase is exceptional in having an asparagine (N431), in a product-outcome-determining region around that residue, which is key for adding water to labdanoyl-PP to form sclareol. N. tabacum abienol synthase produces Z-biformene with CPPS from Salvia fruticosa. Jia et al have performed an alignment of sclareol synthase from Salvia sclarea with a number of step 2 diterpene synthase synthases from different species, including manoyl oxide synthase from Coleus forskohlii (GenBank accession: KF444508); 1 IrMS, miltiradiene synthase from Isodon rubescens (KX831652); CfMS, miltiradiene synthase from C. forskohlii (KF444509); RoMS1, miltiradiene synthase 1 from Rosemarius officinalis (KF805858); SmMS, miltiradiene synthase from Salvia miltiorrhiza (ABV08817); RoMS1, miltiradiene synthase from Rosemarius officinalis (KF805859); SfMS, miltiradiene synthase from Salvia fruticosa (KP091841); MvELS, 9,13-epoxy-labd-14-ene synthase from Marrubium vulgare (KJ584454). It was reported that the residue N438 of SsSS determines the ability to produce a 13-hydroxylated labdane diterpene, such as sclareol or manool.

Although various step 2 enzyme encoding genes have been reported in the prior art, there is nevertheless a need for highly efficient enzymes that can be applied for catalysing a step 2 reaction in the manufacture of C-20 terpenoid alcohols and, in particular, for abienol, sclareol and/or manool. Moreover, it would be desirable to have enzymes that are not limited to the production of only one C-20 terpenoid alcohol.

The technical problem underlying the present invention shall be seen as the provision of means and methods complying with the aforementioned needs. The technical problem is solved by the embodiments characterized in the claims and herein below.

Thus, the present invention relates to a method for the manufacture of at least one C-20 terpenoid alcohol comprising the steps of:
a) converting geranylgeranyl pyrophosphate into copalyl diphosphate (CPP) or labda-13-en-8-ol diphosphate (LPP); and
b) converting CPP or LPP into at least one C-20 terpenoid alcohol, wherein said conversion is carried out by a polypeptide exhibiting diterpene alcohol synthase activity wherein said diterpene alcohol synthase activity is capable of converting copalyl diphosphate (CPP) into manool, labda-13-en-8-ol diphosphate (LPP) into sclareol and/or LPP into abienol,
wherein said polypeptide comprises and amino acid sequence selected from the group consisting of:
a) an amino acid sequence as shown in any one of SEQ ID NOs: 3 to 10 or SEQ ID NO: 34;
b) an amino acid sequence which is at least 60%, at least 70%, at least 80%, at least 85%, at least 90%, at least 95%, at least 98% or at least 99% identical to the amino acid sequences as shown in any one of SEQ ID NOs: 3 to 10 or SEQ ID NO: 34;
c) an amino acid sequence encoded by a nucleic acid sequence as shown in SEQ ID NO: 1, 2, 16, 17, 18 or SEQ ID NO: 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1, 2, 16, 17, 18 or SEQ ID NO: 35; and
e) an amino acid sequence of a fragment of any one of (a) to (d), said fragment encoding a polypeptide exhibiting a diterpene alcohol synthase activity capable of converting copalyl diphosphate (CPP) into manool, labda-13-en-8-ol diphosphate (LPP) into sclareol and/or LPP into abienol.

It is to be understood that in the specification and in the claims, “a” or “an” can mean one or more of the items referred to in the following depending upon the context in which it is used. Thus, for example, reference to “an” item can mean that at least one item can be utilized.

As used in the following, the terms “have”, “comprise” or “include” are meant to have a non-limiting meaning or a limiting meaning. Thus, having a limiting meaning these terms may refer to a situation in which, besides the feature introduced by these terms, no other features are present in an embodiment described, i.e. the terms have a limiting meaning in the sense of “consisting of” or “essentially consisting of”. Having a non-limiting meaning, the terms refer to a situation where besides the feature introduced by these terms, one or more other features are present in an embodiment described.

Further, as used in the following, the terms “preferably”, “more preferably”, “most preferably”, “particularly”, “more particularly”, “typically”, and “more typically” are used in conjunction with features in order to indicate that these features are preferred features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five or any other number. Depending on the item the term refers to the skilled person understands as to what upper limit the term may refer, if any.

The method according to the present invention may either consist of steps (a) and (b) referred to above or may comprise additional steps. Such additional steps may be steps of pre-treatments or steps required for the manufacture of C-20 terpenoid alcohols such as purification steps.

The term “manufacture” as used herein refers to the generation of at least one C-20 terpenoid alcohol, in particular, a cyclic C-20 terpenoid alcohol more preferably, manool, sclareol and/or abienol, from CPP or LPP (CAS number 1000876-36-7). The manufacture may yield any degree of purity of the said at least one C-20 terpenoid alcohol. The higher the degree of envisaged purity, the more additional purification will be required. The method may be carried out ex-vivo, e.g., in one or more reaction vials. Alternatively, the method may be carried out entirely or in part in an organism such as a microorganism including the host cells referred to herein elsewhere or a non-human transgenic organism including plants.

The term “C-20 terpenoid alcohol” as used in accordance with the present invention relates to a C-20 terpenoid comprising an alcohol moiety. Terpenes are polymeric isoprenes. Terpenoids may have further functional chemical moieties. The C-20 terpenoids are also referred to as diterpenoids or diterpenes. Preferably, said at least one C-20 terpenoid alcohol referred to in accordance with the present invention is a cyclic C-20 terpenoid alcohol. More preferably, it is manool (CAS number 596-85-0, molecular formula C₂₀H₃₄O), sclareol (CAS number 515-03-7, molecular formula C₂₀H₃₆O₂) or abienol (CAS number 17990-16-8, molecular formula C₂₀H₃₄O).

The term “polypeptide” as used in accordance with the present invention refers to contiguous sequence of amino acid linked to each other by peptide bounds. A polypeptide according to the invention, typically, comprises at least 50, at least 100 or at least 200 amino acids in length such that the amino acid chain may form a three-dimensional structure required to exert the enzymatic activity or enzymatic activities referred to elsewhere herein. The term “protein” may be used interchangeably herein.

The term “diterpene alcohol synthase activity” as used to herein refers to an activity of the enzyme that allows for converting a starting material such as LPP or CPP into a C-20 terpenoid alcohol. Diterpene synthases undergo complex electrophilic cycle formations and/or rearrangements leading to diverse backbone structures. The diterpene synthases can be classified into class I enzymes which use terpene diphosphates as substrates that are generated from geranylgeranyl phosphate from the class II enzymes. The polypeptide having diterpene alcohol synthase activity referred to above is, typically, a type I enzyme. Preferably, said diterpene alcohol synthase activity is capable of converting copalyl diphosphate (CPP) into manool, labda-13-en-8-ol diphosphate (LPP) (CAS number 1000876-36-7) into sclareol and/or LPP (CAS number 1000876-36-7) into abienol. Preferably, the polypeptide having diterpene alcohol synthase activity in accordance with the present invention comprises a conserved region as shown in SEQ ID NO: 24 or a sequence with one or several amino acid changes to SEQ ID NO: 24, wherein the Serine at position 4 of SEQ IDNO: 24 is conserved or replaced by a Threonine; preferably the Serine at this position is conserved.

In addition, the polypeptide having diterpene alcohol synthase activity in accordance with the present invention comprises the Pfam domains PF01397.23 (Terpene synthase, N-terminal domain), PF03936.18 (Terpene synthase family, metal binding domain) and PF19086.2 (Terpene synthase family 2, C-terminal metal binding) (PFAM version 35.0); see Pfam: The protein families database in 2021: J. Mistry, S. Chuguransky, L. Williams, M. Qureshi, G.A. Salazar, E.L.L. Sonnhammer, S.C.E. Tosatto, L. Paladin, S. Raj, L.J. Richardson, R.D. Finn, A. Bateman Nucleic Acids Research (2020) doi: 10.1093/nar/gkaa913.

The polypeptide exhibiting according to the present invention diterpene alcohol synthase activity, wherein said diterpene alcohol synthase activity is capable of converting copalyl diphosphate (CPP) into manool, labda-13-en-8-ol diphosphate (LPP) into sclareol and/or LPP into abienol, comprises an amino acid sequence selected from the group consisting of:
a) an amino acid sequence as shown in any one of SEQ ID NOs: 3 to 10 or SEQ ID NO: 34;
b) an amino acid sequence which is at least 60%, at least 70%, at least 80%, at least 85%, at least 90%, at least 95%, at least 98% or at least 99% identical to the amino acid sequences as shown in any one of SEQ ID NOs: 3 to 10 or SEQ ID NO: 34;
c) an amino acid sequence encoded by a nucleic acid sequence as shown in SEQ ID NO: 1, 2, 16, 17, 18 or 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1, 2, 16, 17, 18 or 35; and
e) an amino acid sequence of a fragment of any one of (a) to (d), said fragment encoding a polypeptide exhibiting a diterpene alcohol synthase activity capable of converting copalyl diphosphate (CPP) into manool, labda-13-en-8-ol diphosphate (LPP) into sclareol and/or LPP into abienol.

Preferably, said polypeptide exhibiting diterpene alcohol synthase activity is capable of converting CPP into manool and LPP into sclareol. More preferably, said polypeptide comprises an amino acid sequence selected from the group consisting of:
a) an amino acid sequence as shown in SEQ ID NOs: 4, 6, 7, 9, 10 or SEQ ID NO: 34;
b) an amino acid sequence which is at least 60%, at least 70%, at least 80%, at least 85%, at least 90%, at least 95%, at least 98% or at least 99% identical to the amino acid sequences as shown in SEQ ID NOs: 4, 6, 7, 9, 10 or SEQ ID NO: 34;
c) an amino acid sequence encoded by a nucleic acid sequence as shown in SEQ ID NO: 2, 16, 17 or 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 2, 16, 17 or 35; and
e) an amino acid sequence of a fragment of any one of (a) to (d), said fragment encoding a polypeptide exhibiting a diterpene alcohol synthase activity capable of converting CPP into manool and LPP into sclareol.

Also preferably, said polypeptide exhibiting diterpene alcohol synthase activity is capable of converting LPP into abienol. More preferably, said polypeptide comprises an amino acid sequence selected from the group consisting of:
a) an amino acid sequence as shown in SEQ ID NO: 3, 5 or 8;
b) an amino acid sequence which is at least 60%, at least 70%, at least 80%, at least 85%, at least 90%, at least 95%, at least 98% or at least 99% identical to the amino acid sequences as shown in SEQ ID NO: 3, 5 or 8;
c) an amino acid sequence encoded by a nucleic acid sequence as shown in SEQ ID NO: 1 or 18;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1 or 18; and
e) an amino acid sequence of a fragment of any one of (a) to (d), said fragment encoding a polypeptide exhibiting a diterpene alcohol synthase activity capable of converting LPP into abienol.

The sequence identity referred to herein above defines a relationship between amino acid sequences or nucleic acid sequences and can be determined by comparing those sequences. Usually, sequence identities are determined by comparing two sequences over the whole length of the sequences but may also be compared only for a part of the sequences aligning with each other. Preferably, the sequence identities are compared over the whole length of the sequences, herein. Sequence identity refers to the degree of relatedness between polypeptide sequences or nucleic acid sequences. It will be expressed in the percentage of identical amino acids or nucleotides in two sequences compared to each other. Accordingly, upon aligning two sequences, the number of matching amino acids or nucleotides between those sequences is, in general, determined and put into relation to the total number of amino acids or nucleotides in the aligned sequence or sequence part. For instance, variant sequences may be defined by their sequence identity when compared to a parent sequence, i.e. an amino acid sequence as shown in any one of SEQ ID Nos: 3 to 7 or SEQ ID NO: 34, or a nucleic acid sequence as shown in SEQ ID NO: 1 or 2 or 35. To determine the percent-identity between two sequences in a first step a pairwise sequence alignment is generated between those two sequences, wherein the two sequences are aligned over their complete, entire or full length (i.e., a pairwise global alignment). The alignment is generated with a program or software described herein. The preferred alignment for the purpose of this invention is that alignment, from which the highest sequence identity can be determined.

Sequence alignments can be generated with a number of software tools, such as Needleman and Wunsch algorithm—Needleman, Saul B. & Wunsch, Christian D. (1970). “A general method applicable to the search for similarities in the amino acid sequence of two proteins”. Journal of Molecular Biology 48 (3): 443-453. This algorithm is, for example, implemented into the “NEEDLE” program, which performs a global alignment of two sequences. The NEEDLE program, is contained within, for example, the European Molecular Biology Open Software Suite (EMBOSS). EMBOSS—a collection of various programs: The European Molecular Biology Open Software Suite (EMBOSS), Trends in Genetics 16 (6), 276 (2000). BLOSUM (BLOcks Substitution Matrix)—typically generated on the basis of alignments of conserved regions, e.g., of protein domains (Henikoff S, Henikoff JG: Amino acid substitution matrices from protein blocks. Proceedings of the National Academy of Sciences of the USA. 1992 Nov. 15; 89(22): 10915-9). One out of the many BLOSUMs is “BLOSUM62”, which is often the “default” setting for many programs, when aligning protein sequences. BLAST (Basic Local Alignment Search Tool)—consists of several individual programs (BlastP, BlastN) which are mainly used to search for similar sequence in large sequence databases. BLAST programs also create local alignments. Typically used is the “BLAST” interface provided by NCBI (National Centre for Biotechnology Information), which is the improved version (“BLAST2”). The “original” BLAST: Altschul, S.F., Gish, W., Miller, W., Myers, E.W. & Lipman, D.J. (1990) “Basic local alignment search tool.” J. Mol. Biol. 215:403-410; BLAST2: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.

Sequence identity as used herein is, preferably, the value as determined by the EMBOSS Pairwise Alignment Algorithm “Needle”. In particular, the NEEDLE program from the EMBOSS package can be used (version 2.8.0 or higher, EMBOSS: The European Molecular Biology Open Software Suite-Rice, P., et al. Trends in Genetics (2000) 16:276-277; http://emboss.bioinformatics.nl) using the NOBRIEF option (‘Brief identity and similarity’ to NO) which calculates the “longest-identity”. The identity between the two aligned sequences is calculated in such a case as follows: Number of corresponding positions in the alignment showing an identical amino acid in both sequences divided by the total length of the alignment after subtraction of the total number of gaps in the alignment. For alignment of amino acid sequences the default parameters are: Matrix=Blosum62; Open Gap Penalty=10.0; Gap Extension Penalty=0.5. For alignment of nucleic acid sequences the default parameters are: Matrix=DNAfull; Open Gap Penalty=10.0; Gap Extension Penalty=0.5.

Variant amino acid or nucleic acid sequences as referred to herein may be naturally occurring variations such as allelic variants or othologous, paralogous or homologous variants. Alternatively, such sequences may be artificially generated, e.g., in an attempt to improve a property of the enzyme or nucleic acid (e.g., improved expression of the enzyme or increased enzymatic activity of the enzyme) by a biological technique known to the skilled person in the art, such as, e.g., molecular evolution or rational design, or by using a mutagenesis technique known in the art and described elsewhere herein (random mutagenesis, site-directed mutagenesis, directed evolution, gene recombination, etc.).

Variant nucleic acid sequences encoding an amino acid sequence encoded by a nucleic acid sequence as shown in SEQ ID NO: 1, 2, 16, 17, 18 or 35, or an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1, 2, 16, 17, 18 or 35 may differ from the nucleic acid sequences shown in SEQ ID NO: 1, 2, 16, 17, 18 or 35 for reasons set forth elsewhere herein due to at least one nucleotide substitution, addition and/or deletion. It will be understood that polynucleotides comprising such variant nucleic acid sequences as referred to herein, preferably, are capable of hybridizing to each other under stringent hybridization conditions. Stringent hybridization conditions as referred to herein are, preferably, 6× sodium chloride/sodium citrate (SSC) at approximately 45° C., followed by one or more wash steps in 0.2× SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5× SSC (pH 7.2). If organic solvent is present in the abovementioned buffer, for example 50% formamide, the temperature under standard conditions is approximately 42° C. The hybridization conditions for DNA: DNA hybrids are, preferably, 0.1× SSC and 20° C. to 45° C., preferably between 30° C. and 45° C. The hybridization conditions for DNA: RNA hybrids are, preferably, 0.1× SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Thus, variant nucleic acid sequences can be derived from polynucleotides which are capable of hybridizing under stringent hybridization conditions to nucleic acid sequences encoding an amino acid sequence encoded by a nucleic acid sequence as shown in SEQ ID NO: 1, 2, 16, 17, 18 or 35, or an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1, 2, 16, 17, 18 or 35.

In a further embodiment, the polypeptides of the invention comprise conserved amino acids at the positions indicated in FIG. 5 or 6, and preferably those given in FIG. 6. Conserved amino acid positions are indicated in FIGS. 5 and 6 by letters in white font on black background.

It was found that the polypeptides exhibiting diterpene alcohol synthase activity of the invention typically comprise a series of amino acids in the N-terminal area that in one letter code is EKKSFGSMCI (SEQ ID NO: 56) or ENKSFGSMCI (SEQ ID NO: 58) or ENNSFGSMCI (SEQ ID NO: 55) or EKNSFGSMCI (SEQ ID NO: 57). Preferably, the inventive polypeptides comprise the sequence as shown in SEQ ID NO: 56 or 58. The replacement of the first Lysine in this sequence stretch by an Asparagine, or replacing the Asparagine in this sequence stretch by a Lysine, respectively, did not have a significant impact on performance of the enzyme in the production of the at least one C-20 terpenoid alcohol, as referred to herein.

A fragment of the polypeptides exhibiting diterpene alcohol synthase activity of the invention may be a polypeptide consisting of any amino acid sequence of the above-mentioned sequences and sequence variants that is of sufficient length of exhibiting a diterpene alcohol synthase activity specified above. In this context, a conserved region has of the polypeptide referred to above has been identified in accordance with the present invention. This region (shown in SEQ ID NO: 24 or a sequence with one or several amino acid changes to SEQ ID NO: 24 wherein the Serine at position 4 of SEQ ID NO: 24 is conserved or replaced by a Threonine—preferably said Serine is conserved—is located from amino acid 486 to amino acid 497 in SEQ ID NO: 3 or from amino acid 486 to amino acid 497 in SEQ ID NO: 4. This region in the polypeptide according to the present invention exhibiting diterpene alcohol synthase activity is different from homologous, product determining regions in other synthases and, in particular, from the known Salvia sclareol synthase. It is, thus, preferably envisaged that a fragment having the aforementioned biological activity of the polypeptide comprises the amino acid sequence of a conserved product-outcome-determining region as specified above. Typically, a fragment comprises or consists of at least 20, at least 30, at least 40, at least 50, at least 100, at least 150, or at least 200 contiguous amino acids in length from the above-mentioned sequences or sequence variants of the invention and provides diterpene alcohol synthase activity.

The aforementioned polypeptide exhibiting diterpene alcohol synthase activity, wherein said diterpene alcohol synthase activity is capable of converting copalyl diphosphate (CPP) into manool, labda-13-en-8-ol diphosphate (LPP) into sclareol and/or LPP into abienol, may also be comprised in a fusion polypeptide. Such a fusion polypeptide comprises, in addition to the amino acid sequence of the polypeptide exhibiting diterpene alcohol synthase activity, one or more additional amino acid sequences. Said additional amino acid sequences may be, e.g., polypeptides having other enzymatic activities, such as type II diterpene synthase activity for catalysing step 1, polypeptides having support functions for the function of the polypeptide exhibiting diterpene synthase activity, or polypeptides or peptides having marker or label functions for, e.g., monitoring proper expression or for purification purposes, such as tags (e.g., MYC tag, FLAG tag, His tag, etc.) or fluorescent proteins (e.g., GFP, BFP, YFP or CFP).

Further, the present disclosure is directed to a method for preparing a C-20 terpenoid alcohol, preferably manool, sclareol and/or abienol, the method comprising converting copalyl diphosphate (CPP) and/or labda-13-en-8-ol diphosphate (LPP), respectively, into the C-20 terpenoid alcohol, preferably manool, sclareol and/or abienol, in the presence of an enzyme, the enzyme comprising a first segment comprising a tag peptide and a second segment comprising a diterpene alcohol synthase according to the invention. An enzyme comprising said first and said second segment may herein be referred to as a ‘tagged enzyme’.

The tag-peptide is preferably selected from the group of nitrogen utilization proteins (NusA), thioredoxins (Trx), maltose-binding proteins (MBP), Glutathione S-transferases (GST), Small Ubiquitin-like Modifier (SUMO) or Calcium-binding proteins (Fh8), and functional homologues thereof. As used herein, a functional homologue of a tag peptide is a tag peptide having at least about the same effect on the solubility of the tagged enzyme, compared to the non-tagged enzyme. Typically, the homologue differs in that one or more amino acids have been inserted, substituted, deleted from, or extended to the peptide of which it is a homologue. The homologue may in particular comprise one or more substitutions of a hydrophilic amino acid for another hydrophilic amino acid, or of a hydrophobic amino acid for another. The homologue may, in particular, have a sequence identity of at least 40%, more in particular of at least 50%, preferably of at least 55%, more preferably of at least 60%, at least 70%, at least 75%, at least 80%, at least 85%, at least 90%, at least 95%, at least 98% or at least 99% sequence identity with the sequence of a NusA, Trx, MBP, GST, SUMO or Fh8.

Particularly suitable is maltose-binding protein from Escherichia coli, or a functional homologue thereof.

The use of a tagged enzyme according to the invention is in particular advantageous in that it may contribute to an increased production, especially increased cellular production of a terpenoid or a terpene, such as C-20 terpenoid alcohol, preferably manool, sclareol and/or abienol.

For improved solubility of the tagged enzyme (compared to the enzyme without the tag), the first segment of the enzyme is preferably bound at its C-terminus to the N-terminus of the second segment. Alternatively, the first segment of the tagged enzyme is bound at its N-terminus to the C-terminus of the second segment.

Further, the present invention is directed to a nucleic acid comprising a nucleotide sequence encoding a polypeptide, the polypeptide comprising a first segment comprising a tag-peptide, preferably an MBP, a NusA, a Trx, a GST, a SUMO or anFh8-tag or a functional homologue of any of these, and a second segment comprising a diterpene alcohol synthase. The second segment may for instance comprise an amino acid sequence as shown in any one of SEQ ID NO: 3 to 7, 28 to 30, 34, or 40 to 54, or a functional analogue thereof.

Further, the present invention is directed to a host cell comprising said nucleic acid encoding said tagged diterpene alcohol synthase. Specific nucleic acids according to the invention encoding a tagged enzyme are shown in any one of SEQ ID NO: 8 to SEQ ID NO: 10 and SEQ ID NO: 28 to 30. The host cell may in particular comprise a gene comprising any of these sequences or a functional analogue thereof.

Further, the present invention is directed to an enzyme, comprising a first segment comprising a tag-peptide and a second segment comprising a polypeptide having enzymatic activity for converting a polyprenyl diphosphate into a terpene, in particular a diterpene alcohol synthase, the tag-peptide preferably being selected from the group of MBP, NusA, Trx or SET. Specific enzymes comprising a tagged enzyme according to the invention are shown in any one of SEQ ID NO: 8 to SEQ ID NO: 10, and SEQ ID NO: 28 to 30.

Preferably, a fusion protein shall further comprise a polypeptide which exhibits an enzymatic activity of a type II diterpene synthase. The conversion in step a) is carried out by a further polypeptide which exhibits an enzymatic activity of a type II diterpene synthase converting geranylgeranyl pyrophosphate (GGP) into LPP and/or CPP. Accordingly, the polypeptide exhibiting diterpene synthase activity is, preferably, comprised in a fusion polypeptide comprising at least one further polypeptide which exhibits an enzymatic activity of a type II diterpene synthase, preferably, converting geranylgeranyl pyrophosphate (GGP) into LPP and/or CPP, which has maltose binding properties or which is thioredoxin or a thioredoxin fusion protein. More preferably, said further polypeptide is selected from the group consisting of: an LPP synthase, preferably, from Coleus forskohlii (CfLPPS) (Pateraki, Plant Physiol., 164, 1222-1236 (2014); WO 2015/091943) or Nicotiana tabacum (NtLPPS) (Salaud, The Plant Journal (2012) 72, 1-17; WO200807031A1), a CPP synthase, preferably, from Coleus forskohlii (CfCPPS) (Johnson, J. Biol. Chem. (2019) 294 (4) 1349-1362; WO2020028795), thioredoxin, and maltose binding protein (MBP).

In step a) of the method of the present invention, geranylgeranyl pyrophosphate is converted into copalyl diphosphate (CPP) or labda-13-en-8-ol diphosphate (LPP). The said conversion is, typically, carried out enzymatically. Enzymes that are capable of converting geranylgeranyl phosphate into CPP or LPP are well known in the art. Preferably, the conversion is carried out by a polypeptide which exhibits an enzymatic activity of a type II diterpene synthase converting geranylgeranyl pyrophosphate (GGP) into LPP and/or CPP, more preferably, an LPP synthase, preferably, from Coleus forskohlii (CfLPPS) or Nicotiana tabacum (NtLPPS), a CPP synthase, preferably, from Coleus forskohlii (CfCPPS). It will be understood that the polypeptide exhibiting type II diterpene synthase activity is comprised in a fusion polypeptide together with the polypeptide exhibiting diterpene alcohol synthase activity of the invention, as described elsewhere herein in more detail.

The aforementioned step a) may be carried out in vitro, i.e. in a suitable reaction vial containing all components required for the conversion as described above. The skilled person is well aware of how to adjust the reaction conditions such that the reaction will be carried out efficiently. For example, suitable buffers may be used to provide the components in an environment having a suitable pH and suitable salt concentrations. A suitable temperature in such a setting can be applied as well without further ado.

Alternatively, step a) may be carried out in a host cell as described elsewhere herein. It is to be understood that the host cell shall be capable of producing GGP as well as a type II converting enzyme as specified above. If necessary, the host cell needs to be genetically modified in order to express such a type II enzyme or other enzymes or proteins required for the GGP synthesis. The host cell shall be cultivated under conditions and for a time sufficient to allow expression of the aforementioned enzymes and for conversion of GGP into CPP and/or LPP. Particular preferred conditions are also described in the accompanying Examples, below.

Yet, step a) of the method of the present invention may also be carried out in an organism, typically a multi-cellular organism such as the transgenic non-human organism referred to elsewhere herein. Typically, said organism is genetically modified such that the type II enzymes required for conversion of GGP into CPP and/or LPP are expressed.

In step b) of the method of the present invention, CPP or LPP is converted into at least one C-20 terpenoid alcohol, wherein said conversion is carried out by a polypeptide exhibiting diterpene alcohol synthase activity wherein said diterpene alcohol synthase activity is capable of converting copalyl diphosphate (CPP) into manool, labda-13-en-8-ol diphosphate (LPP) into sclareol and/or LPP into abienol, preferably by a diterpene alcohol synthase of the invention.

Step b) of the method of the present invention may also be carried out in vitro or in a host cell or an organism as specified for step a) above. Particular preferred conditions are described in the accompanying Examples, below.

Preferably, said step b) or said steps a) and b) are carried out in a host cell or in a non-human transgenic organism. More preferably, said host cell or non-human transgenic organism is a host cell or non-human transgenic organism of the invention as described elsewhere herein in more detail. It will be understood that the conditions which need to be applied for carrying out step b) or step a) and b) in a host cell or a non-human transgenic organism depend on the said host cell or non-human transgenic organism. The skilled person is, however, well aware of what conditions need to be applied depending on the choice of a given host cell or non-human transgenic organism.

Preferably, the method of the present invention comprises the step of obtaining said manufactured at least one C-20 terpenoid alcohol.

The term “obtaining” as used herein refers to providing the at least one C-20 terpenoid alcolhol at any degree of purity after step b). Accordingly, the at least one C-20 terpenoid alcohol may be provided in essentially pure form or as a composition comprising additional components. Thus, the method of the invention may encompass one or more purification steps, after step b) has been completed. The purification techniques which need to be applied depend on how the steps a) and/or b) of the method of the present invention have been carried out. For example, if these steps have been carried out in vitro, i.e. in reaction vials using isolated components such as isolated enzymes, adducts and auxiliary components such as reaction buffers, it will be understood that less purification is required in order to obtain an, e.g., essentially pure at least one C-20 terpenoid alcohol. However, if steps a) and b) are carried out in vivo, i.e. in a host cell as defined elsewhere herein, further purification and pre-treatment steps may be necessary. Typically, the host cells need to be harvested and the harvested cells may have to be lysed in order to release the C-20 terpenoid alcohols from said cells. Subsequent purification steps shall remove the cell debris as well as aiming at purifying the C-20 terpenoid alcohol from the remaining components. Moreover, if the steps are carried out in vivo in animals or plants, even further pre-treatment and/or purification steps may be required in order to obtain the at least one C-20 terpenoid alcohol. The skilled person is well aware of suitable pre-treatment and/or purification steps depending on the given circumstances under which steps a) and b) are carried out. Purification techniques to be envisaged may be extraction techniques, chromatography, such as LC, GC or HPLC, size-exclusion chromatography, affinity chromatography, distillation, centrifugation, filtration and the like. Pre-treatment steps to be envisaged may be harvesting, heat treatment, ultra-sonic treatment, treatment with chemicals and/or enzymes, and the like. Particular preferred measures are described in the accompanying Examples, below.

Advantageously, the studies underlying the present invention revealed that a family of step 2 enzymes from Cupressa gigantea, i.e. Cup2v1 and Cup2v2b, are capable of efficiently converting CPP and LPP into the C-20 terpenoid alcohols manool, sclareol and/or abienol. In particular, it was found that the Cup2v1 and Cup2v2b enzymes when expressed in, e.g., Rhodobacter, are particularly efficient in the recombinant manufacture of the C-20 terpenoid alcohols, as described in the accompanying Examples below. Moreover, it was found that the Cup2v2a and Cup2v2b enzymes, i.e. a polypeptide having an amino acid sequence as shown in any one of SEQ ID NOs: 4, 6, 7, 9, 10, or 34, or variants thereof as specified elsewhere herein, are capable of producing two C-20 terpenoid alcohols, i.e. manool and sclareol. Cup2v1, i.e. a polypeptide having an amino acid sequence as shown in SEQ ID NOs: 3, 5 or 8 or variants thereof as specified elsewhere herein, was efficient in the production of abienol.

Thanks to the present invention, C-20 terpenoid alcohols can be manufactured more efficiently, in particular, in recombinant manufacturing approaches.

In one embodiment, an enzyme is considered useful in the methods of the invention if the enzyme preferentially produces C-20 terpenoid alcohol(s). In a further embodiment, preferentially producing C-20 terpenoid alcohol(s) is to be understood that when the enzyme is provided with a large variety of substrates under conditions suitable for the enzyme to be active amongst the products produced by the enzyme, the C-20 terpenoid alcohol(s) is (are) dominant. For example, from all molecules produced by the enzyme, more than 50% of the molecules are C-20 terpenoid alcohol(s).

In another embodiment, an inventive polypeptide exhibiting diterpene alcohol synthase activity is characterized by the fact that it preferentially produces manool from CPP, and/or sclareol from LPP and/or abienol from LPP.

In a further embodiment, preferentially producing manool, sclareol and/or abienol is to be understood that when the enzyme is provided with a suitable substrate, for example LPP or CPP, under conditions suitable for the enzyme to be active amongst the products produced by the enzyme, the manool, sclareol and/or abienol are dominant. For example, from all molecules produced by the enzyme, more than 50% of the molecules are any of these: manool, sclareol or abienol.

The present invention further relates to a method for the production of an aroma composition, comprising the steps of:
a) producing one or more C-20 terpenoid alcohol(s), preferably, abienol, manool, and/or sclareol, according to the method of the invention, preferably according to the method of any one of claims 1 to 5,
b) optionally purifying said one or more C-20 terpenoid alcohol(s), and
c) preparing or formulating an aroma composition with said one or more C-20 terpenoid alcohol(s).

An aroma composition as used herein can be, for instance, a flavour, a fragrance or a perfume; see, e.g., Chemistry and Technology of Flavors and Fragrances, Editor(s): David J. Rowe, First published: 26 Oct. 2004, Print ISBN: 9781405114509 |Online ISBN: 9781444305517 |DOI: 10.1002/9781444305517, Blackwell Publishing Ltd.

The definitions and explanations of the terms made herein before apply mutatis mutandis to the following embodiments of the present invention except if specified otherwise.

The present invention also provides a composition or an aroma composition comprising said at least one C-20 terpenoid alcohol, preferably, manool, sclareol and/or abienol, obtainable by the method of the present invention.

In addition, the invention pertains to a composition comprising a host cell or a non-human transgenic organism, and said at least one C-20 terpenoid alcohol, preferably, manool, sclareol and/or abienol, obtainable by the method of the invention, preferably by the method of any one of claims 1 to 5, wherein the host cell or a non-human transgenic organism comprises recombinantly at least one polypeptide exhibiting diterpene alcohol synthase activity with
a) an amino acid sequence as shown in any one of SEQ ID NOs: 3 to 10 or SEQ ID NO: 34;
b) an amino acid sequence which is at least 60%, at least 70%, at least 80%, at least 85%, at least 90%, at least 95%, at least 98% or at least 99% identical to the amino acid sequences as shown in any one of SEQ ID NOs: 3 to 10 or SEQ ID NO: 34;
c) an amino acid sequence encoded by a nucleic acid sequence as shown in SEQ ID NO: 1, 2, 16, 17, 18 or 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1, 2, 16, 17, 18 or 35; and
e) an amino acid sequence of a fragment of any one of (a) to (d), said fragment encoding a polypeptide exhibiting a diterpene alcohol synthase activity capable of converting copalyl diphosphate (CPP) into manool, labda-13-en-8-ol diphosphate (LPP) into sclareol and/or LPP into abienol.

Yet, the present invention also relates to a polypeptide exhibiting diterpene alcohol synthase activity, wherein said diterpene alcohol synthase activity is capable of converting copalyl diphosphate (CPP) into manool, labda-13-en-8-ol diphosphate (LPP) into sclareol and/or LPP into abienol, said polypeptide having an amino acid sequence selected from the group consisting of:
a) an amino acid sequence as shown in any one of SEQ ID NOs: 3 to 10 or SEQ ID NO: 34;
b) an amino acid sequence which is at least 60%, at least 70%, at least 80%, at least 85%, at least 90%, at least 95%, at least 98% or at least 99% identical to the amino acid sequences as shown in any one of SEQ ID NOs: 3 to 10 or SEQ ID NO: 34;
c) an amino acid sequence encoded by a nucleic acid sequence as shown in SEQ ID NO: 1, 2, 16, 17, 18 or 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1, 2, 16, 17, 18 or 35; and
e) an amino acid sequence of a fragment of any one of (a) to (d), said fragment encoding a polypeptide exhibiting a diterpene alcohol synthase activity capable of converting copalyl diphosphate (CPP) into manool, labda-13-en-8-ol diphosphate (LPP) into sclareol and/or LPP into abienol.

Preferably, said diterpene alcohol synthase activity is capable of converting CPP into manool and LPP into sclareol. More preferably, said polypeptide comprises an amino acid sequence selected from the group consisting of:
a) an amino acid sequence as shown in SEQ ID NOs: 4, 6, 7, 9, 10 or 34;
b) an amino acid sequence which is at least 60%, at least 70%, at least 80%, at least 85%, at least 90%, at least 95%, at least 98% or at least 99% identical to the amino acid sequences as shown in SEQ ID NOs: 4, 6, 7, 9, 10 or 34;
c) an amino acid sequence encoded by a nucleic acid sequence as shown in SEQ ID NO: 2, 16, 17 or 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 2, 16, 17 or 35; and
e) an amino acid sequence of a fragment of any one of (a) to (d), said fragment encoding a polypeptide exhibiting a diterpene alcohol synthase activity capable of converting CPP into manool and LPP into sclareol.

Also preferably, said diterpene alcohol synthase activity is capable of converting LPP into abienol. More preferably, said polypeptide comprises an amino acid sequence selected from the group consisting of:
a) an amino acid sequence as shown in SEQ ID NO: 3, 5 or 8;
b) an amino acid sequence which is at least 60%, at least 70%, at least 80%, at least 85%, at least 90%, at least 95%, at least 98% or at least 99% identical to the amino acid sequences as shown in SEQ ID NO: 3, 5 or 8;
c) an amino acid sequence encoded by a nucleic acid sequence as shown in SEQ ID NO: 1 or 18;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1 or 18; and
e) an amino acid sequence of a fragment of any one of (a) to (d), said fragment encoding a polypeptide exhibiting a diterpene alcohol synthase activity capable of converting LPP into abienol.

The present invention also contemplates a fusion polypeptide comprising the polypeptide of the present invention and at least one further polypeptide (i) which exhibits an enzymatic activity of a type II diterpene synthase, preferably, converting geranylgeranyl pyrophosphate (GGP) into LPP and/or CPP, (ii) which has maltose binding properties or (iii) which is thioredoxin or a thioredoxin fusion protein. More preferably, said further polypeptide is selected from the group consisting of: an LPP synthase, preferably, from Coleus forskohlii (CfLPPS) or Nicotiana tabacum (NtLPPS), a CPP synthase, preferably, from Coleus forskohlii (CfCPPS), thioredoxin, and maltose binding protein (MBP).

The invention also relates to a method for producing the polypeptide having diterpene alcohol synthase activity of the invention, comprising
(a) transforming host cells or unicellular organisms with the nucleic acid sequence of the invention to express a polypeptide having diterpene alcohol synthase activity;
(b) obtaining or isolating from the host cell of step (a) said polypeptide having diterpene alcohol synthase activity; and
(c) optionally, purifying said polypeptide having diterpene alcohol synthase activity.

The invention further relates to a method for preparing a variant polypeptide having a diterpene alcohol synthase activity comprising the steps of:
a) selecting a nucleic acid of the invention or a nucleic acid encoding a polypeptide of the invention;
b) modifying the selected nucleic acid to obtain at least one mutant nucleic acid;
c) transforming host cells or unicellular organisms with the mutant nucleic acid sequence to express a polypeptide encoded by the mutant nucleic acid sequence;
d) screening the polypeptide for at least one modified property as well as diterpene alcohol synthase activity; and,
e) optionally, if the polypeptide has no desired variant diterpene alcohol synthase activity, repeating the process steps (a) to (d) until a polypeptide with a desired variant diterpene alcohol synthase activity is obtained;
f) optionally, if a polypeptide having a desired variant diterpene alcohol synthase activity was identified in step (d), isolating the corresponding mutant nucleic acid obtained in step (c).

The present invention relates to a polynucleotide encoding the polypeptide of the invention or the fusion polypeptide of the invention or a reverse complementary or complementary sequence thereof.

The term “polynucleotide” as used in accordance with the present invention refers to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). The term as used herein encompasses the sequence specified herein as well as the complementary or reverse-complementary sequence thereof. Thus, the term encompasses DNAs or RNAs with backbones modified for stability or for other reasons. Moreover, DNAs or RNAs comprising unusual bases, such as inosine, or modified bases, such as tritylated bases, to name just two examples, are also encompassed as polynucleotides. It will be appreciated that a great variety of modifications have been made to DNA and RNA that serve many useful purposes known to those of skill in the art. Every nucleic acid sequence herein that encodes a certain polypeptide of the invention may due to the degeneracy of the genetic code have silent variations. The degeneracy of the genetic code yields a large number of functionally identical polynucleotides that encode the same polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are silent variations.

The polynucleotide of the invention shall encode the polypeptide of the invention, i.e. it shall comprise a nucleic acid sequences which encodes said polypeptide of the invention. In addition, the polynucleotide of the present invention may comprise additional nucleic acid sequences. Preferably, the polynucleotide of the present invention may comprise in addition to an open reading frame further untranslated sequence at the 3′ and at the 5′ terminus of the coding gene region: at least 500, preferably 200, more preferably 100 nucleotides of the sequence upstream of the 5′ terminus of the coding region and at least 100, preferably 50, more preferably 20 nucleotides of the sequence downstream of the 3′ terminus of the coding gene region.

The polynucleotide of the present invention shall be provided, preferably, either as an isolated polynucleotide (i.e. purified or at least isolated from its natural context such as its natural gene locus) or in genetically modified or exogenously (i.e. artificially) manipulated form. An isolated polynucleotide can, for example, comprise less than approximately 5 kb, 4 kb, 3 kb, 2 kb, 1 kb, 0.5 kb or 0.1 kb of nucleotide sequences which naturally flank the nucleic acid molecule in the genomic DNA of the cell from which the nucleic acid is derived. The polynucleotide, preferably, is provided in the form of double or single stranded molecule. It will be understood that the present invention by referring to any of the aforementioned polynucleotides of the invention also refers to complementary or reverse complementary strands of the specific sequences or variants there-of referred to before. The polynucleotide encompasses DNA, including cDNA and genomic DNA, or RNA polynucleotides.

However, the present invention also pertains to polynucleotide variants which are derived from the polynucleotides of the present invention and are capable of interfering with the transcription or translation of the polynucleotides of the present invention. Such variant polynucleotides include anti-sense nucleic acids, ribozymes, siRNA molecules, morpholino nucleic acids (phosphorodiamidate morpholino oligos), triple-helix forming oligonucleotides, inhibitory oligonucleotides, or micro RNA molecules all of which shall specifically recognize the polynucleotide of the invention due to the presence of complementary or substantially complementary sequences. These techniques are well known to the skilled artisan. Suitable variant polynucleotides of the aforementioned kind can be readily designed based on the structure of the polynucleotides of this invention.

Moreover, comprised are also chemically modified polynucleotides including naturally occurring modified polynucleotides such as glycosylated or methylated polynucleotides or artificial modified ones such as biotinylated polynucleotides.

The present invention also relates to a vector or gene construct comprising the polynucleotide of the invention.

The term “vector”, preferably, encompasses phage, plasmid, cosmids, viral vectors as well as artificial chromosomes, such as bacterial or yeast artificial chromosomes (YAC). The vector encompassing the polynucleotide of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, f-mating, natural competence, carbon-based clusters, chemically mediated transfer, electroporation or particle bombardment. Suitable methods for the transformation or transfection of host cells, including plant cells, can be found in Sambrook et al. (Molecular Cloning: A Laboratory Manual, 2nd ed., Cold Spring Harbor Laboratory, Cold Spring Harbor Laboratory Press, Cold Spring Harbor, NY, 1989) and other laboratory manuals, such as Methods in Molecular Biology, 1995, Vol. 44, Agrobacterium protocols, Ed.: Gartland and Davey, Humana Press, Totowa, New Jersey. Alternatively, a plasmid vector may be introduced by heat shock or electroporation techniques. Should the vector be a virus, it may be packaged in vitro using an appropriate packaging cell line prior to application to host cells.

Preferably, the vector referred to herein is suitable as a cloning vector, i.e. replicable in microbial systems. Such vectors ensure efficient cloning in bacteria and, preferably, yeasts or fungi and make possible the stable transformation of plants. Those which must be mentioned are, in particular, various binary and co-integrated vector systems which are suitable for the T DNA-mediated transformation. Such vector systems are, as a rule, characterized in that they contain at least the vir genes, which are required for the Agrobacterium-mediated transformation, and the sequences which delimit the T-DNA (T-DNA border). These vector systems, preferably, also comprise further cis-regulatory regions such as promoters and terminators and/or selection markers with which suitable transformed host cells or organisms can be identified. While co-integrated vector systems have vir genes and T DNA sequences arranged on the same vector, binary systems are based on at least two vectors, one of which bears vir genes, but no T-DNA, while a second one bears T DNA, but no vir gene. As a consequence, the last-mentioned vectors are relatively small, easy to manipulate and can be replicated both in E. coli and in Agrobacterium. These binary vectors include vectors from the pBIB-HYG, pPZP, pBecks, pGreen series. Preferably used in accordance with the invention are Bin19, pBI101, pBinAR, pGPTV and pCAMBIA. An overview of binary vectors and their use can be found in Hellens et al, Trends in Plant Science (2000) 5, 446-451. Furthermore, by using appropriate cloning vectors, the polynucleotides can be introduced into host cells or organisms such as plants or animals and, thus, be used in the transformation of plants, such as those which are published, and cited, in: Plant Molecular Biology and Biotechnology (CRC Press, Boca Raton, Florida), chapter 6/7, pp. 71-119 (1993); F.F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus 1991, Annu. Rev. Plant Physiol. Plant Molec. Biol. 42, 205 225.

More preferably, the vector of the present invention is an expression vector. In such an expression vector, i.e. a vector which comprises the polynucleotide of the invention having the nucleic acid sequence operatively linked to an expression control sequence (also called “expression cassette”) allowing expression in prokaryotic or eukaryotic cells or isolated fractions thereof. Suitable expression vectors are known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX (Pharmacia Biotech Inc; Smith 1988, Gene 67:31-40), pMAL (New England Biolabs, Beverly, MA) and pRIT5 (Pharmacia, Piscataway, NJ), where glutathione S transferase (GST), maltose E-binding protein and protein A, respectively, are fused with the recombinant target protein. Examples of suitable inducible nonfusion E. coli expression vectors are, inter alia, pTrc (Amann 1988, Gene 69:301-315) and pET 11d (Studier 1990, Methods in Enzymology 185, 60-89). The tar-get gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a co-expressed viral RNA polymerase (T7 gn1). This viral polymerase is provided by the host strains BL21 (DE3) or HMS174 (DE3) from a resident labda-prophage which harbours a T7 gn1 gene under the transcriptional control of the lacUV 5 promoter. The skilled worker is familiar with other vectors which are suitable in prokaryotic organisms; these vectors are, for example, in E. coli, pLG338, pACYC184, the pBR series such as pBR322, the pUC series such as pUC18 or pUC19, the M113mp series, pKC30, pRep4, pHS1, pHS2, pPLc236, pMBL24, pLG200, pUR290, pIN-III113-B1, lambdagt11 or pBdCI, in Streptomyces pIJ101, pIJ364, pIJ702 or pIJ361, in Bacillus pUB110, pC194 or pBD214, in Corynebacterium pSA77 or pAJ667. Examples of vectors for expression in the yeast S. cerevisiae comprise pYep Sec1 (Baldari 1987, Embo J. 6:229-234), pMFa (Kurjan 1982, Cell 30:933-943), pJRY88 (Schultz 1987, Gene 54:113-123) and pYES2 (Invitrogen Corporation, San Diego, CA). Vectors and pro-cesses for the construction of vectors which are suitable for use in other fungi, such as the filamentous fungi, comprise those which are described in detail in: van den Hondel, C.A.M.J.J., & Punt, P.J. (1991) “Gene transfer systems and vector development for filamentous fungi, in: Applied Molecular Genetics of fungi, J.F. Peberdy et al., Ed., pp. 1-28, Cambridge University Press: Cambridge, or in: More Gene Manipulations in Fungi (J.W. Bennett & L.L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells (for example Sf9 cells) comprise the pAc series (Smith 1983, Mol. Cell Biol. 3:2156-2165) and the pVL series (Lucklow 1989, Virology 170:31-39).

Yet the vector may be an integration vector. An integration vector refers to a DNA molecule, linear or circular, that can be incorporated, e.g., into a microorganism's genome, such as a bacteria's genome, and provides for stable inheritance of a gene encoding a polypeptide of interest, such as the alcohol acyl transferase of the invention. The integration vector generally comprises one or more segments comprising a gene sequence encoding a polypeptide of interest under the control of (i.e., operably linked to) additional nucleic acid segments that provide for its transcription.

Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is non-functional in that organism. Integration of the segment comprising the gene of interest may be selected if an appropriate marker is included within that segment. One or more nucleic acid sequences encoding appropriate signal peptides that are not naturally associated with a polypeptide to be expressed in a host cell of the invention can be incorporated into (expression) vectors. For example, a DNA sequence for a signal peptide leader can be fused in-frame to a nucleic acid of the invention so that the alcohol acyl transferase of the invention is initially translated as a fusion protein comprising the signal peptide. Depending on the nature of the signal peptide, the expressed polypeptide will be targeted differently. A secretory signal peptide that is functional in the intended host cells, for instance, enhances extracellular secretion of the expressed polypeptide. Other signal peptides direct the expressed polypeptide to certain organelles, like the chloroplasts, mitochondria and peroxisomes. The signal peptide can be cleaved from the polypeptide upon transportation to the intended organelle or from the cell. It is possible to provide a fusion of an additional peptide sequence at the amino or carboxyl terminal end of the polypeptide.

The term “gene construct” as used herein refers to polynucleotides comprising the polynucleotide of the invention and additional functional nucleic acid sequences. A gene construct according to the present invention is, preferably, a linear DNA molecule. Typically, a gene construct in accordance with the present invention may be a targeting construct which allows for random or site-directed integration of the targeting construct into genomic DNA. Such target constructs, preferably, comprise DNA of sufficient length for either homologous or heterologous recombination as described in detail below. In both cases, the construct must be, preferably, impeccable, with structures to control gene expression, such as a promoter, a site of transcription initiation, a site of polyadenylation, and a site of transcription termination. Yet, the present invention relates to a host cell comprising the vector or gene construct of the invention.

The host cell of the invention is capable of expressing the polypeptide of the invention comprised in the vector or gene construct of the invention. The host cell is, typically transformed with said vector or gene construct such that the polypeptide of the invention can be expressed from the vector or gene construct. The transformed vector or gene construct may be maintained as a non-integrated vector, for example, a plasmid, or alternatively, may be integrated into the host cell genome as specified elsewhere herein in more detail.

A host cell according to the invention may be produced based on standard genetic and molecular biology techniques that are generally known in the art, e.g., as described in Sambrook, J., and Russell, D.W. “Molecular Cloning: A Laboratory Manual” 3d ed., Cold Spring Harbor Laboratory Press, Cold Spring Harbor, NY, (2001); and F.M. Ausubel et al, eds., “Current protocols in molecular biology”, John Wiley and Sons, Inc., New York (1987), and later supplements thereto.

Preferably, said host cell is selected from the group consisting of: a bacterial cell, a yeast cell, a fungal cell, an algal cell or a cyanobacterial cell, a non-human animal cell or a non-human mammalian cell, and a plant cell. More preferably, the host cell can be selected from any one of the following organisms:

### Bacteria

The bacterial host cell can, for example, be selected from the group consisting of the genera Escherichia, Klebsiella, Helicobacter, Bacillus, Lactobacillus, Streptococcus, Amycolatopsis, Rhodobacter, Pseudomonas, Paracoccus, Lactococcus or Pantoea.

gram positive: Bacillus, Streptomyces. Useful gram positive bacterial host cells include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus Jautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus.

Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

gram negative: E. coli, Pseudomonas, Rhodobacter, Paracoccus. Preferred gram negative bacteria are Escherichia coli, Pseudomonas sp., preferably, Pseudomonas purrocinia (ATCC 15958) or Pseudomonas fluorescens (NRRL B-11), Rhodobacter capsulatus or Rhodobacter sphaeroides, Paracoccus carotinifaciens, Paracoccus zeaxanthinifaciens or Pantoea ananatis.

### Fungi

Aspergillus, Fusarium, Trichoderma. The host cell may be a fungal cell. “Fungi” as used herein includes the phyla Ascomycota, Basidiomycota, Chytridiomycota, and Zygomycota as well as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g. Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucana (IFO 6113), Verticillum alboatrum, Verticillum dahlie, Arthromyces ramosus (FERM P-7754), Caldariomyces fumago, Ulocladium chartarum, Embellisia alli or Dreschlera halodes. Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A). Further preferred fungi include strains belonging to the subdivision Zygomycotina, class Mycoraceae, e.g. Rhizopus or Mucor, in particular Mucor hiemalis.

Yeast, Pichia, Saccharomyces: The fungal host cell may be a yeast cell. Yeast as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycesceae. The latter is comprised of four subfamilies, Schizosaccharomycoideae (e.g., genus Schizosaccharomyces), Nadsonioideae, Lipomycoideae, and Saccharomycoideae (e.g. genera Kluyveromyces, Pichia, and Saccharomyces). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sporobolomyces and Bullera) and Cryptococcaceae (e.g. genus Candida).

### Eukaryotes

Eukaryotic host cells further include, without limitation, a non-human animal cell, a non-human mammal cell, an avian cell, reptilian cell, insect cell or a plant cell.

Most preferably, the host cell is a bacterial host cell, in particular, a Rhodobacter host cell.

The present invention relates to a transgenic non-human organism comprising the polynucleotide of the invention, the vector or gene construct of the invention, or the host cell of the invention.

The term “transgenic non-human organism” as used herein refers to an organism which has been genetically modified in order to comprise the polynucleotide, vector or gene construct of the present invention. Said genetic modification may be the result of any kind of homologous or heterologous recombination event, mutagenesis or gene editing process. Accordingly, the transgenic non-human organism shall differ from its non-transgenic counterpart in that it comprises the non-naturally occurring (i.e. heterologous) polynucleotide, vector or gene construct in its genome. Non-human organisms envisaged as transgenic non-human organisms in accordance with the present invention are, preferably, multi-cellular organisms. Moreover, the non-human organisms are, preferably, animals or plants. Preferred animals are mammals, in particular laboratory animals such as rodents, e.g., mice, rats, rabbits or the like, or farming animals such as sheep, goat, cows, horses or the like. Preferred plants are crop plants or vegetables, in particular, selected from the group consisting of Arabidopsis spp., Nicotiana spp, Cichorum intybus, Lactuca sativa, Mentha spp, Artemisia annua, tuber forming plants, oil crops, e.g. Brassica spp. or Brassica napus, flowering plants (angiosperms) which produce fruits, and trees.

A non-human transgenic organism in one embodiment is a non-human transgenic organism that is transgenic for the polypeptide of the invention, for a fusion protein comprising said polypeptide, a polynucleotide encoding it, a vector or gene construct comprising said polynucleotide.

The host cell in one embodiment is a non-human cell in vitro, for example, in cell cultures.

In another embodiment, the term “non-human” is to be understood to refer to organisms other than humans that are not animals (for example plants, fungus or microorganisms) or are animals other than mammals, preferably animals that are not vertebrates.

Methods for the production of transgenic non-human organisms are well known in the art; see, e.g. Lee-Yoon Low et al., Transgenic Plants: Gene constructs, vector and transformation method. 2018. DOI.10.5772/intechopen.79369; Pinkert, C. A. (ed.) 1994. Transgenic animal technology: A laboratory handbook. Academic Press, Inc., San Diedo, Calif.; Monastersky G. M. and Robl, J. M. (ed.) (1995) Strategies in Transgenic Animal Science. ASM Press. Washington D.C); Sambrook, loc.cit, Ausubel, loc.cit).

The present invention, in general, contemplates the use of the polypeptide of the invention or the fusion polypeptide of the invention, the polynucleotide of the invention, the vector or gene construct of the invention, the host cell of the invention or the non-human transgenic organism of the invention for the manufacture of at least one C-20 terpenoid alcohol, preferably, abienol, manool, and/or sclareol.

The C-20 terpenoid alcohol which is manufactured according to the present invention may have a variety of utilities in different industrial sectors. In particular, the said C-20 terpenoid alcolhol is used for producing flavours, agrochemicals, fragrances, pharmaceutical compositions, cosmetics or chemical building blocks.

Moreover, the present invention also relates to a kit for the manufacture of at least one C-20 terpenoid alcohol, preferably, abienol, manool, and/or sclareol, comprising the polypeptide of the invention or the fusion polypeptide of the invention, the polynucleotide of the invention, the vector or gene construct of the invention, the host cell of the invention, or the non-human transgenic organism of the invention.

The term “kit” as used herein refers to a collection of components required for carrying out the method of the present invention for the manufacture of at least one C-20 terpenoid alcohol. The kit shall include any of the aforementioned components either as a single component or any combinations thereof. Typically, the components of the kit are provided in separate containers or within a single container. The container also typically comprises instructions for carrying out the method of the present invention for manufacture of the at least one C-20 terpenoid alcohol. Moreover, the kit may, preferably, comprise further components which are necessary for carrying out the method of the invention such as incubation reagents, cultivation media, washing solutions, solvents, and/or reagents or means required for purification of the at least one C-20 terpenoid alcohol.

The following embodiments are particular preferred embodiments envisaged in accordance with the present invention. All definitions an explanations of the terms made above apply mutatis mutandis.

Embodiment 1: A method for the manufacture of at least one C-20 terpenoid alcohol comprising the steps of:
a) converting geranylgeranyl pyrophosphate into copalyl diphosphate (CPP) or labda-13-en-8-ol diphosphate (LPP); and
b) converting CPP or LPP into at least one C-20 terpenoid alcohol, wherein said conversion is carried out by a polypeptide exhibiting diterpene alcohol synthase activity wherein said diterpene alcohol synthase activity is capable of converting copalyl diphosphate (CPP) into manool, labda-13-en-8-ol diphosphate (LPP) into sclareol and/or LPP into abienol,
wherein said polypeptide comprises and amino acid sequence selected from the group consisting of:
a) an amino acid sequence as shown in any one of SEQ ID NOs: 3 to 10 or SEQ ID NO: 34;
b) an amino acid sequence which is at least 60%, at least 70%, at least 80%, at least 85%, at least 90%, at least 95%, at least 98% or at least 99% identical to the amino acid sequences as shown in any one of SEQ ID NOs: 3 to 10 or SEQ ID NO: 34;
c) an amino acid sequence encoded by a nucleic acid sequence as shown in SEQ ID NO: 1, 2, 16, 17, 18 or 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1, 2, 16, 17, 18 or 35; and
e) an amino acid sequence of a fragment of any one of (a) to (d), said fragment encoding a polypeptide exhibiting a diterpene alcohol synthase activity capable of converting copalyl diphosphate (CPP) into manool, labda-13-en-8-ol diphosphate (LPP) into sclareol and/or LPP into abienol.

Embodiment 2: The method of claim 1, wherein said polypeptide comprises an amino acid sequence of the conserved region as shown in SEQ ID NO: 24.

Embodiment 3: The method of embodiment 1 or 2, wherein said at least one C-20 terpenoid alcohol is a cyclic C-20 terpenoid alcohol.

Embodiment 4: The method of any one of embodiments 1 to 3, wherein said at least one C-20 terpenoid alcohol is manool, sclareol or abienol.

Embodiment 5: The method of any one of embodiments 1 to 4, wherein said polypeptide exhibiting diterpene alcohol synthase activity is capable of converting CPP into manool and LPP into sclareol.

Embodiment 6: The method of embodiment 5, wherein said polypeptide comprises an amino acid sequence selected from the group consisting of:
a) an amino acid sequence as shown in SEQ ID NOs: 4, 6, 7, 9,10 or 34;
b) an amino acid sequence which is at least 60%, at least 70%, at least 80%, at least 85%, at least 90%, at least 95%, at least 98% or at least 99% identical to the amino acid sequences as shown in SEQ ID NOs: 4, 6, 7, 9, 10 or 34;
c) an amino acid sequence encoded by a nucleic acid sequence as shown in SEQ ID NO: 2, 16, 17 or 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 2, 16, 17 or 35; and
e) an amino acid sequence of a fragment of any one of (a) to (d), said fragment encoding a polypeptide exhibiting a diterpene alcohol synthase activity capable of converting CPP into manool and LPP into sclareol.

Embodiment 7: The method of any one of embodiments 1 to 4, wherein said polypeptide exhibiting diterpene alcohol synthase activity is capable of converting LPP into abienol.

Embodiment 8: The method of embodiment 7, wherein said polypeptide comprises an amino acid sequence selected from the group consisting of:
a) an amino acid sequence as shown in SEQ ID NO: 3, 5 or 8;
b) an amino acid sequence which is at least 60%, at least 70%, at least 80%, at least 85%, at least 90%, at least 95%, at least 98% or at least 99% identical to the amino acid sequences as shown in SEQ ID NO: 3, 5 or 8;
c) an amino acid sequence encoded by a nucleic acid sequence as shown in SEQ ID NO: 1 or 18;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1 or 18; and
e) an amino acid sequence of a fragment of any one of (a) to (d), said fragment encoding a polypeptide exhibiting a diterpene alcohol synthase activity capable of converting LPP into abienol.

Embodiment 9: The method of any one of embodiments 1 to 8, wherein said conversion in step a) is carried out by a further polypeptide which exhibits an enzymatic activity of a type II diterpene synthase converting geranylgeranyl pyrophosphate (GGP) into LPP and/or CPP.

Embodiment 10: The method of any one of embodiments 1 to 9, wherein said polypeptide exhibiting diterpene synthase activity is comprised in a fusion polypeptide comprising at least one further polypeptide which exhibits an enzymatic activity of a type II diterpene synthase, preferably, converting geranylgeranyl pyrophosphate (GGP) into LPP and/or CPP, which has maltose binding properties or which is thioredoxin or a thioredoxin fusion protein.

Embodiment 11: The method of embodiment 10, wherein said further polypeptide is selected from the group consisting of: an LPP synthase, preferably, from Coleus forskohlii (CfLPPS) or Nicotiana tabacum (NtLPPS), a CPP synthase, preferably, from Coleus forskohlii (CfCPPS), thioredoxin, and maltose binding protein (MBP).

Embodiment 12: The method of any one of embodiments 1 to 12, wherein said step b) or said steps a) and b) are carried out in a host cell or in a non-human transgenic organism.

Embodiment 13: The method of any one of embodiments 1 to 12, further comprising the step of obtaining said manufactured at least one C-20 terpenoid alcohol.

Embodiment 14: A composition comprising said at least one C-20 terpenoid alcohol, preferably, manool, sclareol and/or abienol obtainable by the method of any one of embodiments 1 to 14.

Embodiment 15: A polypeptide exhibiting diterpene alcohol synthase activity, wherein said diterpene alcohol synthase activity is capable of converting copalyl diphosphate (CPP) into manool, labda-13-en-8-ol diphosphate (LPP) into sclareol and/or LPP into abienol, said polypeptide having an amino acid sequence selected from the group consisting of:
a) an amino acid sequence as shown in any one of SEQ ID NOs: 3 to 10 or SEQ ID NO: 34;
b) an amino acid sequence which is at least 60%, at least 70%, at least 80%, at least 85%, at least 90%, at least 95%, at least 98% or at least 99% identical to the amino acid sequences as shown in any one of SEQ ID NOs: 3 to 10 or SEQ ID NO: 34;
c) an amino acid sequence encoded by a nucleic acid sequence as shown in SEQ ID NO: 1, 2, 16, 17, 18 or 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1, 2, 16, 17, 18 or 35; and
e) an amino acid sequence of a fragment of any one of (a) to (d), said fragment encoding a polypeptide exhibiting a diterpene alcohol synthase activity capable of converting copalyl diphosphate (CPP) into manool, labda-13-en-8-ol diphosphate (LPP) into sclareol and/or LPP into abienol.

Embodiment 16: The polypeptide of embodiment 15, wherein said polypeptide comprises an amino acid sequence of the conserved region as shown in SEQ ID NO: 24.

Embodiment 17: The polypeptide of embodiment 15 or 16, wherein said diterpene alcohol synthase activity is capable of converting CPP into manool and LPP into sclareol.

Embodiment 18: The polypeptide of embodiment 17, wherein said polypeptide comprises an amino acid sequence selected from the group consisting of:
a) an amino acid sequence as shown in SEQ ID NOs: 4, 6, 7, 9, 10 or 34;
b) an amino acid sequence which is at least 60%, at least 70%, at least 80%, at least 85%, at least 90%, at least 95%, at least 98% or at least 99% identical to the amino acid sequences as shown in SEQ ID NOs: 4, 6, 7, 9, 10 or 34;
c) an amino acid sequence encoded by a nucleic acid sequence as shown in SEQ ID NO: 2, 16, 17 or 35;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 2, 16, 17 or 35; and
e) an amino acid sequence of a fragment of any one of (a) to (d), said fragment encoding a polypeptide exhibiting a diterpene alcohol synthase activity capable of converting CPP into manool and LPP into sclareol.

Embodiment 19: The polypeptide of embodiment 15 or 16, wherein said diterpene alcohol synthase activity is capable of converting LPP into abienol.

Embodiment 20: The polypeptide of embodiment 19, wherein said polypeptide comprises an amino acid sequence selected from the group consisting of:
a) an amino acid sequence as shown in SEQ ID NO: 3, 5 or 8;
b) an amino acid sequence which is at least 60%, at least 70%, at least 80%, at least 85%, at least 90%, at least 95%, at least 98% or at least 99% identical to the amino acid sequences as shown in SEQ ID NO: 3, 5 or 8;
c) an amino acid sequence encoded by a nucleic acid sequence as shown in SEQ ID NO: 1 or 18;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 1 or 18; and
e) an amino acid sequence of a fragment of any one of (a) to (d), said fragment encoding a polypeptide exhibiting a diterpene alcohol synthase activity capable of converting LPP into abienol.

Embodiment 21: A fusion polypeptide comprising the polypeptide of any one of embodiments 15 to 20 and at least one further polypeptide (i) which exhibits an enzymatic activity of a type II diterpene synthase, preferably, converting geranylgeranyl pyrophosphate (GGP) into LPP and/or CPP, (ii) which has maltose binding properties or (iii) which is thioredoxin or a thioredoxin fusion protein.

Embodiment 22: The fusion polypeptide of embodiment 21, wherein said further polypeptide is selected from the group consisting of: an LPP synthase, preferably, from Coleus forskohlii (CfLPPS) or Nicotiana tabacum (NtLPPS), a CPP synthase, preferably, from Coleus forskohlii (CfCPPS), thioredoxin, and maltose binding protein (MBP).

Embodiment 23: A polynucleotide encoding the polypeptide of any one of embodiments 15 to 20 or the fusion polypeptide of embodiment 21 or 22 or a reverse complementary or complementary sequence thereof.

Embodiment 24: A vector or gene construct comprising the polynucleotide of embodiment 23.

Embodiment 25: A host cell comprising the vector or gene construct of embodiment 24.

Embodiment 26: The host cell of embodiment 25, wherein said host cell is selected from the group consisting of: a bacterial cell, a yeast cell, a fungal cell, an algal cell or a cyanobacterial cell, a non-human animal cell or a non-human mammalian cell, and a plant cell.

Embodiment 27: A transgenic non-human organism comprising the polynucleotide of embodiment 23, the vector or gene construct of embodiment 24, or the host cell of embodiment 25 or 26.

Embodiment 28: Use of the polypeptide of any one of embodiments 15 to 20 or the fusion polypeptide of embodiment 21 or 22, the polynucleotide of embodiment 23, the vector or gene construct of embodiment 24, the host cell of embodiment 25 or 26, or the non-human transgenic organism of embodiment 27 for the manufacture of at least one C-20 terpenoid alcohol, preferably, abienol, manool, and/or sclareol.

Embodiment 29: The use of embodiment 28, wherein said C-20 terpenoid alcohol is used for producing flavours, agrochemicals, fragrances, pharmaceuticals, cosmetics or chemical building blocks.

Embodiment 30: A kit for the manufacture of at least one C-20 terpenoid alcohol, preferably, abienol, manool, and/or sclareol, comprising the polypeptide of any one of embodiments 15 to 20 or the fusion polypeptide of embodiment 21 or 22, the polynucleotide of embodiment 23, the vector or gene construct of embodiment 24, the host cell of embodiment 25 or 26, or the non-human transgenic organism of embodiment 27.

All references cited throughout this specification are herewith incorporated by reference in their entireties or with respect to the specifically mentioned disclosure content.

### FIGURES

FIG. 1: GC MS analysis of a dichloromethane extract from Cupressus gigantea. A clear manool peak was observed at 19.7 min, corresponding to the Rt of a manool standard.

FIG. 2: GC analysis of strains pBBR-MEV-PcrtE-TrxCfLPPS-mbpCup2v1-PrpIm-CgIsdA and pBBR-MEV-PcrtE-TrxNtLPPS-mbpCup2v1-PrpIm-CgIsdA. A) and b); Analysis revealed a compound eluting at 13.61 min (subsequently identified as abienol).; c, d, e, results from constructs expressing Cup2v2a and Cup2v2b in combination with an LPP Synthase; analysis revealed a new compound eluting at 14.03 min (subsequently identified as sclareol), f) GC analysis of strain pBBR-MEV-PcrtE-TrxCfCPPS-mbpCup2v2a-PrpIm-CgIsdA showed that a compound was produced eluting at 13.29 min (subsequently identified as manool).

FIG. 3: GC MS analysis of strains. a) pBBR-MEV-PcrtE-TrxCfLPPS-mbpCupr2v1-PrpIm-CgIsdA-GC MS analysis confirmed that this compound corresponds to abienol; b) pBBR-MEV-PcrtE-TrxCfLPPS-mbpCupr2v2b-PrpIm-CgIsdA-GC MS analysis confirmed that this compound corresponds to sclareol; c) pBBR-MEV-PcrtE-TrxCfCPPS-mbpCup2v2a-PrpIm-CgIsdA GC MS analysis revealed that this compound corresponds to manool.

FIG. 4: Alignment of product determining region. CfMOS, manoyl oxide synthase from Coleus forskohlii (GenBank accession: KF444508); IrMS, miltiradiene synthase from Isodon rubescens (KX831652); CfMS, miltiradiene synthase from C. forskohlii (KF444509); RoMS1, miltiradiene synthase 1 from Rosemarius officinalis (KF805858); SmMS, miltiradiene synthase from Salvia miltiorrhiza (ABV08817); RoMS1, miltiradiene synthase from Rosemarius officinalis (KF805859); SfMS, miltiradiene synthase from Salvia fruticosa (KP091841); MvELS, 9,13-epoxy-labd-14-ene synthase from Marrubium vulgare (KJ584454); SsSS Salvia sclarea sclareol synthase (JN133922); SsSS-iAS variant of SsSS which is an iso-abienol synthase (Jia et al ACS Catal. 2018, 8, 3133-3137).

FIG. 5: Alignment of the proteins of Cup2v2b (SEQ ID NO: 4), Cup2v2a (SEQ ID NO: 34), Cup2v1 (SEQ ID NO: 3) and TcKSL1, TcKSL2 and TcKSL8 as found at the National Center for Biotechnology Information (NCBI) database under accession numbers KT588484, KT588485 and KT588489, respectively; further the sequence of ScSS as found in SEQ ID NO: 3 of the international patent application WO2009101126.

FIG. 6: Alignment of the proteins of Cup2v2b (SEQ ID NO: 4), Cup2v2a (SEQ ID NO: 34), Cup2v1 (SEQ ID NO: 3) and TcKSL1, TcKSL2 and TcKSL8 as found at the National Center for Biotechnology Information (NCBI) database under accession numbers KT588484, KT588485 and KT588489, respectively.

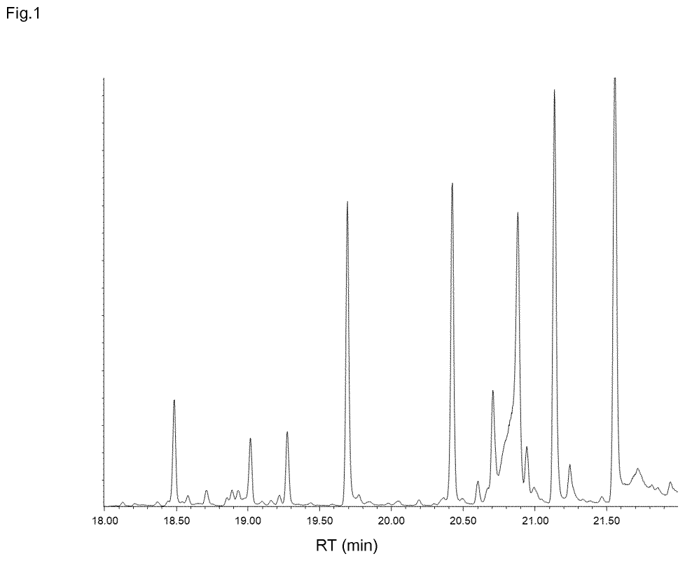

The following sequences are referred to throughout the specification and in the accompanying sequence protocol:
SEQ ID NO: 1: Cup2v1 cDNA sequence
SEQ ID NO: 2: Cup2v2b cDNA sequence
SEQ ID NO: 3: Cup2v1 protein
SEQ ID NO: 4: Cup2v2b protein
SEQ ID NO: 5: truncated Cup2v1 protein
SEQ ID NO: 6: truncated Cup2v2a protein
SEQ ID NO: 7: truncated Cup2v2b protein
SEQ ID NO: 8: MBP-truncated Cup2v1 protein
SEQ ID NO: 9: MBP-truncated Cup2v2a protein
SEQ ID NO: 10: MBP truncated cup2v2b protein
SEQ ID NO: 11: SsSS truncated protein
SEQ ID NO: 12: Trx-CfCPS protein
SEQ ID NO: 13: Trx-CfLPPS protein
SEQ ID NO: 14: Trx-NtLPPS protein
SEQ ID NO: 15: CgIdsA protein
SEQ ID NO: 16: MBP-Cup2v2b DNA
SEQ ID NO: 17: MBP-Cup2v2a DNA
SEQ ID NO: 18: MBP-Cup2v1 DNA
SEQ ID NO: 19: SsSS cDNA
SEQ ID NO: 20: Trx-CfLPPS DNA
SEQ ID NO: 21: Trx-CfCPS DNA
SEQ ID NO: 22: CgidsA cDNA
SEQ ID NO: 23: Trx-NtLPPS DNA
SEQ ID NO: 24: conserved region of Cup2v1, Cup2v2a and Cup2v2b protein
SEQ ID NO: 25: product determining region of CfMOS protein; product determining region of IrMS protein; product determining region of CfMS protein; product determining region of CRoMS1 protein; product determining region of SmMS protein; product determining region of RoMS2 protein; product determining region of SfMS protein; product determining region of MvELS protein
SEQ ID NO: 26: product determining region of SsSS protein
SEQ ID NO: 27: product determining region of SsSS-iAS protein
SEQ ID NO: 28: MBP-Cupr2v2b-2 polypeptide
SEQ ID NO: 29 MBP-Cupr2v2b-3 polypeptide
SEQ ID NO: 30 MBP-Cupr2v2b-4 polypeptide
SEQ ID NO: 31 MBP-Cupr2v2b-2 DNA
SEQ ID NO: 32 MBP-Cupr2v2b-3 DNA
SEQ ID NO: 33 MBP-Cupr2v2b-4 DNA
SEQ ID NO: 34 Cup2v2a protein
SEQ ID NO: 35 Cup2v2a DNA
SEQ ID NO: 36 truncated Cup2v1 DNA
SEQ ID NO: 37 truncated Cup2v2b DNA
SEQ ID NO: 38 truncated Cup2v2a DNA
SEQ ID NO: 39 DNA Cup2v1 double truncated C- and N-terminally
SEQ ID NO: 40 protein Cup2v1 double truncated C- and N-terminally
SEQ ID NO: 41 variant 1 protein
SEQ ID NO: 42 variant 2 protein
SEQ ID NO: 43 variant 3 protein
SEQ ID NO: 44 variant 4 protein
SEQ ID NO: 45 variant 5 protein
SEQ ID NO: 46 variant 6 protein
SEQ ID NO: 47 variant 7 protein
SEQ ID NO: 48 variant 8 protein
SEQ ID NO: 49 variant 9 protein
SEQ ID NO: 50 variant 10 protein
SEQ ID NO: 51 variant 11 protein
SEQ ID NO: 52 variant 12 protein
SEQ ID NO: 53 variant 13 protein
SEQ ID NO: 54 variant 14 protein
SEQ ID NO: 55 Cup motif ENNSFGSMCI
SEQ ID NO: 56 Cup motif EKKSFGSMCI
SEQ ID NO: 57 Cup motif EKNSFGSMCI
SEQ ID NO: 58 Cup motif ENKSFGSMCI

Further, the following polypeptides with the given single amino acid substitutions are also polypeptides according to the invention:
In SEQ ID NO: 4, at position 84, the Lys may be replaced by Asn.
In SEQ ID NO: 6, at position 3, the Asn may be replaced by Lys
In SEQ ID NO: 7, at position 3, the Lys may be replaced by Asn.
In SEQ ID NO: 9, at position 375, the Asn may be replaced by Lys.
In SEQ ID NO: 10, at position 375, the Asn may be replaced by Lys.
In SEQ ID NO: 3, the position 398, is filled with an Ile or a Thr.
In SEQ ID NO: 5, the position 317, is filled with an Ile or a Thr.

### EXAMPLES

The Examples shall merely illustrate the invention. They shall not, whatsoever, be construed as limiting the scope.

### Example 1: Cloning of Cup2v1, Cup2v2a and Cup2v2b

Analysis of Cupressus gigantea Terpenes.

A Cupressus gigantea tree was obtained from EsveId (Boskoop). An extract was prepared from the cortex of the stem by grinding the cortex material to a fine powder under liquid nitrogen, and extracting 100 mg of this powder with 1 ml of dichloromethane. The dichloromethane phase was analysed on a GC MS. A clear manool peak was observed at 19.7 min, corresponding to the Rt of a manool standard.

RNA Extraction was Performed and Sequencing from cDNA of Cupressus Tissue

About 15 mL extraction buffer (2% hexadecyl-trimethylammonium bromide, 2% polyvinylpyrrolidinone K 30, 100 mM Tris-HCl (pH 8.0), 25 mM EDTA, 2.0 M NaCl, 0.5 g/L spermidine and 2% β-mercaptoethanol) was warmed to 65° C., after which 3 g ground cortex tissue was added and mixed. The mixture was extracted two times with an equal volume of chloroform: isoamylalcohol (1:24), and one-fourth volume of 10 M LiCl was added to the supernatant and mixed. The RNA was precipitated overnight at 4° C. and harvested by centrifugation at 10 000 g for 20 min. The pellet was dissolved in 500 μL of SSTE [1.0 M NaCl, 0.5% SDS, 10 mM Tris-HC1 (pH 8.0), 1 mM EDTA (pH 8.0)] and extracted once with an equal volume of chloroform: isoamylalcohol. Two volumes of ethanol were added to the supernatant, incubated for at least 2 h at −20° C., centrifuged at 13 000 g and the supernatant removed. The pellet was air-dried and resuspended in water. Total RNA (60 μg) was shipped to Vertis Biotechnology AG (Freising, Germany). PolyA+ RNA was isolated, random primed cDNA synthesized using a randomized N6 adapter primer and M-MLV H-reverse transcriptase. cDNA was sheared and fractionated, and fragments of a size of 500 bp were used for further analysis. The cDNAs carry attached to their 5′ and 3′ ends the adaptor sequences A and B as specified by Illumina. The material was subsequently analysed on a Illumina MiSeq Sequencing device. In total, 19,608,859 sequences were read by the MiSeq. Trimmomatic-0.32 was used to trim sequences from Illumina sequencing adapters, Seqprep was used to overlap paired end sequences, and bowtie2 (version 2.2.1) was used to remove phiX contamination (phiX DNA is used as a spike-in control, usually present in <1%). Paired end reads and single reads were used in a Trinity assembly (trinityrnaseq-2.0.2). A total number of 88667 contigs were assembled by Trinity.

In order to identify sesquiterpene synthases, the C. gigantea contigs were used to create a database of cDNA sequences. In this database, the TBLASTN program was deployed to identify cDNA sequences that encode proteins that show identity with protein sequences of sesquiterpene synthases, including kaurene synthase from Arabidopsis thaliana (Q9SAK2), sclareol synthase from Salvia sclarea (AET21246.1), abienol synthase from Abies balsamifera (H8ZM73.1), 13-labden-8,15-diol pyrophosphate synthase from Salvia sclarea (AET21248.1). In total 184 contigs in the C. giganteaa cDNA database were identified which have significant homology to sesquiterpene synthases. The contigs were grouped into 68 groups according to their overlap in sequence. These 68 contigs were further characterized by analyzing them using the BLASTX program to align them to protein sequences present in the UniProt database (downloaded Aug. 28, 2015), and the inventors identified by hand, 12 of them as putative diterpene synthase sequences, according to their homology to terpene synthases sequences present in UniProt and their features.

### Identification of Cup2v1, Cup2v2a and Cup2v2b

Three of cDNA sequences were selected by the inventors as the most promising candidate genes based on the skilful analysis of their features. The cDNA sequences shown in SEQ ID Nos. 1 and 2 were identified as Cup2v1 and Cup2v2b, respectively. Cup2v1 protein is shown in SEQ ID NO: 3 and Cup2v2b protein is shown in SEQ ID NO: 4. Cup2v1 and Cup2v2b proteins are 93.8% identical to each other on amino acid level.

The third cDNA sequence was similar to Cup2v2b and was designated Cup2v2a. The inventors generated artificially shortened version of the sequence, thereby removing the plastid targeting signal and changing the N-terminus. These truncated amino acid sequences (named trcup2v1, trcup2v2a and trcup2v2b) are given in SEQ ID NO: 5 to 7, respectively. Full length Cup2v2a protein is shown in SEQ ID NO: 34, the cDNA sequence is depicted in SEQ ID NO: 35.

Of the known Salvia sclareol synthase (SsSS) a truncated version was created as control (trSsSS).

BLAST in NCBI nr protein database reveals that the closest homologue of these proteins is a diterpene synthase with unknown product specificity from Taiwania cryptomerioides (AOG18231.1) with an amino acid 67.6% identity. BLAST in uniprot database of characterized proteins reveals ent-kaurene synthase from Vitex agnuscastus with an amino acid 39.1% identity.

#TOOL: needle

#GAPMETHOD: NOGAPS

#GAPOPEN: 10, GAPEXTEND: 0.5, MATRIX: EBLOSUM62

| | Cup2v1 | Cup2v2b | trcup2v1 | trcup2v2a | trCup2v2b | trSsSS | ScSS |
|---|---|---|---|---|---|---|---|
| Cup2v1 | 100.0% | 93.8% | 99.8% | 92.7% | 92.7% | 31.3% | 31.0% |
| Cup2v2b | — | 100.0% | 92.7% | 99.1% | 99.8% | 30.8% | 29.7% |
| trcup2v1 | — | — | 100.0% | 92.9% | 92.9% | 31.3% | 31.2% |
| trcup2v2a | — | — | — | 100.0% | 99.3% | 31.2% | 30.5% |
| trCup2v2b | — | — | — | — | 100.0% | 30.6% | 29.4% |
| trSsSS | — | — | — | — | — | 100.0% | 100.0% |
| ScSS | — | — | — | — | — | — | 100.0% |

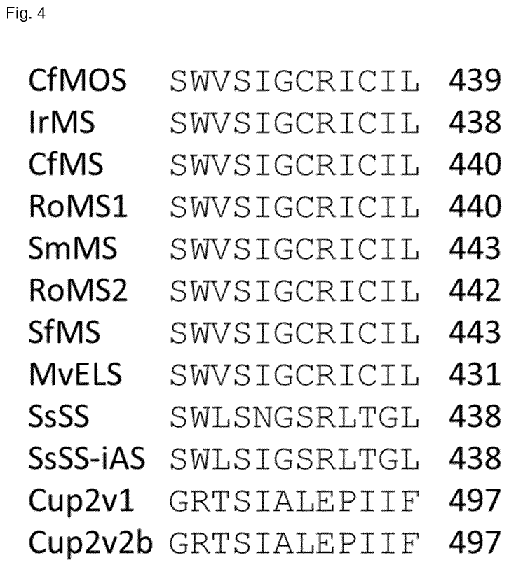

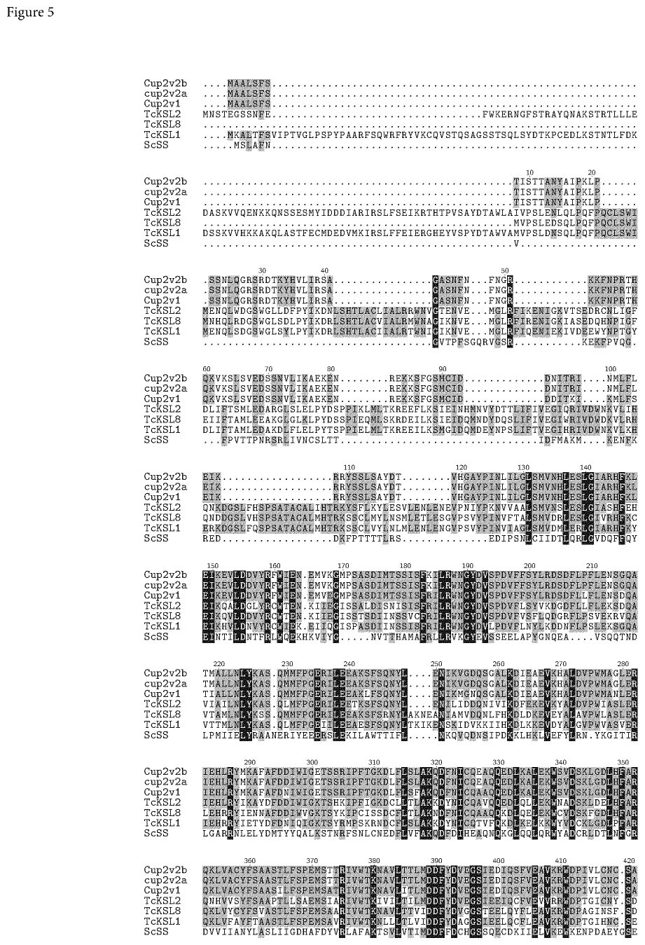

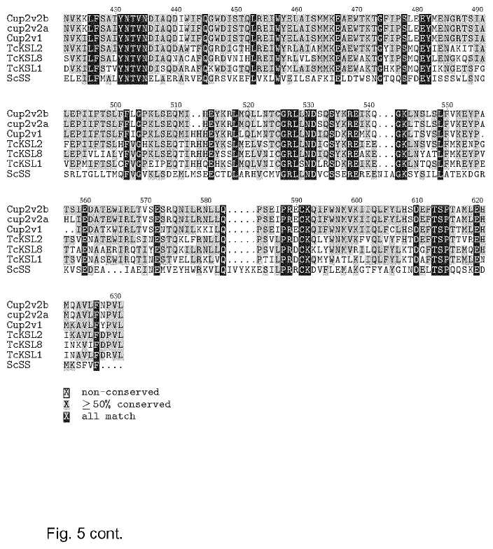

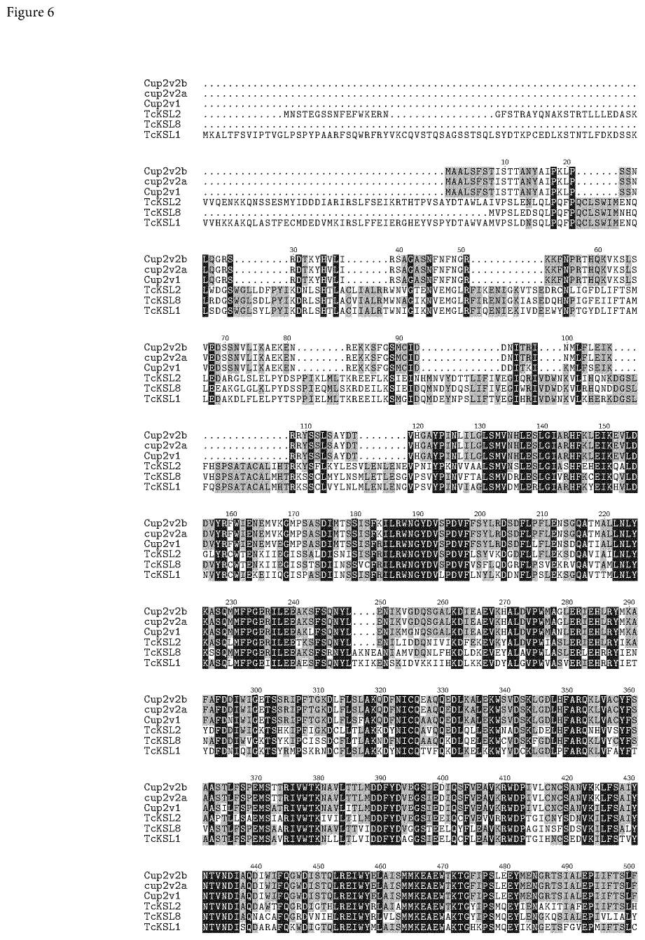

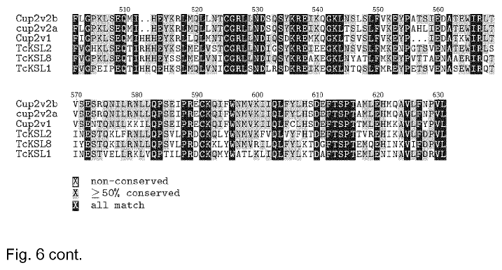

Cup2v1, Cup2v2a and Cup2v2b proteins have been identified by the inventors to be candidates for step 2 diterpene alcohol synthases for generating abienol, manool and/or sclareol. An essentially conserved region was identified by the inventors between Cup2v1, Cup2v2a and Cup2v2b (see alignment FIG. 4). This region in the synthases is located at a location corresponding to the product determining region of other synthases but different from the product determining region of said other synthases including the product determining region in the known Salvia sclareol synthase. Although Cup2v1, Cup2v2a and Cup2v2b have different product specificity (see below), the region typically responsible for determining product specificity in other diterpene synthases known is very different yet conserved between said Cup proteins.

### Example 2: Construction of Plasmids for Expression of Step 1 and Step 2 Genes in Rhodobacter

For expression in Rhodobacter, fusion proteins were designed for the truncated versions of Cup2v1, Cup2v2a, Cup2v2b with the maltose binding protein (named mbpCup2v1, mbpCup2v2a and mbpCup2v2b, see SEQ ID NO: 8 to 10, respectively), and for a number of step 1 genes CfLPPS, CfCPPS, and NtLPPS fusion proteins with thioredoxin Trx (see SEQ ID Nos: 12 to 14). For comparison, also a construct was prepared expressing CfLPPS in combination with a truncated version of Salvia sclarea Sclareol synthase (SsSS). This truncated version corresponds to the SsSS as it was published in Schalk J. Am. Chem. Soc. 2012, 134, 18900-18903.

A construct was made where the mevalonate operon from Paracoccus zeaxanthinifaciens was expressed with its native promoter as described in EP 2 336 310 A1, together with CgIdsA, expressed from an Lppa promoter as described in WO 2018/160066 AI, and an operon comprising the crtE promoter, followed by a trx-step 1 gene, a ribosome binding site and an mbp-step2 gene.

The following set of constructs was prepared
a. pBBR-MEV-PcrtE-TrxNtLPPS-mbpCup2v1-PrpIm-CgIsdA
b. pBBR-MEV-PcrtE-TrxCfLPPS-mbpCup2v1-PrpIm-CgIsdA
C. pBBR-MEV-PcrtE-TrxNtLPPS-mbpCup2v2a-PrpIm-CgIsdA
d. pBBR-MEV-PcrtE-TrxCfLPPS-mbpCup2v2a-PrpIm-CgIsdA
e. pBBR-MEV-PcrtE-TrxCfLPPS-mbpCup2v2b-PrpIm-CgIsdA
f. pBBR-MEV-PcrtE-TrxCfCPPS-mbpCup2v2a-PrpIm-CgIsdA
g. pBBR-MEV-PcrtE-TrxCfLPPS-SsSS-PrpIm-CgIsdA

These constructs were introduced in E. coli S17-1, and resulting strains were used for conjugation to Rhodobacter sphaeroides Rs265-9c by using standard procedures. Resulting strains were named after their plasmids.

### Example 3: Small Scale Recombinant Manufacture of C-20 Terpenoid Alcohols

Each strain was used for a small-scale production test, basically as has been described in US2020/0010822A1. To this end, seed cultures were performed in 100 ml shake flasks without baffles with 20 ml RS102 medium with 100 mg/L neomycin and a loop of glycerol stock. Seed culture flasks were grown for 72 hours at 30° C. in a shaking incubator with an orbit of 50 mm at 110 rpm.

At the end of the 72 hours, the OD600 of the culture was assessed in order to calculate the exact volume of culture to be transferred to the larger flasks.

Shake flask experiments were performed in 300 ml shake flasks with 2 bottom baffles. Twenty ml of RS102 medium and neomycin to a final concentration of 100 mg/L were added to the flask together with 2 ml of sterile n-dodecane. The volume of the inoculum was adjusted to obtain a final OD600 value of 0.05 in 20 ml medium.

The flasks were kept for 72 hours at 30° C. in a shaking incubator with an orbit of 50 mm at 110 rpm. Subsequently, cultures were collected in pre-weighted 50 ml PP tubes which were then centrifuged at 4500×g for 20 minutes. The n-dodecane layer was transferred to a microcentrifuge tube for later GC analysis.

Ten microliters of ethyl laureate were weighed in a 10-ml glass vial to which 800 μl of the isolated dodecane solution were added and weighed. Subsequently, 8 ml of acetone were added to the vial to dilute the dodecane concentration for a more accurate GC analysis. Approximately, 1.5 ml of the terpene-containing dodecane in acetone solution were transferred to a chromatography vial. Each sample was analyzed by gas chromatography, as described in US2020/0010822A1. For compound identification, about 2 μL was analyzed by GC/MS using a gas chromatograph as described in detail by Cankar et al. (2015). Products were identified by the comparison of retention times and mass spectra to those of standards of sclareol, manool and abienol (Sigma-Aldrich).

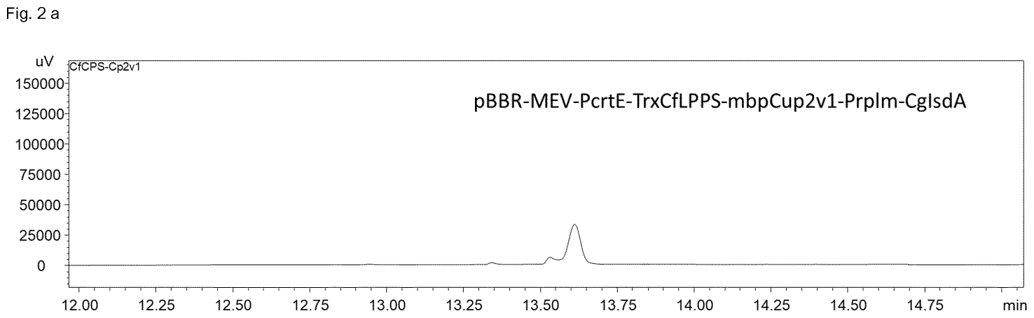

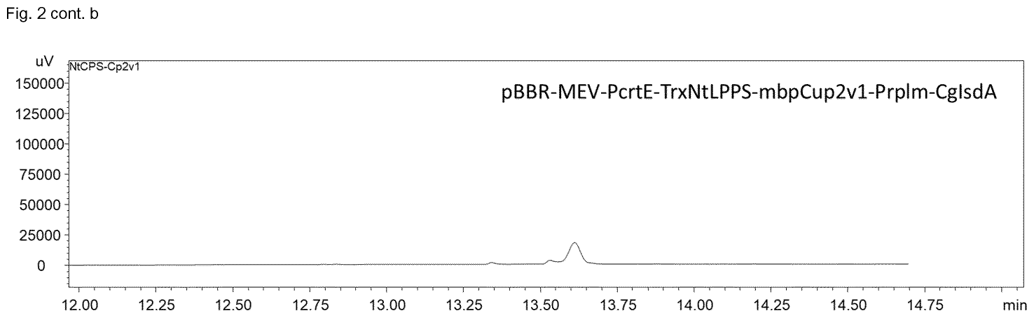

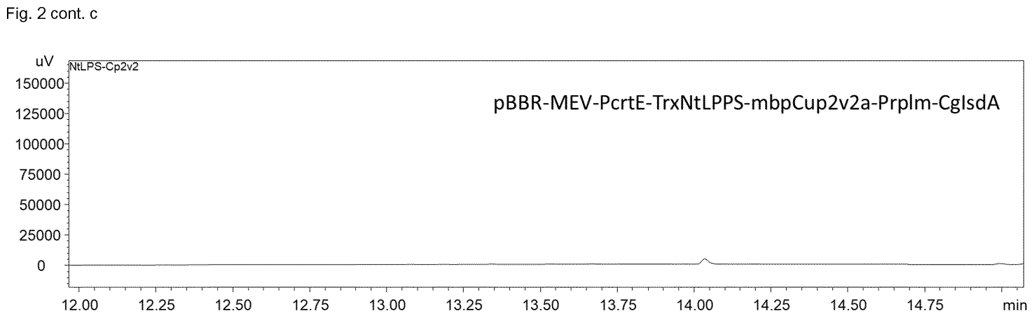

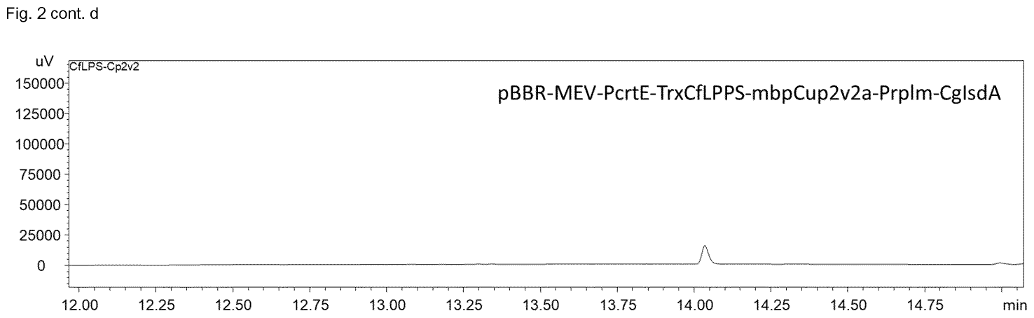

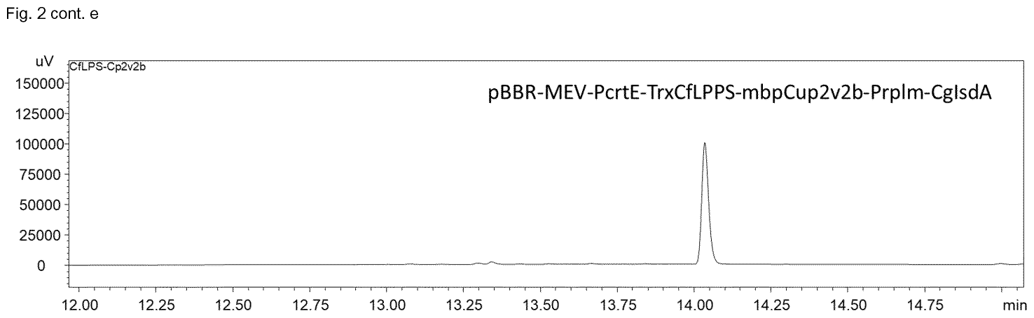

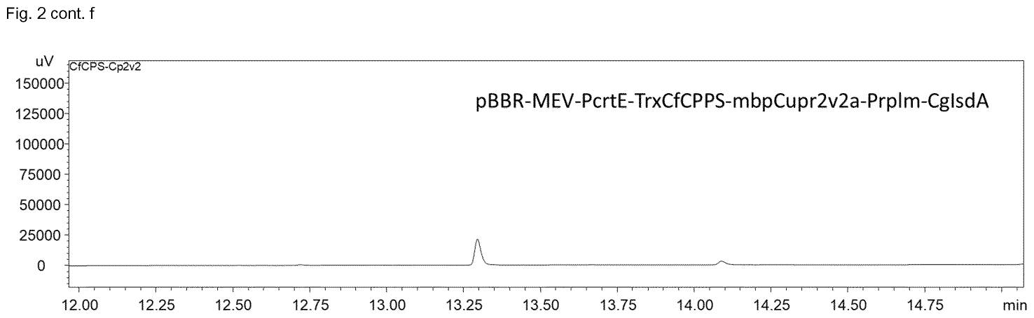

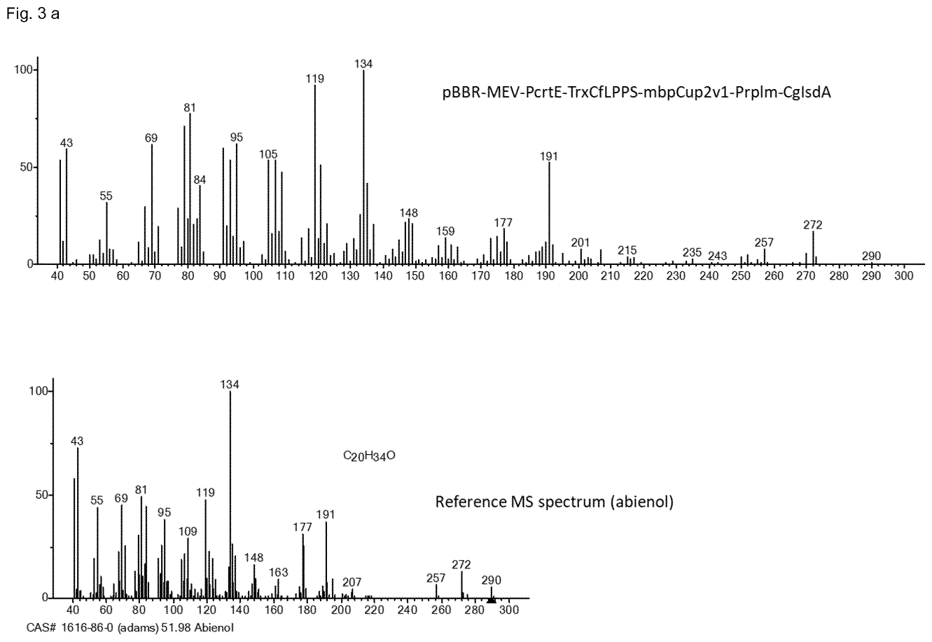

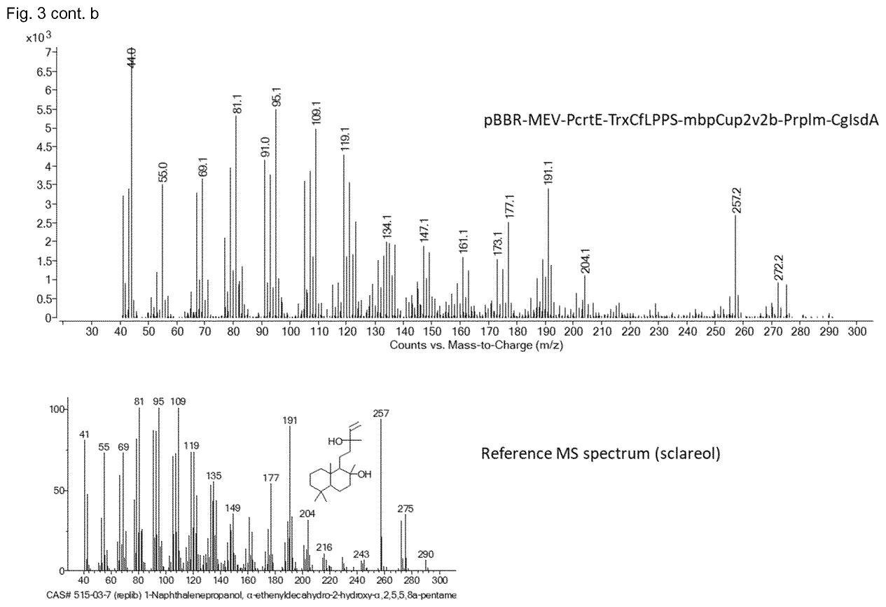

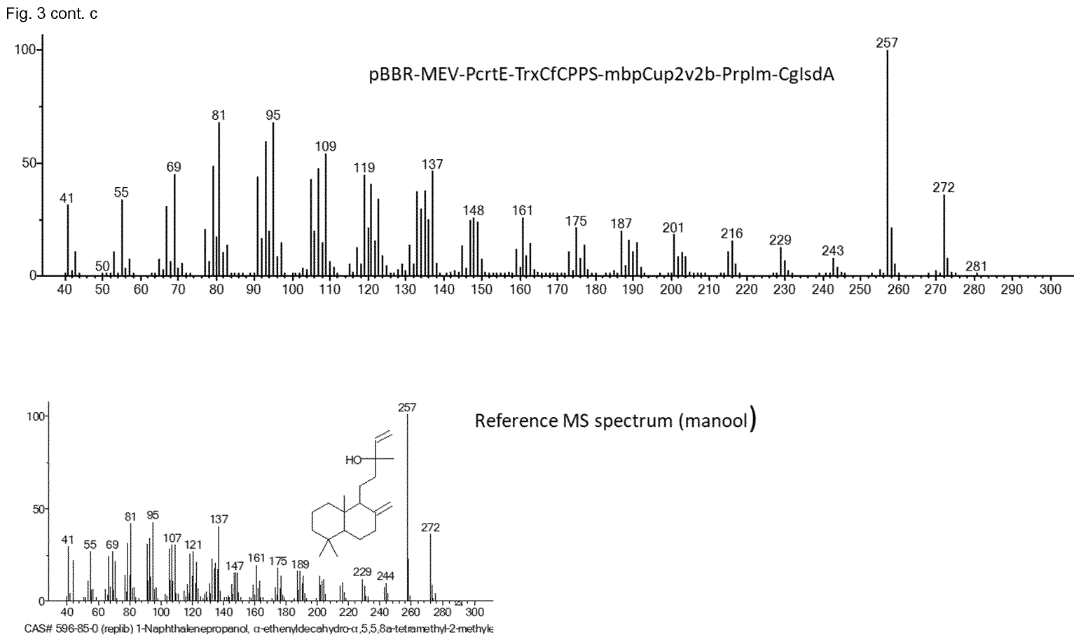

GC analysis of strains pBBR-MEV-PcrtE-TrxNtLPPS-mbpCup2v1-PrpIm-CgIsdA and pBBR-MEV-PcrtE-TrxCfLPPS-mbpCup2v1-PrpIm-CgIsdA revealed a compound eluting at 13.61 min (FIG. 2a, b). GC MS analysis confirmed that this compound corresponds to abienol (FIG. 3a). For abienol, the following titers (g/kg n-dodecane) have been found with the constructs: 1.9 for pBBR-MEV-PcrtE-TrxNtLPPS-mbpCup2v1-PrpIm-CgIsdA and 3.5 for pBBR-MEV-PcrtE-TrxCfLPPS-mbpCup2v1-PrpIm-CgIsdA.

GC analysis of strain pBBR-MEV-PcrtE-TrxCfCPPS-mbpCup2v2a-PrpIm-CgIsdA (FIG. 2f) showed that a compound was produced eluting at 13.29 min. GC MS analysis revealed that this compound corresponds to manool (FIG. 3c). For manool, the following titers (g/kg n-dodecane) have been found with the construct: 1.5 for pBBR-MEV-PcrtE-TrxCfCPPS-mbpCup2v2a-PrpIm-CgIsdA

GC analysis of strains pBBR-MEV-PcrtE-TrxNtLPPS-mbpCup2v2a-PrpIm-CgIsdA, pBBR-MEV-PcrtE-TrxCfLPPS-mbpCup2v2a-PrpIm-CgIsdA, pBBR-MEV-PcrtE-TrxCfLPPS-SsSS-Prplm-CglsdA and pBBR-MEV-PcrtE-TrxCfLPPS-mbpCup2v2b-PrpIm-CgIsdA revealed a new compound eluting at 14.03 min (FIG. 2c, d, e). GC MS analysis confirmed that this compound corresponds to sclareol (FIG. 3b). A quantitative analysis for sclareol with different constructs is shown in the table, below:

TABLE 1
Sclareol relative amounts

| Strain | Relative to SsSS | product |
|---|---|---|
| pBBR-MEV-PortE-TrxNtLPPS- | 31% | sclareol |
| mbpCup2v2a-Prplm-CglsdA | | |
| pBBR-MEV-PcrtE-TrxCfLPPS- | 54% | sclareol |
| mbpCup2v2a-Prplm-CglsdA | | |
| pBBR-MEV-PcrtE-TrxCfLPPS- | 133% | sclareol |
| mbpCup2v2b-Prplm-CglsdA | | |
| pBBR-MEV-PcrtE-TrxCfLPPS-SsSS- | 100% | sclareol |
| Prplm-CglsdA (control) | | |

where the titre in g per kg n-dodecane was normalised of the one achieved with the control.

Further sequence variants of Cup2v2b with additional sequences at the N terminus compared to SEQ ID NO: 7 were also tested as fusion proteins with an N-terminal MBP (SEQ ID NO: 28 to 30), in a similar set-up. All three showed similar levels of sclareol production as shown for pBBR-MEV-PcrtE-TrxCfLPPS-mbpCup2v2b-PrpIm-CgIsdA in the Table 1, line 4, above.

1.-16. (canceled)

17. A method for the manufacture of at least one C-20 terpenoid alcohol comprising the steps of:
a) converting geranylgeranyl pyrophosphate into copalyl diphosphate (CPP) or labda-13-en-8-ol diphosphate (LPP); and
b) converting CPP or LPP into at least one C-20 terpenoid alcohol, wherein said conversion is carried out by a polypeptide exhibiting diterpene alcohol synthase activity, wherein said diterpene alcohol synthase activity is capable of converting CPP into manool, LPP into sclareol and/or LPP into abienol,
wherein said polypeptide comprises an amino acid sequence selected from the group consisting of:
i) an amino acid sequence as shown in SEQ ID NO: 3, 4, 5, 6, 7, 8, 9, 10, or 34;
ii) an amino acid sequence which is at least 60% identical to the amino acid sequence of SEQ ID NO: 3, 4, 5, 6, 7, 8, 9, 10, or 34;
iii) an amino acid sequence encoded by a nucleic acid sequence as shown in SEQ ID NO: 1, 2, 16, 17, 18, or 35;
iv) an amino acid sequence encoded by a nucleic acid sequence which is at least 60% identical to the nucleic acid sequence as shown in SEQ ID NO: 1, 2, 16, 17, 18, or 35; and
v) a fragment of the amino acid sequence of (i), (ii), (iii), or (iv), said fragment encoding a polypeptide exhibiting a diterpene alcohol synthase activity capable of converting CPP into manool, LPP into sclareol and/or LPP into abienol.

18. The method of claim 17, wherein said polypeptide exhibiting diterpene alcohol synthase activity is capable of converting CPP into manool and LPP into sclareol and wherein said polypeptide comprises an amino acid sequence selected from the group consisting of:
a) an amino acid sequence as shown in SEQ ID NO: 4, 6, 7, 9, 10, or 34;
b) an amino acid sequence which is at least 60% identical to the amino acid sequence of SEQ ID NO: 4, 6, 7, 9, 10, or 34;
c) an amino acid sequence encoded by a nucleic acid sequence as shown in SEQ ID NO: 2, 16, 17, or 35;
d) an amino acid sequence encoded by a nucleic acid sequence which is at least 60% identical to the nucleic acid sequence of SEQ ID NO: 2, 16, 17, or 35; and
e) a fragment of the amino acid sequence of (a), (b), (c), or (d), said fragment encoding a polypeptide exhibiting a diterpene alcohol synthase activity capable of converting CPP into manool and LPP into sclareol.

19. The method of claim 17, wherein said polypeptide exhibiting diterpene alcohol synthase activity is capable of converting LPP into abienol; and wherein said polypeptide comprises an amino acid sequence selected from the group consisting of:
a) an amino acid sequence as shown in SEQ ID NO: 3, 5, or 8;
b) an amino acid sequence which is at least 60% identical to the amino acid sequence of SEQ ID NO: 3, 5, or 8;
c) an amino acid sequence encoded by a nucleic acid sequence as shown in SEQ ID NO: 1 or 18;
d) an amino acid sequence encoded by a nucleic acid sequence which is at least 60% identical to the nucleic acid sequence as shown in SEQ ID NO: 1 or 18; and
e) a fragment of the amino acid sequence of (a), (b), (c), or (d), said fragment encoding a polypeptide exhibiting a diterpene alcohol synthase activity capable of converting LPP into abienol.

20. The method of claim 17, wherein said conversion in step a) is carried out by a further polypeptide which exhibits an enzymatic activity of a type II diterpene synthase converting geranylgeranyl pyrophosphate (GGP) into LPP and/or CPP.

21. The method of claim 17, wherein said step b) or said steps a) and b) are carried out in a host cell or in a non-human transgenic organism.

22. A composition comprising a host cell or a non-human transgenic organism, and manool, sclareol and/or abienol obtainable by the method of claim 17, wherein the host cell or the non-human transgenic organism comprises a polypeptide comprising the amino acid sequence of i), ii), iii), iv), or the fragment of v).

23. A polypeptide exhibiting diterpene alcohol synthase activity, wherein said diterpene alcohol synthase activity is capable of converting copalyl diphosphate (CPP) into manool, labda-13-en-8-ol diphosphate (LPP) into sclareol and/or LPP into abienol, said polypeptide having an amino acid sequence selected from the group consisting of:
a) an amino acid sequence as shown in SEQ ID NO: 3, 4, 5, 6, 7, 8, 9, 10, or 34;
b) an amino acid sequence which is at least 60% identical to the amino acid sequences as shown in SEQ ID NO: 3, 4, 5, 6, 7, 8, 9, 10, or 34;
c) an amino acid sequence encoded by a nucleic acid sequence as shown in SEQ ID NO: 1, 2, 16, 17, 18, or 35;
d) an amino acid sequence encoded by a nucleic acid sequence which is at least 60% identical to the nucleic acid sequence as shown in SEQ ID NO: 1, 2, 16, 17, 18, or 35; and
e) a fragment of the amino acid sequence of (a), (b), (c), or (d), said fragment encoding a polypeptide exhibiting a diterpene alcohol synthase activity capable of converting CPP into manool, LPP into sclareol and/or LPP into abienol.

24. The polypeptide of claim 23, wherein said diterpene alcohol synthase activity is capable of converting CPP into manool and LPP into sclareol; and wherein said polypeptide comprises an amino acid sequence selected from the group consisting of:
a) an amino acid sequence as shown in SEQ ID NO: 4, 6, 7, 9, 10, or 34;
b) an amino acid sequence which is at least 60%, identical to the amino acid sequence of SEQ ID NO: 4, 6, 7, 9, 10, or 34;
c) an amino acid sequence encoded by a nucleic acid sequence as shown in SEQ ID NO: 2, 16, 17, or 35;
d) an amino acid sequence encoded by a nucleic acid sequence which is at least 60% identical to the nucleic acid sequence as shown in SEQ ID NO: 2, 16, 17, or 35; and
e) a fragment of the amino acid sequence of (a), (b), (c), or (d), said fragment encoding a polypeptide exhibiting a diterpene alcohol synthase activity capable of converting CPP into manool and LPP into sclareol.

25. The polypeptide of claim 23, wherein said diterpene alcohol synthase activity is capable of converting LPP into abienol and, wherein said polypeptide comprises an amino acid sequence selected from the group consisting of:
a) an amino acid sequence as shown in SEQ ID NO: 3, 5 or 8;
b) an amino acid sequence which is at least 60% identical to the amino acid sequence of SEQ ID NO: 3, 5, or 8;
c) an amino acid sequence encoded by a nucleic acid sequence as shown in SEQ ID NO: 1 or 18;
d) an amino acid sequence encoded by a nucleic acid sequence which is at least 60%, identical to the nucleic acid sequence of SEQ ID NO: 1 or 18; and
e) a fragment of the amino acid sequence of (a), (b), (c), or (d), said fragment encoding a polypeptide exhibiting a diterpene alcohol synthase activity capable of converting LPP into abienol.

26. A fusion polypeptide comprising the polypeptide of claim 23 and at least one further polypeptide, wherein the at least one further polypeptide:
(i) exhibits an enzymatic activity of a type II diterpene synthase;
(ii) has maltose binding properties; or
(iii) is thioredoxin or a thioredoxin fusion protein.

27. A polynucleotide encoding the polypeptide of claim 23 or a reverse complementary or complementary sequence thereof.

28. A vector or gene construct comprising the polynucleotide of claim 27.

29. A host cell comprising the vector or gene construct of claim 28.

30. A transgenic non-human organism comprising the polynucleotide of claim 27.

31. Use of the polypeptide of claim 23 for the manufacture of at least one C-20 terpenoid alcohol.

32. A method for preparing a variant polypeptide having a diterpene alcohol synthase activity comprising the steps of:
a) selecting a nucleic acid according to claim 27;
b) modifying the selected nucleic acid to obtain at least one mutant nucleic acid;
c) transforming a host cell or a unicellular organism with the mutant nucleic acid sequence to express a polypeptide encoded by the mutant nucleic acid sequence;
d) screening the polypeptide for at least one modified property as well as diterpene alcohol synthase activity; and
e) optionally, if the polypeptide has no desired variant diterpene alcohol synthase activity, repeating steps (a), (b), and (c) until a polypeptide with variant diterpene alcohol synthase activity is obtained; and
f) optionally, if a polypeptide having variant diterpene alcohol synthase activity was identified in step (d), isolating the corresponding mutant nucleic acid obtained in step (c).
